(19) United States
(12) Patent Application Publication
(10) Pub. No.: US 2025/0281538 A1
(43) Pub. Date: Sep. 11, 2025

(54) COMPOSITIONS TARGETING GPC2 AND GPC3 AND THEIR USE FOR TREATING SOLID TUMORS

(71) Applicants: The U.S.A., as represented by the Secretary, Department of Health and Human Services, Bethesda, MD (US); Eureka Therapeutics, Inc., Emeryville, CA (US)

(72) Inventors: Mitchell Ho, Urbana, MD (US); Nan Li, Laurel, MD (US); Dan Li, Rockville, MD (US); Cheng Liu, Orinda, CA (US); Hongbing Zhang, Moraga, CA (US); Zhiyuan Yang, Richmond, CA (US)

(73) Assignees: The U.S.A., as represented by the Secretary, Department of Health and Human Services, Bethesda, MD (US); Eureka Therapeutics, Inc., Emeryville, CA (US)

(21) Appl. No.: 18/861,737

(22) PCT Filed: May 2, 2023
(86) PCT No.: PCT/US2023/066487

§ 371 Date: Oct. 30, 2024

Related U.S. Application Data
(60) Provisional application No. 63/337,296, filed on May 2, 2022.

(51) Int. Cl. A61K 35/17; A61K 40/11; A61K 40/31; A61K 40/32; A61K 40/42; A61P 35/00; C07K 14/725; C07K 16/30; C12N 5/0783

(52) U.S. Cl. CPC A61K 35/17; A61K 40/11; A61K 40/31; A61K 40/32; A61K 40/4261; A61P 35/00; C07K 14/7051; C07K 16/303; C12N 5/0636; A61K 2239/13; A61K 2239/21; A61K 2239/47; C07K 2317/35; C07K 2317/565; C07K 2317/569; C07K 2317/622; C07K 2319/03

(57) ABSTRACT

Recombinant antibody T cell receptors (AbTCRs) and chimeric signaling receptors (CSRs) engineered to include antigen-binding domains specific for tumor antigen glypican-2 (GPC2) or glypican-3 (GPC3) are described. Immune cells expressing the AbTCRs and CSRs effectively treated GPC2-positive or GPC3-positive tumors in multiple different xenograft models, and were significantly more potent than similarly targeted chimeric antigen receptor (CAR) T cells.

### CROSS REFERENCE TO RELATED APPLICATIONS

This application claims the benefit of U.S. Provisional Application No. 63/337,296, filed May 2, 2022, which is herein incorporated by reference in its entirety.

### STATEMENT OF GOVERNMENT SUPPORT

This invention was made with government support under Z01 BC010891 awarded by the National Institutes of Health, National Cancer Institute. The government has certain rights in the invention.

### FIELD

This disclosure concerns nucleic acid molecules encoding antibody T cell receptors (AbTCRs) and/or chimeric signaling receptors (CSRs), immune cells expressing the AbTCRs and CSRs, and methods of their use for treating solid tumors that express glypican-2 (GPC2) or glypican-3 (GPC3).

### PARTIES TO JOINT RESEARCH AGREEMENT

The subject matter disclosed and claimed herein was made as a result of activities undertaken within the scope of a joint research agreement between the National Cancer Institute, an agency of the United States Government, and Eureka Therapeutics, Inc.

### INCORPORATION OF ELECTRONIC SEQUENCE LISTING

The electronic sequence listing, submitted herewith as an XML file named 4239-107435-02.xml (82,874 bytes), created on Apr. 19, 2023, is herein incorporated by reference in its entirety.

### BACKGROUND

Chimeric antigen receptor (CAR) T cell therapy has emerged as a new class of cancer therapeutics that is being actively developed and tested worldwide. With initial positive results in hematological cancers using CD19-targeted CAR T cells, various CAR strategies have been engineered and tested with the goal of treating solid tumors, but they have had limited success.

Neuroblastoma is the most common extracranial solid tumors in children. Derived from neuroendocrine tissue of the sympathetic nervous system, it accounts for 8-10% of childhood cancers in the USA (Maris and Hogarty, Lancet 369:2106-2120, 2007). Neuroblastoma is a complex and heterogeneous disease, with nearly 50% of patients having a high-risk phenotype characterized by widespread dissemination of the cancer and poor long-term survival even if intensive multimodal treatments are used (Yu et al., New Engl J Med 363:1324-1334, 2010). Glypican-2 (GPC2) is uniquely expressed in the nervous system (Stipp et al., J Cell Biol 124:149-160, 1994), and is highly expressed in neuroblastoma and other pediatric cancers (Orentas et al., Front Oncol 2:194, 2012; Li et al., Proc Natl Acad Sci USA 114 (32): E6623-E6631, 2017). Approximately 45% of patients receiving a standard therapy have a relapse and ultimately succumb to metastatic disease (Matthay et al., New Engl J Med 341:1165-1173, 1999).

Hepatocellular carcinoma (HCC) is a highly aggressive type of tumor with a poor prognosis. Glypican-3 (GPC3), a cell surface oncofetal protein, is highly upregulated in HCC, but is either not expressed or expressed at low levels in normal tissues, making GPC3 a viable target for cancer immunotherapy (Ho and Kim, Eur J Cancer 47 (3): 333-338, 2011). GPC3-targeted CAR T cells have demonstrated limited efficacy in a Phase 1 clinical trial, likely due to the combination of a lack of adequate tumor penetration and an inability to induce a durable, potent immune response within the confines of the tumor.

Thus, a need exists to develop effective immunotherapies for the treatment of GPC2- and GPC3-expressing solid tumors.

### SUMMARY

Described herein are recombinant antibody T cell receptors (AbTCRs) and chimeric signaling receptors (CSRs) engineered to include antigen-binding domains specific for tumor antigen glypican-2 (GPC2) or glypican-3 (GPC3). In multiple different xenograft models, immune cells co-expressing the disclosed AbTCRs and CSRs effectively treated GPC2-positive or GPC3-positive tumors, and were significantly more potent than similarly targeted CAR T cells.

Provided herein are nucleic acid molecules or sets of nucleic acid molecules encoding an AbTCR and/or a CSR targeted to either GPC2 or GPC3. The disclosed nucleic acid molecules or sets of nucleic acid molecules include a first module that includes a nucleic acid sequence encoding a first antigen-binding polypeptide, and a nucleic acid sequence encoding a co-stimulatory immune cell signaling domain; a second module that includes a nucleic acid sequence encoding a second antigen-binding polypeptide, and a nucleic acid sequence encoding a first TCR chain or a transmembrane domain-containing fragment thereof; and a third module that includes a nucleic acid sequence encoding a third antigen-binding polypeptide, and a nucleic acid sequence encoding a second TCR chain or a transmembrane domain-containing fragment thereof. The first, second and third antigen-binding polypeptides specifically bind GPC2, or the first, second and third antigen-binding polypeptides specifically bind GPC3.

In some aspects, a single nucleic acid molecule includes the first module, the second module and the third module, thus a single nucleic acid molecule encodes both the AbTCR and CSR. In other aspects, the set of nucleic acid molecules includes a first nucleic acid molecule that includes the first module and a second nucleic acid molecule that includes the second module and the third module. In these aspects, the first nucleic acid molecule encodes the CSR and the second nucleic acid molecule encodes both chains of the AbTCR. In yet other aspects, the set of nucleic acid molecules includes a first nucleic acid molecule that includes the first module, a second nucleic acid molecule that includes the second module, and a third nucleic acid molecule that includes the third module. In these aspects, the first nucleic acid molecule encodes the CSR, the second nucleic acid molecule encodes one chain of the AbTCR and the third nucleic acid molecule encodes the second chain of the AbTCR.

In some aspects, the first module further includes a nucleic acid sequence encoding a protein tag or linker (such as a Myc tag).

In some aspects, the co-stimulatory immune cell signaling domain includes all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds to CD83.

In some aspects in which a single nucleic acid molecule includes the first, second and third modules, each module is separated by a nucleic acid sequence encoding a self-cleaving peptide, such as a 2A peptide.

Vectors or sets of vectors that include a disclosed nucleic acid molecule or sets of nucleic acid molecules are further provided. In addition, isolated cells that include a nucleic acid molecule, set of nucleic acid molecules, vector or set of vectors disclosed herein are also provided.

In some aspects in which the AbTCR and CSR are targeted to GPC2, the first antigen-binding polypeptide (of the CSR) includes a single-chain variable fragment (scFv) having a variable heavy (VH) domain and a variable light (VL) domain, and the VH domain includes the CDR1, CDR2 and CDR3 sequences of the VH domain of GPC2-specific antibody CT3 and the VL domain includes the CDR1, CDR2 and CDR3 sequences of the CT3 VL domain; the second antigen-binding polypeptide (of one polypeptide chain of the AbTCR heterodimer) includes a VH domain and a heavy chain constant region, wherein the VH domain includes the CDR1, CDR2 and CDR3 sequences of the CT3 VH domain; and the third antigen-binding polypeptide (of the second polypeptide chain of the AbTCR heterodimer) includes a VL domain and a light chain constant region, wherein the VL domain includes the CDR1, CDR2 and CDR3 sequences of the CT3 VL domain.

In some aspects in which the AbTCR and CSR are targeted to GPC3, the first antigen-binding polypeptide (of the CSR) includes a scFv that includes a VH domain and a VL domain, and the VH domain includes the CDR1, CDR2 and CDR3 sequences of the VH domain of GPC3-specific antibody hYP7, and the VL domain includes the CDR1, CDR2 and CDR3 sequences of the hYP7 VL domain; the second antigen-binding polypeptide (of one polypeptide chain of the AbTCR heterodimer) includes a VH domain and a heavy chain constant region, wherein the VH domain includes the CDR1, CDR2 and CDR3 sequences of the hYP7 VH domain; and the third antigen-binding polypeptide (of the second polypeptide chain of the AbTCR heterodimer) includes a VL domain and a light chain constant region, wherein the VL domain includes the CDR1, CDR2 and CDR3 sequences of the hYP7 VL domain.

In other aspects in which the AbTCR is targeted to GPC3, the first antigen-binding polypeptide (of the CSR) includes a single-domain antibody having the CDR1, CDR2 and CDR3 sequences of GPC3-specific antibody HN3; the second antigen-binding polypeptide (of one polypeptide chain of the AbTCR heterodimer) includes a VH domain and a heavy chain constant region, wherein the VH domain has the CDR1, CDR2 and CDR3 sequences of the hYP7 VH domain; and the third antigen-binding polypeptide (of the second polypeptide chain of the AbTCR heterodimer) includes a VL domain and a light chain constant region, wherein the VL domain has the CDR1, CDR2 and CDR3 sequences of the hYP7 VL domain.

In other aspects in which the AbTCR and CSR are targeted to GPC3, the first antigen-binding polypeptide (of the CSR) includes a scFv that includes a VH domain and a VL domain, and the VH domain has the CDR1, CDR2 and CDR3 sequences of the hYP7 VH domain and the VL domain has the CDR1, CDR2 and CDR3 sequences of the hYP7 VL domain; the second antigen-binding polypeptide (of one polypeptide chain of the AbTCR heterodimer) includes a single-domain antibody and a heavy chain constant region, wherein the single-domain antibody includes the CDR1, CDR2 and CDR3 sequences of HN3; and the third antigen-binding polypeptide (of the second polypeptide chain of the AbTCR heterodimer) includes a single-domain antibody and a light chain constant region, wherein the single-domain antibody includes the CDR1, CDR2 and CDR3 sequences of HN3.

In other aspects in which the AbTCR and CSR are targeted to GPC3, the first antigen-binding polypeptide (of the CSR) includes a single-domain antibody having the CDR1, CDR2 and CDR3 sequences of HN3; the second antigen-binding polypeptide (of one polypeptide chain of the AbTCR heterodimer) includes a single-domain antibody and a heavy chain constant region, wherein the single-domain antibody has the CDR1, CDR2 and CDR3 sequences of HN3; and the third antigen-binding polypeptide (of the second polypeptide chain of the AbTCR heterodimer) includes a single-domain antibody and a light chain constant region, wherein the single-domain antibody has the CDR1, CDR2 and CDR3 sequences of HN3.

Further provided are isolated immune cells or induced pluripotent stem cells (iPSCs) that express an AbTCR and CSR targeted to GPC2 or GPC3. The immune cells or iPSCs express a first chimeric polypeptide chain that includes a first antigen-binding polypeptide and a co-stimulatory immune cell signaling domain; a second chimeric polypeptide chain that includes a second antigen-binding polypeptide and a first TCR chain or a transmembrane domain-containing fragment thereof; and a third chimeric polypeptide chain that includes a third antigen-binding polypeptide and a second TCR chain or a transmembrane domain-containing fragment thereof. The first, second and third antigen-binding polypeptides specifically bind GPC2, or the first, second and third antigen-binding polypeptides specifically bind GPC3.

In some aspects, the first chimeric polypeptide chain further includes a protein tag or linker (such as a Myc tag).

In some aspects, the co-stimulatory immune cell signaling domain includes all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

In some aspects in which the AbTCR and CSR are targeted to GPC2, the first antigen-binding polypeptide (of the CSR) includes a scFv that includes a VH domain and a VL domain, and the VH domain includes the CDR1, CDR2 and CDR3 sequences of the CT3 VH domain and the VL domain includes the CDR1, CDR2 and CDR3 sequences of the CT3 VL domain; the second antigen-binding polypeptide (of one polypeptide chain of the AbTCR heterodimer) includes a VH domain and a heavy chain constant region, wherein the VH domain includes the CDR1, CDR2 and CDR3 sequences of the CT3 VH domain; and the third antigen-binding polypeptide (of the second polypeptide chain of the AbTCR heterodimer) includes a VL domain and a light chain constant region, wherein the VL domain includes the CDR1, CDR2 and CDR3 sequences of the CT3 VL domain.

In some aspects in which the AbTCR and CSR are targeted to GPC3, the first antigen-binding polypeptide (of the CSR) includes a scFv that includes a VH domain and a VL domain, and the VH domain includes the CDR1, CDR2 and CDR3 sequences of the hYP7 VH domain and the VL domain includes the CDR1, CDR2 and CDR3 sequences of the hYP7 VL domain; the second antigen-binding polypeptide (of one polypeptide chain of the AbTCR heterodimer) includes a VH domain and a heavy chain constant region, wherein the VH domain includes the CDR1, CDR2 and CDR3 sequences of the hYP7 VH domain; and the third antigen-binding polypeptide (of the second polypeptide chain of the AbTCR heterodimer) includes a VL domain and a light chain constant region, wherein the VL domain includes the CDR1, CDR2 and CDR3 sequences of the hYP7 VL domain.

In other aspects in which the AbTCR and CSR are targeted to GPC3, the first antigen-binding polypeptide (of the CSR) includes a single-domain antibody having the CDR1, CDR2 and CDR3 sequences of HN3; the second antigen-binding polypeptide (of one polypeptide chain of the AbTCR heterodimer) includes a VH domain and a heavy chain constant region, wherein the VH domain includes the CDR1, CDR2 and CDR3 sequences of the hYP7 VH domain; and the third antigen-binding polypeptide (of the second polypeptide chain of the AbTCR heterodimer) includes a VL domain and a light chain constant region, wherein the VL domain includes the CDR1, CDR2 and CDR3 sequences of the hYP7 VL domain.

In other aspects in which the AbTCR and CSR are targeted to GPC3, the first antigen-binding polypeptide (of the CSR) includes a scFv having a VH domain and a VL domain, and the VH domain includes the CDR1, CDR2 and CDR3 sequences of the hYP7 VH domain and the VL domain includes the CDR1, CDR2 and CDR3 sequences of the hYP7 VL domain; the second antigen-binding polypeptide (of one polypeptide chain of the AbTCR heterodimer) includes a single-domain antibody and a heavy chain constant region, wherein the single-domain antibody has the CDR1, CDR2 and CDR3 sequences of HN3; and the third antigen-binding polypeptide (of the second polypeptide chain of the AbTCR heterodimer) includes a single-domain antibody and a light chain constant region, wherein the single-domain antibody has the CDR1, CDR2 and CDR3 sequences of HN3.

In other aspects in which the AbTCR and CSR are targeted to GPC3, the first antigen-binding polypeptide (of the CSR) includes a single-domain antibody having the CDR1, CDR2 and CDR3 sequences of HN3; the second antigen-binding polypeptide (of one polypeptide chain of the AbTCR heterodimer) includes a single-domain antibody and a heavy chain constant region, wherein the single-domain antibody has the CDR1, CDR2 and CDR3 sequences of HN3; and the third antigen-binding polypeptide (of the second polypeptide chain of the AbTCR heterodimer) includes a single-domain antibody and a light chain constant region, wherein the single-domain antibody has the CDR1, CDR2 and CDR3 sequences of HN3.

Compositions that include a pharmaceutically acceptable carrier and an immune cell or iPSC expressing an AbTCR and CSR and are also provided.

Further provided are methods of treating a GPC2-positive or GPC3-positive cancer in a subject, or inhibiting tumor growth and/or metastasis of a GPC2-positive or GPC3-positive cancer in a subject, by administering to the subject a therapeutically effective amount of an isolated immune cell or iPSC expressing an AbTCR and CSR, or composition thereof, as disclosed herein.

The foregoing and other features of this disclosure will become more apparent from the following detailed description of several aspects which proceeds with reference to the accompanying figures.

### BRIEF DESCRIPTION OF THE DRAWINGS

FIGS. 1A-1B: Schematics depicting antibody T cell receptors (AbTCRs) and chimeric signaling receptors (CSRs) targeting (FIG. 1A) GPC2 (ART-CT3-CT3 and ART-hCT3-1-hCT3-1) or (FIG. 1B) GPC3 (ART-hYP7-hYP7, ART-hYP7-HN3, ART-HN3-hYP7 and ART-HN3-HN3).

FIGS. 2A-2C: AbTCR+CSR T cells kill GPC2-expressing neuroblastoma cells in vitro. GPC2-targeted AbTCR+CSR T cells were co-cultured with GPC2-positive neuroblastoma IMR5 cells (FIG. 2A), GPC2-knockout (KO) IMR5 cells (FIG. 2B) or GPC2-positive LAN1 cells (FIG. 2C) for 20 hours. Both CT3 and humanized CT3-1 (hCT3-1) AbTCR+CSR T cells potently lysed GPC2-positive IMR5 and LAN1 cells at all tested effector to target (E:T) ratios. Neither of the GPC2-targeted AbTCR+CSR T cells killed GPC2 KO IMR5 cells, demonstrating the antigen specificity of the AbTCR+CSR T cells.

FIGS. 3A-3H: GPC2-targeted AbTCR+CSR T cells regress established tumors in a metastatic neuroblastoma mouse model. (FIG. 3A) Schematic of the study design. IMR5-Luc cells were injected into the tail vein of study animals thirty days prior to infusion of 10 million AbTCR+CSR T cells. Animals were imaged weekly for four weeks. (FIG. 3B) Average bioluminescence of mock-treated mice and mice treated with GPC2-targeted AbTCR+CSR T cells (ART-CT3-CT3 or ART-hCT3-1-hCT3-1). Both CT3 and hCT3-1 AbTCR+CSR T cells rapidly induced regression of established neuroblastoma tumors within the first week post-treatment. (FIGS. 3C-3E) Bioluminescence of individual mock-treated animals (FIG. 3C) or animals treated with CT3 AbTCR+CSR T cells (FIG. 3D) or hCT3-1 AbTCR+CSR T cells (FIG. 3E). (FIG. 3F) Bioluminescent tumor images. Treatment with either CT3 or hCT3-1 AbTCR+CSR T cells completely regressed established neuroblastoma tumors in mice, whereas all mock-treated mice exhibited large tumors. (FIG. 3G) Body weight of mock-treated mice and mice treated with CT3 or hCT3 AbTCR+CSR T cells. Body weights were similar among the three groups. (FIG. 3H) Probability of survival of mock-treated mice and mice treated with CT3 or hCT3 AbTCR+CSR T cells. Treatment with either CT3 or hCT3 AbTCR+CSR T cells improved survival compared to mock-treated animals.

FIG. 4: GPC3 binding of hYP7 and HN3 AbTCR+CSR T cells. Shown is flow cytometric analysis of Jurkat T cells expressing GPC3-targeted CAR (hYP7) and four different GPC3-targeted AbTCR+CSR combinations (hYP7-hYP7, hYP7-HN3, HN3-HN3, and HN3-hYP7). Binding affinity was tested using GPC3 protein concentrations of 0.04 nM and 0.08 nM.

FIGS. 5A-5E: GPC3-targeted AbTCR+CSR T cells induce cytotoxicity against the Hep3B liver cancer cell line. (FIG. 5A) Cytolytic activity of CAR (hYP7) T cells and AbTCR+CSR T cells after 24 or 96 hours of incubation with Hep3B cells in a 2-fold dose dependent manner. (FIGS. 5B-5E) Concentration of cytokines IL-2, IL-4, IL6-, IL-8, IL-10, TNF-α, IFN-γ, and GM-CSF in the supernatants of CAR (hYP7) and AbTCR+CSR (hYP7-hYP7) cytotoxicity assays at an E:T ratio of 5:1 (as in FIG. 5A), measured by ELISA.

FIGS. 6A-6B: Inhibition of Wnt/β-catenin signaling and activation of NFAT signaling by GPC3-targeted CAR and AbTCR+CSR T cells. (FIG. 6A) Western blot showing CAR (hYP7) T cells and AbTCR+CSR T cells suppressed the expression of β-catenin in Hep3B cells after 2, 4, and 8 hours of administration. (FIG. 6B) Fluorescence microscopy images of Hep3B co-cultured with NFAT-Jurkat-tdTomato reporter cells transfected with hYP7 CAR and AbTCR+CSR constructs. AbTCR+CSR (hYP7-hYP7) NFAT-Jurkat cells showed the greatest NFAT signal, followed by AbTCR+CSR (hYP7-HN3) NFAT-Jurkat, AbTCR+CSR (HN3-hYP7) NFAT-Jurkat, AbTCR+CSR (HN3-HN3) NFAT-Jurkat, and CAR (hYP7) NFAT-Jurkat.

FIGS. 7A-7E: Potency of GPC3-targeted CAR and AbTCR+CSR T cells in a Hep3B mouse xenograft model. (FIG. 7A) Schematic of the study design. Hep3B xenograft mice were i.p. infused with 10 million GPC3-targeted CAR (hYP7), AbTCR+CSR (hYP7-hYP7), AbTCR+CSR (hYP7-HN3), AbTCR+CSR (HN3-HN3), or AbTCR+CSR (HN3-hYP7) T cells, or irrelevant CAR (CD19 CAR) T cells 12 days after tumor inoculation with 4 million Hep3B-luc cells. (FIG. 7B) Representative bioluminescence images of Hep3B tumor growth. Mice treated with 10 million AbTCR+CSR (hYP7-hYP7) T cells and AbTCR+CSR (hYP7-HN3) T cells showed better anti-tumor activity than CAR (hYP7), AbTCR+CSR (HN3-HN3), AbTCR+CSR (HN3-hYP7), and CAR (CD19) T cell injection. (FIG. 7C) Tumor bioluminescence as photons per second in CAR and AbTCR+CSR T cell treated mice. (FIG. 7D) Kaplan-Meier survival curve of mice after infusion. All mice administered hYP7-hYP7 T cells or HN3-HN3 T cells survived to the end of the study period. (FIG. 7E) Cytotoxicity of AbTCR+CSR (hYP7-hYP7) T cells was compared to cytotoxicity of AbTCR+CSR (hYP7-hYP7) T cells recovered from mouse spleens 7 weeks after injection. The AbTCR+CSR T cells were co-cultured with Hep3B cells and cytolytic activity was measured 24 hours after co-culture. CD19-specific CAR T cells were used as a negative control.

FIGS. SA-8E: AbTCR+CSR (hYP7-hYP7) T cells exhibit an anti-tumor effect in an orthotopic Hep3B xenograft model. (FIG. 8A) Schematic of the study design. Hep3B orthotopic xenograft mice were i.v. infused with 5 million AbTCR+CSR (hYP7-hYP7) T cells or control CAR (CD19) T cells 35 days after tumor inoculation. Imaging was performed weekly to evaluate tumor size. (FIG. 8B) Representative bioluminescence images of Hep3B tumor growth in the xenograft model. Mice treated with 5 million AbTCR+CSR (hYP7-hYP7) T cells exhibited a significant reduction in tumor size compared to control mice treated with CAR (CD19) T cells. (FIG. 8C) Tumor bioluminescence measured as photons per second. (FIG. 8D) AbTCR+CSR (hYP7-hYP7) T cells isolated from mouse blood expressed lower levels of PD-1 than control CAR (CD19) T cells at week 3 after infusion. (FIG. 8E) In mouse blood, AbTCR+CSR (hYP7-hYP7) T cells showed a higher proportion of effector memory T cells (Tₑₘ) and terminally differentiated effector memory T cells (Tₑₘᵣₐ) than CAR (CD19) T cells.

FIGS. 9A-9D: AbTCR+CSR (hYP7-hYP7) T cells potently reduce Hep3B subcutaneous (s.c.) tumors. (FIG. 9A) Schematic of the study design. Hep3B GL cells (5 million) were subcutaneously injected into NSG mice. Hep3B xenograft mice were i.v. infused with 5 or 2.5 million AbTCR+CSR (hYP7-hYP7), AbTCR+CSR (HN3-HN3), CAR (hYP7), or control CAR (CD19) T cells 21 days after tumor inoculation. (FIG. 9B) Hep3B tumor growth after infusion of 5 million T cells. Mice (N=2) treated with 5 million AbTCR+CSR (hYP7-hYP7) T cells demonstrated tumor regression at week 2. CAR (hYP7) T cells inhibited tumor growth relative to control CD19-targeted CAR T cells, but treatment with AbTCR+CSR (HN3-HN3) T cells was ineffective. (FIG. 9C) Images showing tumor size in treated mice at the end of the study. (FIG. 9D) Mice (N=5) treated with 2.5 million AbTCR+CSR (hYP7-hYP7) T cells exhibited significantly reduced tumor growth relative to mice treated with CD19 CAR T cells.

FIGS. 10A-10F: AbTCR+CSR T cells (hYP7-hYP7 and hYP7-HN3) are more potent than CAR T cells (hYP7) for treating Huh-7 s.c. tumors. (FIG. 10A) Schematic of the study design. Five million Huh-7 GL cells (low GPC3 antigen density) were intraperitoneally (i.p.) injected into NSG mice. Huh-7 xenograft mice were i.v. infused with 5 million control CAR (CD19 CAR), control AbTCR (CD19 AbTCR), GPC3-targeted AbTCR+CSR (hYP7-hYP7 or hYP7-HN3) or GPC3-targeted CAR (hYP7 CAR) T cells 19 days after tumor inoculation. Treated animals were imaged weekly to assess tumor size. (FIG. 10B) Huh7 tumor size measured 7, 14 and 21 days after treatment. Mice treated with 5 million AbTCR+CSR (hYP7-hYP7) or AbTCR+CSR (hYP7-HN3) T cells exhibited tumor regression by week 2. Treatment with CAR (hYP7) T cells did not reduce tumor size compared to control animals treated with CD19 CAR or CD19 AbTCR T cells. (FIG. 10C) Images showing tumor size in treated mice at the end of the study. (FIG. 10D) Absolute number of CAR-T or AbTCR+CSR-T cells in mouse blood at week 3 of treatment. Higher T cell proliferation was elicited by treatment with GPC3 AbTCR+CSR T cells compared to treatment with GPC3 CAR-T cells. AbTCR+CSR (hYP7-hYP7) T cells were more abundant than AbTCR+CSR (hYP7-HN3) T cells in treated mice. (FIG. 10E) A lower level of PD-1 expression was found in ex vivo AbTCR+CSR (hYP7-hYP7) T cells compared to other treatment groups. (FIG. 10F) GPC3-targeted AbTCR+CSR T cells (hYP7-hYP7 and hYP7-HN3) showed a higher proportion of Tₑₘᵣₐ than GPC3-targeted CAR T cells (hYP7).

FIGS. 11A-11C: AbTCR+CSR T cells (hYP7-hYP7 and hYP7-HN3) slowed down the growth of large Huh-7 tumors. (FIG. 11A) Schematic of the study design. Five million Huh-7 GL cells were subcutaneously (s.c.) injected into NSG mice. Huh-7 xenograft mice were i.v. infused with 5 million AbTCR+CSR (hYP7-hYP7), AbTCR+CSR (hYP7-HN3), or control AbTCR (CD19 AbTCR) T cells 30 days after tumor inoculation. (FIG. 11B) Huh7 tumor volume at Day 0 and Day 14 after T cell infusion. Huh-7 tumor size on Day 0 was large (average volume of approximately 500 mm³). On Day 14, the tumor size in mice treated with irrelevant AbTCR T cells (CD19 AbTCR) increased to 7,344 mm³ per mouse on average. The hYP7-hYP7 AbTCR+CSR T cells significantly slowed down tumor growth (average tumor size of 3,358 mm³), while the hYP7-HN3 AbTCR+CSR T cells moderately slowed down tumor growth (average tumor size around 5,996 mm³). (FIG. 11C) Hematoxylin and eosin (H&E) and immunohistochemistry (IHC) staining of Huh-7 tumors isolated from the Huh-7 s.c. mouse model. Two markers, GPC3 and CD3, were used to do the IHC staining. After 10 days of the AbTCR+CSR-T cell treatment, tumor-infiltrated hYP7-hYP7 (ART-Y-Y-myc) T cells were found in Huh-7 tumors.

FIG. 12: GPC2 binding of CT3 and hCT3 CARs and AbTCRs. Shown is the flow cytometric analysis of Jurkat T cells expressing CT3 or hCT3 CARs (CT3-CD28HTM and hCT3-CD28HTM, respectively) or AbTCRs+CSRs (ART-CT3-CT3 and ART-hCT3-1-hCT3-1, respectively). Mock T cells not expressing a CAR or AbTCR+CSR were included as a control.

FIGS. 13A-13F: Potency of GPC2-targeted CAR and AbTCR+CSR T cells in an IMR-5 mouse xenograft model. (FIG. 13A) Schematic of the study design. IMR-5 xenograft mice were i.v. infused with 5 million CAR (CT3-CD28HTM and hCT3-CD28HTM), AbTCR+CSR (ART-CT3-CT3 and ART-hCT3-hCT3) or mock T cells 28 days after tumor inoculation with IMR-5-luc cells. (FIG. 13B) Representative bioluminescence images of IMR-5 tumor growth. Treatment with 5 million AbTCR+CSR (ART-CT3-CT3 and ART-hCT3-hCT3) T cells showed better anti-tumor activity than treatment with CAR (CT3-CD28HTM and hCT3-CD28HTM) T cells. ART-hCT3-hCT3 was the most potent in this study. (FIG. 13C) Tumor bioluminescence as photons per second in CAR and AbTCR+CSR T cell treated mice. (FIG. 13D) Kaplan-Meier survival curve of mice after infusion. (FIG. 13E) Representative bioluminescence images of IMR-5 tumor rechallenge. For the tumor rechallenge experiment, luciferase-expressing IMR-5 tumor cells were inoculated i.v. into three mice after 32 days of treatment with ART-hCT3-hCT3 (in FIG. 13B, each star symbol indicates a mouse that was rechallenged). These mice remained tumor-free 5 weeks after tumor rechallenge while all of the untreated mice showed growth of IMR-5 tumors. (FIG. 13F) Tumor bioluminescence as photons per second in tumor IMR5-luc rechallenged mice after the treatment of ART-hCT3-hCT3, as compared to mice that were untreated with any T cells and directly inoculated with IMR-5 tumor cells.

### SEQUENCE LISTING

The nucleic acid and amino acid sequences listed in the accompanying sequence listing are shown using standard letter abbreviations for nucleotide bases and single letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the amino acid sequence of the ART-CT3-CT3 AbTCR+CSR.

SEQ ID NO: 2 is the amino acid sequence of the ART-hCT3-1-hCT3-1 AbTCR+CSR

SEQ ID NO: 3 is the amino acid sequence of the ART-hYP7-hYP7 AbTCR+CSR.

SEQ ID NO: 4 is the amino acid sequence of the ART-hYP7-HN3 AbTCR+CSR.

SEQ ID NO: 5 is the amino acid sequence of the ART-HN3-hYP7 AbTCR+CSR.

SEQ ID NO: 6 is the amino acid sequence of the ART-HN3-HN3 AbTCR+CSR.

SEQ ID NO: 7 is the amino acid sequence of the CT3 VH domain.

SEQ ID NO: 8 is the amino acid sequence of the CT3 VL domain.

SEQ ID NO: 9 is the amino acid sequence of the CT3 (VL-VH) scFv.

SEQ ID NO: 10 is the amino acid sequence of the hCT3-1 VH domain.

SEQ ID NO: 11 is the amino acid sequence of the hCT3-1 VL domain.

SEQ ID NO: 12 is the amino acid sequence of the hCT3-1 (VL-VH) scFv.

SEQ ID NO: 13 is the amino acid sequence of the hYP7 VH domain.

SEQ ID NO: 14 is the amino acid sequence of the hYP7 VL domain.

SEQ ID NO: 15 is the amino acid sequence of the hYP7 (VL-VH) scFv.

SEQ ID NO: 16 is the amino acid sequence of single-domain antibody HN3.

SEQ ID NO: 17 is the amino acid sequence of the human IgG1 CH1 domain.

SEQ ID NO: 18 is the amino acid sequence of the human IgG kappa constant domain.

SEQ ID NO: 19 is the amino acid sequence of a TCR delta chain fragment.

SEQ ID NO: 20 is the amino acid sequence of a TCR gamma chain fragment.

SEQ ID NO: 21 is the amino acid sequence of a human CD30 fragment that includes its transmembrane and intracellular domains.

SEQ ID NO: 22 is the amino acid sequence of the mouse IgG kappa signal peptide.

SEQ ID NO: 23 is the amino acid sequence of a c-Myc tag.

SEQ ID NO: 24 is the amino acid sequence of a P2A peptide.

SEQ ID NO: 25 is the amino acid sequence of a T2A peptide.

SEQ ID NO: 26 is a nucleic acid sequence encoding the CT3 VH domain.

SEQ ID NO: 27 is a nucleic acid sequence encoding the CT3 VL domain.

SEQ ID NO: 28 is a nucleic acid sequence encoding the hCT3-1 VH domain.

SEQ ID NO: 29 is a nucleic acid sequence encoding the hCT3-1 VL domain.

SEQ ID NO: 30 is a nucleic acid sequence encoding the hYP7 VH domain.

SEQ ID NO: 31 is a nucleic acid sequence encoding the hYP7 VL domain.

SEQ ID NO: 32 is a nucleic acid sequence encoding single-domain antibody HN3.

SEQ ID NO: 33 is a nucleic acid sequence encoding a CD28 fragment that includes TM and intracellular signaling domains.

SEQ ID NO: 34 is a nucleic acid sequence encoding a CD28 fragment that includes an intracellular signaling domain.

SEQ ID NO: 35 is a nucleic acid sequence encoding a 4-1BB fragment that includes TM and intracellular signaling domains.

SEQ ID NO: 36 is a nucleic acid sequence encoding a 4-1BB fragment that includes an intracellular signaling domain.

SEQ ID NO: 37 is a nucleic acid sequence encoding a CD30 fragment that includes TM and intracellular signaling domains.

SEQ ID NO: 38 is a nucleic acid sequence encoding a CD30 fragment that includes an intracellular signaling domain.

SEQ ID NO: 39 is a nucleic acid sequence encoding a partial constant region and the TM and intracellular domains of TCRα.

SEQ ID NO: 40 is a nucleic acid sequence encoding a partial constant region and the TM and intracellular domains of TCRβ.

SEQ ID NO: 41 is a nucleic acid sequence encoding a partial constant region and the TM and intracellular domains of TCRδ.

SEQ ID NO: 42 is a nucleic acid sequence encoding the TM domain of TCRδ.

SEQ ID NO: 43 is a nucleic acid sequence encoding a partial constant region and the TM and intracellular domains of TCRγ.

SEQ ID NO: 44 is a nucleic acid sequence encoding the TM domain of TCRγ.

SEQ ID NO: 45 is a nucleic acid sequence encoding the IgG1 CH1 domain.

SEQ ID NO: 46 is a nucleic acid sequence encoding a light chain constant region.

SEQ ID NO: 47 is the amino acid sequence of a partial constant region and the TM and intracellular domains of TCRα.

SEQ ID NO: 48 is the amino acid sequence of a partial constant region and the TM and intracellular domains of TCRβ.

SEQ ID NO: 49 is the amino acid sequence of the CD30 intracellular signaling domain.

SEQ ID NO: 50 is the amino acid sequence of the CD28 TM and intracellular signaling domains.

SEQ ID NO: 51 is the amino acid sequence of the CD28 intracellular signaling domain.

SEQ ID NO: 52 is the amino acid sequence of the 4-1BB TM and intracellular signaling domains.

SEQ ID NO: 53 is the amino acid sequence of the 4-1BB intracellular signaling domain.

### DETAILED DESCRIPTION

### I. Abbreviations

AbTCR antibody T cell receptor
ALL acute lymphoblastic leukemia
ARMS alveolar rhabdomyosarcoma
CAR chimeric antigen receptor
CDR complementarity determining region
CNS central nervous system
CSR chimeric signaling receptor
DC dendritic cell
DRCT desmoplastic small round cell tumor
ERMS embryonal rhabdomyosarcoma
GPC2 glypican-2
GPC3 glypican-3
H&E hematoxylin and cosin
HCC hepatocellular carcinoma
IHC immunohistochemistry
i.p. intraperitoneal
i.v. intravenous
KO knockout
MAIT mucosal-associated invariant T
NK natural killer
NSG NOD-SCID-Gamma
RMS rhabdomyosarcoma
s.c. subcutaneous
scFv single-chain variable fragment
T_{cm} central memory T cell
Tₑₘ effector memory T cell
Tₑₘᵣₐ effector memory T cell re-expressing CD45RA
T_{scm} stem cell memory T cell
TCR T cell receptor
TM transmembrane
VH variable heavy
VL variable light

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Acute lymphoblastic leukemia (ALL): An acute form of leukemia characterized by the overproduction of lymphoblasts. ALL is most common in childhood, peaking at ages 2-5.

Administration: To provide or give a subject an agent, such as an AbTCR+CSR-expressing cell provided herein, by any effective route. Exemplary routes of administration include, but are not limited to, injection (such as subcutaneous, intramuscular, intradermal, intraperitoneal, intracerebral, intraventricular, intracranial, intramedullar, intravenous, intra-arterial (including hepatic intra-arterial), intraosseous, intravitreal, and intratumoral), infusion, oral sublingual, rectal, transdermal, intranasal, vaginal and inhalation routes.

Antibody: A polypeptide ligand that includes at least one variable region that recognizes and binds (such as specifically recognizes and specifically binds) an epitope of an antigen, such as GPC2 or GPC3. Mammalian immunoglobulin molecules are composed of a heavy (H) chain and a light (L) chain, each of which has a variable region, termed the variable heavy (VH) region and the variable light (VL) region, respectively. Together, the VH region and the VL region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8: e1002388, 2012; and Kunik et al., Nucleic Acids Res 40: W521-524, 2012; “Paratomc CDRs”), MacCallum et al. (J. Mol. Biol. 262:732-745, 1996), Honegger and Plückthun (J. Mol. Biol., 309:657-670. 2001), and the ImMunoGeneTics (IMGT) database (see, Lefranc, Nucleic Acids Res 29:207-9, 2001; the “IMGT” numbering scheme). The Kabat, Paratome and IMGT databases are maintained online. These definitions of antibody CDRs include overlapping or subsets of amino acid residues when compared against each other. Nevertheless, application of any of the above-referenced definitions to refer to a CDR of an antibody or an antibody-derived antigen-binding polypeptide is intended to be within the scope of the term as defined and used herein.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by known methods. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they are substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antigen-binding polypeptide: A polypeptide having at least one antigen-binding domain that confers antigen-specific binding to the polypeptide. In some aspects herein, the antigen-binding polypeptide includes a VH domain (such as a CT3, hCT3 or hYP7 VH domain), a VL domain (such as a CT3, hCT3 or hYP7 VL domain), a single-domain antibody (e.g., single-domain antibody HN3), or a scFv (such as CT3, hCT3 or hYP7 scFv). In some examples, the antigen-binding polypeptide further includes a constant domain, such as a heavy chain constant region or a light chain constant region.

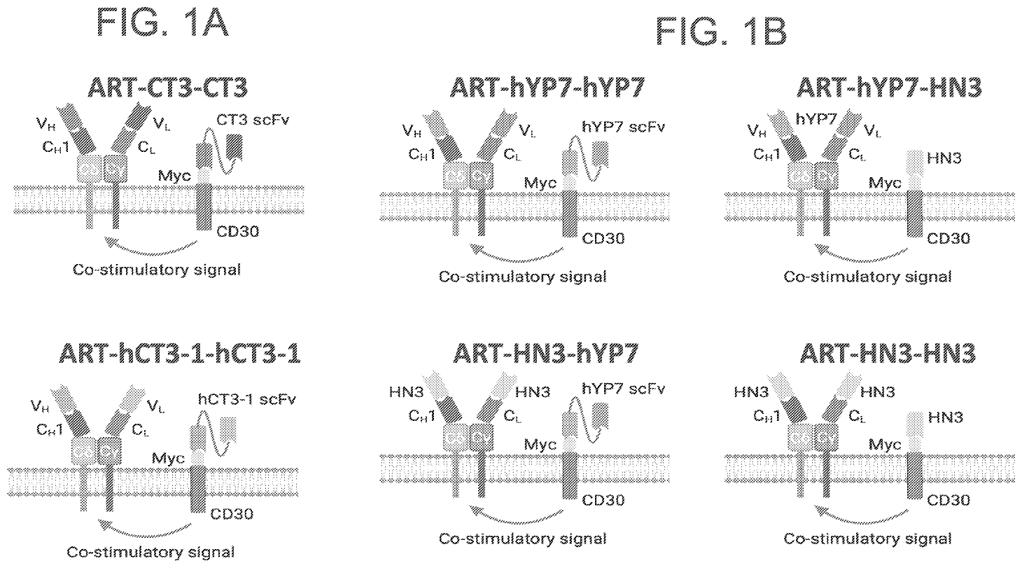

Antibody T cell receptor (AbTCR): A chimeric molecule comprising one or more antibody-derived antigen-binding domains and TCR signaling domains, such as two TCR chains (e.g., alpha, beta, gamma or delta chains) or transmembrane domain-containing fragments thereof. AbTCRs are heterodimers that can be covalently or non-covalently associated (see, e.g., WO 2018/200583). Specific non-limiting examples of AbTCRs and AbTCRs/CSRs are shown in FIGS. 1A-1B.

Binding affinity: Affinity of an antibody or other antigen-binding molecule for an antigen, such as GPC2 or GPC3. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance assays using a BIACORE-2000 or a BIACORE-3000 (BIAcorc, Inc., Piscataway, N.J.). In other aspects, antibody affinity is measured by flow cytometry. An antibody, CAR, AbTCR or CSR that “specifically binds” an antigen (such as GPC2 or GPC3) is an antibody, CAR, AbTCR or CSR that binds the antigen with high affinity and does not significantly bind other unrelated antigens.

CD30: A T cell co-stimulatory protein that is part of the tumor necrosis factor receptor superfamily. CD30, also known as TNFRSF8, is expressed on activated T cells, NK cells and B cells. An exemplary amino acid sequence for CD30 is set forth herein as SEQ ID NO: 21.

Chemotherapeutic agent: Any chemical agent with therapeutic usefulness in the treatment of diseases characterized by abnormal cell growth. Such diseases include tumors, neoplasms, and cancer. In one aspect, a chemotherapeutic agent is an agent of use in treating a GPC2- or GPC3-positive tumor. In one aspect, a chemotherapeutic agent is a radioactive compound. Exemplary chemotherapeutic agents that can be used with the methods provided herein are disclosed in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). In one example, a chemotherapeutic agent is a biologic, such as a therapeutic antibody (e.g., therapeutic monoclonal antibody), such as an anti-GPC2 or anti-GPC3 antibody, as well as other anti-cancer antibodies, such as anti-PD1 or anti-PDL1 (e.g., pembrolizumab and nivolumab), anti-CTLA4 (e.g., ipilimumab), anti-EGFR (e.g., cetuximab), anti-VEGF (e.g., bevacizumab), or combinations thereof (e.g., anti-PD-1 and anti-CTLA-4). Combination chemotherapy is the administration of more than one agent to treat cancer. One example is the administration of GPC2-targeted (or GPC3-targeted) AbTCR+CSR-expressing cells used in combination with a radioactive, biological, or chemical compound, or combinations thereof.

Chimeric antigen receptor (CAR): A chimeric molecule that includes an antigen-binding portion (such as single-domain antibody or scFv) and a signaling domain, such as a signaling domain from a T cell receptor-associated signaling molecule (for example, CD3ζ). In many instances, CARs include an antigen-binding moiety, a hinge region, a transmembrane domain and an endodomain. The endodomain can include a signaling chain having an immunoreceptor tyrosine-based activation motif (ITAM), such as CD3ζ or FcεRIγ. In some cases, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10.

Chimeric signaling receptor (CSR): A chimeric polypeptide chain that includes an antigen-binding polypeptide (such as an antibody or antibody fragment that specifically binds GPC2 or GPC3) and a co-stimulatory signaling domain capable of providing a stimulatory signal to an immune cell (such as a CD30 signaling domain). In some aspects, the co-stimulatory domain includes all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs (CDR1, CDR2 and CDR3).

Conservative variant: In the context of the present disclosure, “conservative” amino acid substitutions are those substitutions that do not substantially affect or decrease the affinity of a protein, such as an antibody, to GPC2 or GPC3. As one example, a monoclonal antibody that specifically binds GPC2 can include at most about 1, at most about 2, at most about 5, and most about 10, or at most about 15 conservative substitutions and specifically bind the GPC2 polypeptide. The term “conservative variant” also includes the use of a substituted amino acid in place of an unsubstituted parent amino acid, provided that the variant retains activity. Non-conservative substitutions are those that reduce an activity (such as affinity) of a protein.

Conservative amino acid substitution tables providing functionally similar amino acids are well known. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects herein, provided are amino acid sequences comprising no more than 10, no more than 9, no more than 8, no more than 7, no more than 6, no more than 5, no more than 4, no more than 3, no more than 2 or no more than 1 amino acid substitutions relative to any amino acid sequence disclosed herein.

Contacting: Placement in direct physical association; includes both in solid and liquid form.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Desmoplastic small round cell tumor (DRCT): A soft tissue sarcoma that predominantly occurs in childhood, particularly in boys. DRCT is an aggressive and rare type of cancer that primarily occurs as masses in the abdomen, but can also be found in the lymph nodes, the lining of the abdomen, diaphragm, spleen, liver, chest wall, skull, spinal cord, intestine, bladder, brain, lungs, testicles, ovaries and the pelvis.

Epitope: An antigenic determinant. These are particular chemical groups or peptide sequences on a molecule that are antigenic (that elicit a specific immune response). An antibody specifically binds a particular antigenic epitope on a polypeptide.

Framework region: Amino acid sequences interposed between CDRs. Framework regions include variable light and variable heavy framework regions. The framework regions serve to hold the CDRs in an appropriate orientation for antigen binding.

Glioma: A type of tumor that occurs in the brain and spinal cord. Gliomas originate in the glial cells that surround and support neurons in the brain, including astrocytes, oligodendrocytes and ependymal cells. There are three classes of gliomas, based on the type of cells from which the tumor arises: astrocytoma, ependymoma, and oligodendroglioma.

Glypican-2 (GPC2): A member of the six-member glypican family of heparan sulfate (HS) proteoglycans that are attached to the cell surface by a GPI anchor (Li et al., Trends Cancer 4 (11): 741-754, 2018). GPC2 mRNA is highly expressed in neuroblastoma and other pediatric cancers (Orentas et al., Front Oncol 2:194, 2012). GPC2 protein is highly expressed in about half of neuroblastoma cases and the high GPC2 expression correlates with poor overall survival compared with patients with low GPC2 expression (Li et al., Proc Natl Acad Sci USA 114 (32): E6623-E6631, 2017). GPC2 is also known as cerebroglycan proteoglycan and glypican proteoglycan 2. GPC2 genomic, mRNA and protein sequences are publicly available (see, for example, NCBI Gene ID 221914).

GPC2-positive cancer: A cancer that expresses or overexpresses GPC2. Examples of GPC2-positive cancers include, but are not limited to, neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma, small-cell lung cancer, or osteosarcoma.

Glypican-3 (GPC3): A member of the glypican family of HS proteoglycans that are attached to the cell surface by a glycosylphosphatidylinositol anchor (Filmus and Selleck, J Clin Invest 108:497-501, 2001). The GPC3 gene codes for a core protein of approximately 70 kD, which can be cleaved by furin to produce an N-terminal 40 kD fragment and a C-terminal 30 kD fragment. Two HS chains are attached on the C-terminal portion of GPC3. GPC3 and other glypican family proteins play a role in cell division and cell growth regulation. GPC3 is highly expressed in HCC and some other human cancers including melanoma, squamous cell carcinomas of the lung, and clear cell carcinomas of the ovary (Ho and Kim, Eur J Cancer 47 (3): 333-338, 2011), but is not expressed in normal tissues. GPC3 is also known as SGB, DGSX, MXR7, SDYS, SGBS, OCI-5, SGBS1 and GTR2-2. There are four known isoforms of human GPC3 (isoforms 1-4) (Ho and Kim, Eur J Cancer 47 (3): 333-338, 2011). Nucleic acid and amino acid sequences of the four isoforms of GPC3 are known, including GenBank Accession numbers: NM_001164617 and NP_001158089 (isoform 1); NM_004484 and NP_004475 (isoform 2); NM_001164618 and NP_001158090 (isoform 3); and NM_001164619 and NP_001158091 (isoform 4).

GPC3-positive cancer: A cancer that expresses or overexpresses GPC3. Examples of GPC3-positive cancers include, but are not limited to, HCC, melanoma, ovarian clear-cell carcinomas, yolk sac tumors (YST), hepatoblastoma, Wilms' tumors, squamous cell carcinoma of the lung, testicular nonseminomatous germ cell tumors, liposarcoma, cervical intraepithelial neoplasia, adenoma of the adrenal gland, schwannoma, salivary gland cancer, glioblastoma, choriocarcinoma, rhabdosarcoma, renal clear-cell carcinoma, and embryonal tumor (Ho and Kim, Eur J Cancer 47 (3): 333-338, 2011; Baumhoer et al., Am J Clin Pathol 129 (6): 899-906, 2008; Saikali and Sinnett, Int J Cancer 89 (5): 418-422, 2000).

Hepatocellular carcinoma (HCC): A primary malignancy of the liver typically occurring in patients with inflammatory livers resulting from viral hepatitis, liver toxins or hepatic cirrhosis (often caused by alcoholism). HCC is also called malignant hepatoma.

Heterologous: Originating from a separate genetic source or species.

Host cells: Cells in which a vector can be propagated and its DNA expressed. The cell may be prokaryotic or eukaryotic. In some examples, the prokaryotic cell is an E. coli cell. In some examples, the eukaryotic cell is a mammalian cell, such as human cell. The human cell can be, for example, a human immune cell such as a T cell (e.g., a cytotoxic T cell, αβT cell, &T cell, or regulatory T cell), natural killer (NK) cell, NK T cell, mucosal-associated invariant T (MAIT) cell, dendritic cell (DC), macrophage or B cell. In other examples, the host cell is an iPSC. The term “host cell” also includes any progeny of the subject host cell. It is understood that all progenies may not be identical to the parental cell since there may be mutations that occur during replication. However, such progenies are included when the term “host cell” is used.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4⁺ response or a CD8⁺ response. In another aspect, the response is a B cell response, and results in the production of specific antibodies.

Isolated: An “isolated” biological component, such as a nucleic acid, protein (including antibodies) or organelle, has been substantially separated or purified away from other biological components in the environment (such as a cell) in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antigen-binding fragment (such as an scFv fragment) which serves to indirectly bond the heavy chain variable region to the light chain variable region. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Mammal: This term includes both human and non-human mammals. Similarly, the term “subject” includes both human and veterinary subjects, such as mice, rats, cows, cats, dogs, pigs, and non-human primates.

Medulloblastoma: A fast-growing type of cancer that forms in the cerebellum. Medulloblastomas tend to spread through the cerebrospinal fluid to the spinal cord or to other parts of the brain. They may also spread to other parts of the body, but this is rare. Medulloblastomas are most common in children and young adults. They are a type of central nervous system embryonal tumor.

Melanoma: A form of cancer that originates in melanocytes (cells that make the pigment melanin). Melanocytes are found primarily in the skin, but are also present in the bowel and eye. Melanoma in the skin includes superficial spreading melanoma, nodular melanoma, acral lentiginous melanoma, and lentigo maligna (melanoma). Any of the above types may produce melanin or can be amelanotic. Similarly, any subtype may show desmoplasia (dense fibrous reaction with neurotropism) which is a marker of aggressive behavior and a tendency to local recurrence. Other melanomas include clear cell sarcoma, mucosal melanoma and uveal melanoma.

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Neuroblastoma: A solid tumor arising from embryonic neural crest cells. Neuroblastoma commonly arises in and around the adrenal glands, but can occur anywhere that sympathetic neural tissue is found, such as in the abdomen, chest, neck or nerve tissue near the spine. Neuroblastoma typically occurs in children younger than 5 years of age.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Osteosarcoma: A type of cancerous tumor found in the bone. Osteosarcoma is an aggressive cancer arising from primitive transformed cells of mesenchymal origin. This type of cancer is most prevalent in children and young adults.

Ovarian cancer: Cancer that forms in tissues of the ovary (one of a pair of female reproductive glands in which the ova, or eggs, are formed). Most ovarian cancers are either ovarian epithelial carcinomas (cancer that begins in the cells on the surface of the ovary) or malignant germ cell tumors (cancer that begins in egg cells).

Ovarian clear cell carcinoma: A distinct histopathologic subtype of epithelial ovarian cancer with an incidence of less than 5% of all ovarian malignancies. When viewed under a microscope, the insides of the cells of this type of tumor appear clear.

Pediatric cancer: A cancer that develops in children ages 0 to 14. The major types of pediatric cancers include, for example, neuroblastoma, acute lymphoblastic leukemia (ALL), embryonal rhabdomyosarcoma (ERMS), alveolar rhabdomyosarcoma (ARMS), Ewing's sarcoma, desmoplastic small round cell tumor (DRCT), osteosarcoma, brain and other CNS tumors (such as medulloblastoma), Wilm's tumor, non-Hodgkin lymphoma, and retinoblastoma.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the AbTCR+CSR-expressing cells and other compositions disclosed herein. The nature of the carrier can depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating, or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number or size of metastases. “Ameliorating” refers to the reduction in the number or severity of signs or symptoms of a disease, such as cancer.

Purified: The term purified does not require absolute purity; rather, it is intended as a relative term. Thus, for example, a purified peptide preparation is one in which the peptide or protein is more enriched than the peptide or protein is in its natural environment within a cell. Similarly, a purified cell is one in which the cell is more enriched than the cell is in its natural environment within a subject, or in which the cell is substantially free of other cell types. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95%, 98%, 99%, 99.9%, or 99.99% pure. Thus, in one specific, non-limiting example, a substantially purified protein is at least 90% free of other proteins or cellular components. A substantially purified cell (such as a cell expressing an AbTCR+CSR provided herein) can be at least 90%, 95%, 98%, 99%, 99.9%, or 99.99% pure. Thus, in one specific, non-limiting example, a substantially purified cell expressing an AbTCR+CSR provided herein is at least 99% free of other cells (such as other immune cells or other cells not expressing an AbTCR+CSR provided herein) or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Retinoblastoma: A type of cancer that forms in the tissues of the retina. Retinoblastoma usually occurs in children younger than 5 years of age. It may be hereditary or nonhereditary (sporadic).

Rhabdomyosarcoma (RMS): A soft tissue malignant tumor of skeletal muscle origin. The most common primary sites for rhabdomyosarcoma are the head and neck (e.g., parameningeal, orbit, pharyngeal, etc.), the genitourinary tract, and the extremities. Other less common primary sites include the trunk, chest wall, the abdomen (including the retroperitoneum and biliary tract), and the perineal/anal region. There are at least two types of RMS; the most common forms are alveolar RMS (ARMS) and embryonal histological RMS (ERMS). Approximately 20% of children with rhabdomyosarcoma have the ARMS subtype. An increased frequency of this subtype is noted in adolescents and in patients with primary sites involving the extremities, trunk, and perineum/perianal region. ARMS is associated with chromosomal translocations encoding a fusion gene involving FKHR on chromosome 13 and members of the PAX family. The embryonal subtype is the most frequently observed subtype in children, accounting for approximately 60-70% of rhabdomyosarcomas of childhood. Tumors with embryonal histology typically arise in the head and neck region or in the genitourinary tract, although they may occur at any primary site. ERMS is characterized by a younger age at diagnosis, loss of heterozygosity, and altered genomic imprinting.

Self-cleaving peptides: Peptides that induce the ribosome to skip the synthesis of a peptide bond at the C-terminus, leading to separation of the peptide sequence and a downstream polypeptide. Virally encoded 2A peptides are a type of self-cleaving peptide. Virally encoded 2A peptides include, for example, 2A peptides from porcine teschovirus-1 (PTV1; P2A), foot and mouth disease virus (FMDV; F2A), equine rhinitis A virus (ERAV; E2A) and Thosea asigna virus (TaV; T2A).

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody or AbTCR and/or CSR that specifically binds GPC2 or GPC3 are typically characterized by possession of at least about 75%, for example at least about 80%, 90%, 95%, 96%, 97%, 98% or 99% sequence identity counted over the full-length alignment with the amino acid sequence of the antibody or CAR using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95% depending on their similarity to the reference sequence. Methods for determining sequence identity over such short windows are available at the NCBI website. These sequence identity ranges are provided for guidance only; it is possible that strongly significant homologs could be obtained that fall outside of the ranges provided.

Squamous cell carcinoma: A type of cancer that originates in squamous cells, thin, flat cells that form the surface of the skin, eyes, various internal organs, and the lining of hollow organs and ducts of some glands. Squamous cell carcinoma is also referred to as epidermoid carcinoma. One type of squamous cell carcinoma is squamous cell carcinoma of the lung. Squamous cell carcinoma is the most common type of skin cancer.

Subject: Living multi-cellular vertebrate organisms, a category that includes both human and veterinary subjects, including human and non-human mammals such as pigs, mice, rats, rabbits, sheep, horses, cows, dogs, cats and non-human primates.

Synthetic: Produced by artificial means in a laboratory, for example a synthetic nucleic acid or protein (for example, an antibody) can be chemically synthesized in a laboratory.

T cell receptor (TCR): A protein complex expressed on T lymphocytes. TCRs are heterodimeric receptors composed of αβ or γδ chains that pair on the surface of a T cell. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.). Amino acid sequences of exemplary TCR delta chain and gamma chain fragments that include a transmembrane domain are set forth herein as SEQ ID NO: 19 and SEQ ID NO: 20, respectively.

Therapeutically effective amount: A quantity of a specific substance sufficient to achieve a desired effect in a subject being treated. For instance, this can be the amount necessary to inhibit or suppress growth of a tumor. In one aspect, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment. When administered to a subject, a dosage will generally be used that will achieve target tissue concentrations (for example, in tumors) that has been shown to achieve a desired in vitro effect.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some examples, the vector is a viral vector, such as a lentiviral vector, an adenovirus vector, or an adeno-associated virus (AAV) vector.

### III. Nucleic Acid Molecules and Vectors Encoding AbTCRs and CSRs

Described herein are nucleic acid molecules encoding, and immune cells expressing, antibody TCRs (AbTCRs) and chimeric signaling receptors (CSRs) that include antigen-binding domains specific for either GPC2 or GPC3. It is demonstrated herein that in multiple different xenograft models, immune cells expressing the disclosed recombinant AbTCRs and CSRs effectively treated GPC2-positive or GPC3-positive tumors, and were significantly more potent than similarly targeted CAR T cells.

Provided herein are nucleic acid molecules (or sets of nucleic acid molecules) encoding an AbTCR and/or CSR targeted to either GPC2 or GPC3. The disclosed nucleic acid molecules or sets of nucleic acid molecules include a first module that includes a nucleic acid sequence encoding a first signal peptide, a nucleic acid sequence encoding a first antigen-binding polypeptide, and a nucleic acid sequence encoding a co-stimulatory immune cell signaling domain; a second module that includes a nucleic acid sequence encoding a second signal peptide, a nucleic acid sequence encoding a second antigen-binding polypeptide, and a nucleic acid sequence encoding a first T cell receptor (TCR) chain or a transmembrane domain-containing fragment thereof; and a third module that includes a nucleic acid sequence encoding a third signal peptide, a nucleic acid sequence encoding a third antigen-binding polypeptide, and a nucleic acid sequence encoding a second TCR chain or a transmembrane domain-containing fragment thereof. In the disclosed AbTCR+CSR, the first, second and third antigen-binding polypeptides specifically bind GPC2, or the first, second and third antigen-binding polypeptides specifically bind GPC3.

In some aspects, a single nucleic acid molecule includes the first module, the second module and the third module, thus a single nucleic acid molecule encodes both the AbTCR and CSR. In other aspects, the set of nucleic acid molecules includes a first nucleic acid molecule that includes the first module and a second nucleic acid molecule that includes the second module and the third module. In these aspects, the first nucleic acid molecule encodes the CSR and the second nucleic acid molecule encodes both chains of the AbTCR. In yet other aspects, the set of nucleic acid molecules includes a first nucleic acid molecule that includes the first module, a second nucleic acid molecule that includes the second module, and a third nucleic acid molecule that includes the third module. In these aspects, the first nucleic acid molecule encodes the CSR, the second nucleic acid molecule encodes one chain of the AbTCR and the third nucleic acid molecule encodes the second chain of the AbTCR.

In some aspects, the first module further includes a nucleic acid sequence encoding a protein tag or linker. In specific non-limiting examples, the protein tag is a Myc tag, such as the Myc tag having the amino acid sequence of SEQ ID NO: 23.

In some aspects, the co-stimulatory immune cell signaling domain includes all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

In some aspects, the first TCR chain or the transmembrane domain-containing fragment thereof is derived from a TCR γ chain, and the second TCR chain or the transmembrane domain-containing fragment thereof is derived from a TCR δ chain; or the first TCR chain or the transmembrane domain-containing fragment thereof is derived from a TCR δ chain, and the second TCR chain or the transmembrane domain-containing fragment thereof is derived from a TCR γ chain. In other aspects, the first TCR chain or the transmembrane domain-containing fragment thereof is derived from a TCR α chain, and the second TCR chain or the transmembrane domain-containing fragment thereof is derived from a TCR β chain; or the first TCR chain or the transmembrane domain-containing fragment thereof is derived from a TCR β chain, and the second TCR chain or the transmembrane domain-containing fragment thereof is derived from a TCR α chain.

In some aspects in which the AbTCR+CSR is targeted to GPC2, the first antigen-binding polypeptide includes a single-chain variable fragment (scFv) having a variable heavy (VH) domain and a variable light (VL) domain, and the VH domain includes the CDR1, CDR2 and CDR3 sequences of the VH domain of GPC2-specific antibody CT3 (SEQ ID NO: 7) or hCT3-1 (SEQ ID NO: 10) and the VL domain includes the CDR1, CDR2 and CDR3 sequences of the CT3 (SEQ ID NO: 8) or hCT3-1 VL domain (SEQ ID NO: 11); the second antigen-binding polypeptide includes a VH domain and a heavy chain constant region, wherein the VH domain includes the CDR1, CDR2 and CDR3 sequences of the CT3 (SEQ ID NO: 7) or hCT3-1 VH domain (SEQ ID NO: 10); and the third antigen-binding polypeptide includes a VL domain and a light chain constant region, wherein the VL domain includes the CDR1, CDR2 and CDR3 sequences of the CT3 (SEQ ID NO: 8) or hCT3-1 VL domain (SEQ ID NO: 11).

In some examples, the CT3/hCT3-1 VH domain CDR1, CDR2 and CDR3 sequences respectively include residues 31-35, 50-66 and 99-112 of SEQ ID NO: 7, or residues 26-33, 51-58 and 97-112 of SEQ ID NO: 7. In some examples, the CT3/hCT3-1 VL domain CDR1, CDR2 and CDR3 sequences respectively include residues 24-33, 49-55 and 88-96 of SEQ ID NO: 8, or residues 27-31, 49-51 and 88-96 of SEQ ID NO: 8.

In some examples, the amino acid sequence of the VH domain is at least 85%, at least 90%, at least 95%, at least 96%, at least 97%, at least 98%, or at least 99% identical to SEQ ID NO: 7 and the amino acid sequence of the VL domain is at least 85%, at least 90%, at least 95%, at least 96%, at least 97%, at least 98%, or at least 99% identical to SEQ ID NO: 8. In other examples, the amino acid sequence of the VH domain is at least 85%, at least 90%, at least 95%, at least 96%, at least 97%, at least 98%, or at least 99% identical to SEQ ID NO: 10 and the amino acid sequence of the VL domain is at least 85%, at least 90%, at least 95%, at least 96%, at least 97%, at least 98%, or at least 99% identical to SEQ ID NO: 11. In some examples, the amino acid sequence of the scFv of the first antigen-binding polypeptide is at least 85%, at least 90%, at least 95%, at least 96%, at least 97%, at least 98%, or at least 99% identical to SEQ ID NO: 9 or SEQ ID NO: 12.

In specific examples, the amino acid sequence of the VH domain includes or consists of SEQ ID NO: 7 and the amino acid sequence of the VL domain includes or consists of SEQ ID NO: 8. In other examples, the amino acid sequence of the VH domain includes or consists of SEQ ID NO: 10 and the amino acid sequence of the VL domain includes or consists of SEQ ID NO: 11.

In particular non-limiting examples, the amino acid sequence of the scFv of the first antigen-binding polypeptide includes or consists of SEQ ID NO: 9 or SEQ ID NO: 12.

In some aspects in which the AbTCR+CSR is targeted to GPC3, the first antigen-binding polypeptide includes a scFv that includes a VH domain and a VL domain, and the VH domain includes the CDR1, CDR2 and CDR3 sequences of the VH domain of GPC3-specific antibody hYP7 (SEQ ID NO: 13), and the VL domain includes the CDR1, CDR2 and CDR3 sequences of the hYP7 VL domain (SEQ ID NO: 14); the second antigen-binding polypeptide includes a VH domain and a heavy chain constant region, wherein the VH domain includes the CDR1, CDR2 and CDR3 sequences of the hYP7 VH domain (SEQ ID NO: 13); and the third antigen-binding polypeptide includes a VL domain and a light chain constant region, wherein the VL domain includes the CDR1, CDR2 and CDR3 sequences of the hYP7 VL domain (SEQ ID NO: 14).

In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14.

In some examples, the amino acid sequence of the VH domain of the first antigen-binding polypeptide is at least 85%, at least 90%, at least 95%, at least 96%, at least 97%, at least 98%, or at least 99% identical to SEQ ID NO: 13 and the amino acid sequence of the VL domain of the first-antigen-binding polypeptide is at least 85%, at least 90%, at least 95%, at least 96%, at least 97%, at least 98%, or at least 99% identical to SEQ ID NO: 14; the amino acid sequence of the VH domain of the second antigen-binding polypeptide is at least 85%, at least 90%, at least 95%, at least 96%, at least 97%, at least 98%, or at least 99% identical to SEQ ID NO: 13; and/or the amino acid sequence of the VL domain of the third antigen-binding polypeptide is at least 85%, at least 90%, at least 95%, at least 96%, at least 97%, at least 98%, or at least 99% identical to SEQ ID NO: 14. In some examples, the amino acid sequence of the scFv of the first antigen-binding polypeptide is at least 85%, at least 90%, at least 95%, at least 96%, at least 97%, at least 98%, or at least 99% identical to SEQ ID NO: 15.

In specific examples, the amino acid sequence of the VH domain of the first antigen-binding polypeptide includes or consists of SEQ ID NO: 13 and the amino acid sequence of the VL domain of the first-antigen-binding polypeptide includes or consists of SEQ ID NO: 14; the amino acid sequence of the VH domain of the second antigen-binding polypeptide includes or consists of SEQ ID NO: 13; and/or the amino acid sequence of the VL domain of the third antigen-binding polypeptide includes or consists of SEQ ID NO: 14.

In particular non-limiting examples, the amino acid sequence of the scFv of the first antigen-binding polypeptide includes or consists of SEQ ID NO: 15.

In other aspects in which the AbTCR+CSR is targeted to GPC3, the first antigen-binding polypeptide includes a single-domain antibody having the CDR1, CDR2 and CDR3 sequences of GPC3-specific antibody HN3 (SEQ ID NO: 16); the second antigen-binding polypeptide includes a VH domain and a heavy chain constant region, wherein the VH domain has the CDR1, CDR2 and CDR3 sequences of the hYP7 VH domain (SEQ ID NO: 13); and the third antigen-binding polypeptide includes a VL domain and a light chain constant region, wherein the VL domain has the CDR1, CDR2 and CDR3 sequences of the hYP7 VL domain (SEQ ID NO: 14).

In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16. In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14.

In some examples, the amino acid sequence of the single-domain antibody of the first antigen-binding polypeptide is at least 85%, at least 90%, at least 95%, at least 96%, at least 97%, at least 98%, or at least 99% identical to SEQ ID NO: 16; the amino acid sequence of the VH domain of the second antigen-binding polypeptide is at least 85%, at least 90%, at least 95%, at least 96%, at least 97%, at least 98%, or at least 99% identical to SEQ ID NO: 13; and/or the amino acid sequence of the VL domain of the third antigen-binding polypeptide is at least 85%, at least 90%, at least 95%, at least 96%, at least 97%, at least 98%, or at least 99% identical to SEQ ID NO: 14.

In specific examples, the amino acid sequence of the single-domain antibody of the first antigen-binding polypeptide includes or consists of SEQ ID NO: 16; the amino acid sequence of the VH domain of the second antigen-binding polypeptide includes or consists of SEQ ID NO: 13; and/or the amino acid sequence of the VL domain of the third antigen-binding polypeptide includes or consists of SEQ ID NO: 14.

In other aspects in which the AbTCR+CSR is targeted to GPC3, the first antigen-binding polypeptide includes a scFv that includes a VH domain and a VL domain, and the VH domain has the CDR1, CDR2 and CDR3 sequences of the hYP7 VH domain (SEQ ID NO: 13) and the VL domain has the CDR1, CDR2 and CDR3 sequences of the hYP7 VL domain (SEQ ID NO: 14); the second antigen-binding polypeptide includes a single-domain antibody and a heavy chain constant region, wherein the single-domain antibody includes the CDR1, CDR2 and CDR3 sequences of HN3 (SEQ ID NO: 16); and the third antigen-binding polypeptide includes a single-domain antibody and a light chain constant region, wherein the single-domain antibody includes the CDR1, CDR2 and CDR3 sequences of HN3 (SEQ ID NO: 16).

In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14. In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16.

In some examples, the amino acid sequence of the VH domain of the first antigen-binding polypeptide is at least 85%, at least 90%, at least 95%, at least 96%, at least 97%, at least 98%, or at least 99% identical to SEQ ID NO: 13; and the amino acid sequence of the VL domain of the first antigen-binding polypeptide is at least 85%, at least 90%, at least 95%, at least 96%, at least 97%, at least 98%, or at least 99% identical to SEQ ID NO: 14; the amino acid sequence of the single-domain antibody of the second antigen-binding polypeptide is at least 85%, at least 90%, at least 95%, at least 96%, at least 97%, at least 98%, or at least 99% identical to SEQ ID NO: 16; and/or the amino acid sequence of the single-domain antibody of the third antigen-binding polypeptide is at least 85%, at least 90%, at least 95%, at least 96%, at least 97%, at least 98%, or at least 99% identical to SEQ ID NO: 16. In some examples, the amino acid sequence of the scFv of the first antigen-binding polypeptide is at least 85%, at least 90%, at least 95%, at least 96%, at least 97%, at least 98%, or at least 99% identical to SEQ ID NO: 15.

In specific examples, the amino acid sequence of the VH domain of the first antigen-binding polypeptide includes or consists of SEQ ID NO: 13; and the amino acid sequence of the VL domain of the first antigen-binding polypeptide includes or consists of SEQ ID NO: 14; the amino acid sequence of the single-domain antibody of the second antigen-binding polypeptide includes or consists of SEQ ID NO: 16; and/or the amino acid sequence of the single-domain antibody of the third antigen-binding polypeptide includes or consists of SEQ ID NO: 16.

In particular non-limiting examples, the amino acid sequence of the scFv of the first antigen-binding polypeptide includes or consists of SEQ ID NO: 15.

In other aspects in which the AbTCR+CSR is targeted to GPC3, the first antigen-binding polypeptide includes a single-domain antibody having the CDR1, CDR2 and CDR3 sequences of HN3 (SEQ ID NO: 16); the second antigen-binding polypeptide includes a single-domain antibody and a heavy chain constant region, wherein the single-domain antibody has the CDR1, CDR2 and CDR3 sequences of HN3 (SEQ ID NO: 16); and the third antigen-binding polypeptide includes a single-domain antibody and a light chain constant region, wherein the single-domain antibody has the CDR1, CDR2 and CDR3 sequences of HN3 (SEQ ID NO: 16).

In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16.

In some examples, the amino acid sequence of the single-domain antibody of the first antigen-binding polypeptide is at least 85%, at least 90%, at least 95%, at least 96%, at least 97%, at least 98%, or at least 99% identical to SEQ ID NO: 16; the amino acid sequence of the single-domain antibody of the second antigen-binding polypeptide is at least 85%, at least 90%, at least 95%, at least 96%, at least 97%, at least 98%, or at least 99% identical to SEQ ID NO: 16; and/or the amino acid sequence of the single-domain antibody of the third antigen-binding polypeptide is at least 85%, at least 90%, at least 95%, at least 96%, at least 97%, at least 98%, or at least 99% identical to SEQ ID NO: 16.

In specific examples, the amino acid sequence of the single-domain antibody of the first antigen-binding polypeptide includes or consists of SEQ ID NO: 16; the amino acid sequence of the single-domain antibody of the second antigen-binding polypeptide includes or consists of SEQ ID NO: 16; and/or the amino acid sequence of the single-domain antibody of the third antigen-binding polypeptide includes or consists of SEQ ID NO: 16.

In some aspects of the disclosed nucleic acid molecules, the amino acid sequence of the heavy chain constant region is at least 85%, at least 90%, at least 95%, at least 96%, at least 97%, at least 98%, or at least 99% identical to SEQ ID NO: 17; and/or the amino acid sequence of the light chain constant region is at least 85%, at least 90%, at least 95%, at least 96%, at least 97%, at least 98%, or at least 99% identical to SEQ ID NO: 18. In some examples, the amino acid sequence of the heavy chain constant region includes or consists of SEQ ID NO: 17; and/or the amino acid sequence of the light chain constant region includes or consists of SEQ ID NO: 18.

In some aspects of the disclosed nucleic acid molecules, the first, second and/or third signal peptide is a mouse IgG kappa signal peptide, such as the mouse IgG kappa signal peptide having the amino acid sequence of SEQ ID NO: 22.

In some aspects in which a single nucleic acid molecule includes the first, second and third modules, each module is separated by a nucleic acid sequence encoding a self-cleaving peptide, such as a 2A peptide.

In some examples, a first self-cleaving peptide sequence is located between the first and second modules and a second self-cleaving peptide sequence is located between the second and third modules. In specific examples, the first self-cleaving peptide and the second self-cleaving peptide are individually selected from P2A and T2A. In particular examples, the first self-cleaving 2A peptide is P2A set forth as SEQ ID NO: 24, and the second self-cleaving 2A peptide is T2A set forth as SEQ ID NO: 25.

In some aspects of the disclosed nucleic acid molecules, the nucleic acid molecule encodes an amino acid sequence at least 85%, at least 90%, at least 95%, at least 96%, at least 97%, at least 98%, or at least 99% identical to SEQ ID NO: 1, SEQ ID NO: 2, SEQ ID NO: 3, SEQ ID NO: 4, SEQ ID NO: 5 or SEQ ID NO: 6. In some examples, the nucleic acid molecule encodes an amino acid sequence that includes or consists of SEQ ID NO: 1, SEQ ID NO: 2, SEQ ID NO: 3, SEQ ID NO: 4, SEQ ID NO: 5 or SEQ ID NO: 6.

In some aspects, the nucleic acid molecule (or each nucleic acid molecule of the set of nucleic acid molecules) is operably linked to a promoter, such as an inducible or constitutive promoter. In some examples, the promoter is a tissue-specific promoter, such as a liver-specific promoter.

In particular non-limiting examples, provided is a nucleic acid molecule encoding an AbTCR and CSR, which includes in the 5′ to 3′ direction: a first module, comprising a nucleic acid sequence encoding a first signal peptide, a nucleic acid sequence encoding a first antigen-binding polypeptide, a nucleic acid sequence encoding a protein tag or linker, and a nucleic acid sequence encoding CD30; a second module, comprising a nucleic acid sequence encoding a second signal peptide, a nucleic acid sequence encoding a second antigen-binding polypeptide, and a nucleic acid sequence encoding a TCR delta chain; and a third module, comprising a nucleic acid sequence encoding a third signal peptide, a nucleic acid sequence encoding a third antigen-binding polypeptide, and a nucleic acid sequence encoding a TCR gamma chain; wherein the first module and the second module are separated by a nucleic acid sequence encoding a self-cleaving 2A peptide, and the second molecule and the third module are separated by a nucleic acid sequence encoding a self-cleaving 2A peptide; and wherein the first, second and third antigen-binding polypeptides specifically bind glypican-2 (GPC2), or the first, second and third antigen-binding polypeptides specifically bind glypican-3 (GPC3). In some instances, the protein tag or linker is a Myc tag; the first, second and/or third signal peptide is a mouse IgG kappa signal peptide; and/or the first self-cleaving 2A peptide and the second self-cleaving 2A peptide are individually selected from P2A and T2A.

Also provided herein are vectors or sets of vectors that include a nucleic acid molecule or set of nucleic acid molecules disclosed herein. Further provided are isolated cells that include a disclosed nucleic acid molecule, set of nucleic acid molecules, vector or set of vectors. In some examples, the cell is an immune cell or an induced pluripotent stem cell (iPSC). In particular examples, the immune cells are T cells (including cytotoxic T cells, T regulatory (Treg) cells, αβT cells, γδT cells, CD3⁺ T cells, CD4⁺ T cells and/or CD8⁺ T cells), NK cells, B cells, mucosal-associated invariant T (MAIT) cells, dendritic cells, macrophages or any other suitable immune cell.

Further provided are compositions that include a pharmaceutically acceptable carrier and an isolated cell disclosed herein.

Iv. Cells Expressing an AbTCR and a CSR

Further provided herein are isolated immune cells or induced pluripotent stem cells (iPSCs) that express an AbTCR and a CSR (AbTCR+CSR) targeted to GPC2 or GPC3. The AbTCR+CSR expressed by the immune cell or iPSC includes a first chimeric polypeptide chain that includes a first antigen-binding polypeptide and a co-stimulatory immune cell signaling domain; a second chimeric polypeptide chain that includes a second antigen-binding polypeptide and a first TCR chain or a transmembrane domain-containing fragment thereof; and a third chimeric polypeptide chain that includes a third antigen-binding polypeptide and a second TCR chain or a transmembrane domain-containing fragment thereof (see FIGS. 1A-1B and SEQ ID NO: 1, SEQ ID NO: 2, SEQ ID NO: 3, SEQ ID NO: 4, SEQ ID NO: 5 and SEQ ID NO: 6 for non-limiting examples of the disclosed AbTCR+CSR). The first, second and third antigen-binding polypeptides specifically bind GPC2, or the first, second and third antigen-binding polypeptides specifically bind GPC3. In some aspects, the first chimeric polypeptide chain further includes a first signal peptide; the second chimeric polypeptide chain further includes a second signal peptide; and/or the third chimeric polypeptide chain further includes a third signal peptide.

In some aspects in which the AbTCR+CSR is targeted to GPC2, the first antigen-binding polypeptide includes a scFv that includes a VH domain and a VL domain, and the VH domain includes the CDR1, CDR2 and CDR3 sequences of the CT3/hCT3-1 VH domain (SEQ ID NO: 7/SEQ ID NO: 10) and the VL domain includes the CDR1, CDR2 and CDR3 sequences of the CT3/hCT3-1 VL domain (SEQ ID NO: 8/SEQ ID NO: 11); the second antigen-binding polypeptide includes a VH domain and a heavy chain constant region, wherein the VH domain includes the CDR1, CDR2 and CDR3 sequences of the CT3/hCT3-1 VH domain (SEQ ID NO: 7/SEQ ID NO: 10); and the third antigen-binding polypeptide includes a VL domain and a light chain constant region, wherein the VL domain includes the CDR1, CDR2 and CDR3 sequences of the CT3/hCT3-1 VL domain (SEQ ID NO: 8/SEQ ID NO: 11).

In some examples, the CT3/hCT3-1 VH domain CDR1, CDR2 and CDR3 sequences respectively include residues 31-35, 50-66 and 99-112 of SEQ ID NO: 7, or residues 26-33, 51-58 and 97-112 of SEQ ID NO: 7. In some examples, the CT3/hCT3-1 VL domain CDR1, CDR2 and CDR3 sequences respectively include residues 24-33, 49-55 and 88-96 of SEQ ID NO: 8, or residues 27-31, 49-51 and 88-96 of SEQ ID NO: 8.

In some examples, the amino acid sequence of the VH domain is at least 85%, at least 90%, at least 95%, at least 96%, at least 97%, at least 98%, or at least 99% identical to SEQ ID NO: 7 and the amino acid sequence of the VL domain is at least 85%, at least 90%, at least 95%, at least 96%, at least 97%, at least 98%, or at least 99% identical to SEQ ID NO: 8. In other examples, the amino acid sequence of the VH domain is at least 85%, at least 90%, at least 95%, at least 96%, at least 97%, at least 98%, or at least 99% identical to SEQ ID NO: 10 and the amino acid sequence of the VL domain is at least 85%, at least 90%, at least 95%, at least 96%, at least 97%, at least 98%, or at least 99% identical to SEQ ID NO: 11. In some examples, the amino acid sequence of the scFv of the first antigen-binding polypeptide is at least 85%, at least 90%, at least 95%, at least 96%, at least 97%, at least 98%, or at least 99% identical to SEQ ID NO: 9 or SEQ ID NO: 12.

In specific examples, the amino acid sequence of the VH domain includes or consists of SEQ ID NO: 7 and the amino acid sequence of the VL domain includes or consists of SEQ ID NO: 8. In other examples, the amino acid sequence of the VH domain includes or consists of SEQ ID NO: 10 and the amino acid sequence of the VL domain includes or consists of SEQ ID NO: 11.

In particular non-limiting examples, the amino acid sequence of the scFv of the first antigen-binding polypeptide includes or consists of SEQ ID NO: 9 or SEQ ID NO: 12.

In some aspects in which the AbTCR+CSR is targeted to GPC3, the first antigen-binding polypeptide includes a scFv that includes a VH domain and a VL domain, and the VH domain includes the CDR1, CDR2 and CDR3 sequences of the hYP7 VH domain (SEQ ID NO: 13) and the VL domain includes the CDR1, CDR2 and CDR3 sequences of the hYP7 VL domain (SEQ ID NO: 14); the second antigen-binding polypeptide includes a VH domain and a heavy chain constant region, wherein the VH domain includes the CDR1, CDR2 and CDR3 sequences of the hYP7 VH domain (SEQ ID NO: 13); and the third antigen-binding polypeptide includes a VL domain and a light chain constant region, wherein the VL domain includes the CDR1, CDR2 and CDR3 sequences of the hYP7 VL domain (SEQ ID NO: 14).

In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14.

In some examples, the amino acid sequence of the VH domain of the first antigen-binding polypeptide is at least 85%, at least 90%, at least 95%, at least 96%, at least 97%, at least 98%, or at least 99% identical to SEQ ID NO: 13 and the amino acid sequence of the VL domain of the first-antigen-binding polypeptide is at least 85%, at least 90%, at least 95%, at least 96%, at least 97%, at least 98%, or at least 99% identical to SEQ ID NO: 14; the amino acid sequence of the VH domain of the second antigen-binding polypeptide is at least 85%, at least 90%, at least 95%, at least 96%, at least 97%, at least 98%, or at least 99% identical to SEQ ID NO: 13; and/or the amino acid sequence of the VL domain of the third antigen-binding polypeptide is at least 85%, at least 90%, at least 95%, at least 96%, at least 97%, at least 98%, or at least 99% identical to SEQ ID NO: 14. In some examples, the amino acid sequence of the scFv of the first antigen-binding polypeptide is at least 85%, at least 90%, at least 95%, at least 96%, at least 97%, at least 98%, or at least 99% identical to SEQ ID NO: 15.

In specific examples, the amino acid sequence of the VH domain of the first antigen-binding polypeptide includes or consists of SEQ ID NO: 13 and the amino acid sequence of the VL domain of the first-antigen-binding polypeptide includes or consists of SEQ ID NO: 14; the amino acid sequence of the VH domain of the second antigen-binding polypeptide includes or consists of SEQ ID NO: 13; and/or the amino acid sequence of the VL domain of the third antigen-binding polypeptide includes or consists of SEQ ID NO: 14.

In particular non-limiting examples, the amino acid sequence of the scFv of the first antigen-binding polypeptide includes or consists of SEQ ID NO: 15.

In other aspects in which the AbTCR+CSR is targeted to GPC3, the first antigen-binding polypeptide includes a single-domain antibody having the CDR1, CDR2 and CDR3 sequences of HN3 (SEQ ID NO: 16); the second antigen-binding polypeptide includes a VH domain and a heavy chain constant region, wherein the VH domain includes the CDR1, CDR2 and CDR3 sequences of the hYP7 VH domain (SEQ ID NO: 13); and the third antigen-binding polypeptide includes a VL domain and a light chain constant region, wherein the VL domain includes the CDR1, CDR2 and CDR3 sequences of the hYP7 VL domain (SEQ ID NO: 14).

In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16. In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14.

In some examples, the amino acid sequence of the single-domain antibody of the first antigen-binding polypeptide is at least 85%, at least 90%, at least 95%, at least 96%, at least 97%, at least 98%, or at least 99% identical to SEQ ID NO: 16; the amino acid sequence of the VH domain of the second antigen-binding polypeptide is at least 85%, at least 90%, at least 95%, at least 96%, at least 97%, at least 98%, or at least 99% identical to SEQ ID NO: 13; and/or the amino acid sequence of the VL domain of the third antigen-binding polypeptide is at least 85%, at least 90%, at least 95%, at least 96%, at least 97%, at least 98%, or at least 99% identical to SEQ ID NO: 14.

In specific examples, the amino acid sequence of the single-domain antibody of the first antigen-binding polypeptide includes or consists of SEQ ID NO: 16; the amino acid sequence of the VH domain of the second antigen-binding polypeptide includes or consists of SEQ ID NO: 13; and/or the amino acid sequence of the VL domain of the third antigen-binding polypeptide includes or consists of SEQ ID NO: 14.

In other aspects in which the AbTCR+CSR is targeted to GPC3, the first antigen-binding polypeptide comprises a scFv having a VH domain and a VL domain, and the VH domain includes the CDR1, CDR2 and CDR3 sequences of the hYP7 VH domain (SEQ ID NO: 13) and the VL domain includes the CDR1, CDR2 and CDR3 sequences of the hYP7 VL domain (SEQ ID NO: 14); the second antigen-binding polypeptide includes a single-domain antibody and a heavy chain constant region, wherein the single-domain antibody has the CDR1, CDR2 and CDR3 sequences of HN3 (SEQ ID NO: 16); and the third antigen-binding polypeptide includes a single-domain antibody and a light chain constant region, wherein the single-domain antibody has the CDR1, CDR2 and CDR3 sequences of HN3 (SEQ ID NO: 16).

In some examples, the hYP7 VH domain CDR1, CDR2 and CDR3 sequences respectively include residues 31-35, 50-68 and 101-106 of SEQ ID NO: 13, or residues 26-33, 51-60 and 99-106 of SEQ ID NO: 13. In some examples, the hYP7 VL domain CDR1, CDR2 and CDR3 sequences respectively include residues 24-40, 56-62 and 95-103 of SEQ ID NO: 14, or residues 27-38, 56-58 and 95-103 of SEQ ID NO: 14. In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16.

In some examples, the amino acid sequence of the VH domain of the first antigen-binding polypeptide is at least 85%, at least 90%, at least 95%, at least 96%, at least 97%, at least 98%, or at least 99% identical to SEQ ID NO: 13; and the amino acid sequence of the VL domain of the first antigen-binding polypeptide is at least 85%, at least 90%, at least 95%, at least 96%, at least 97%, at least 98%, or at least 99% identical to SEQ ID NO: 14; the amino acid sequence of the single-domain antibody of the second antigen-binding polypeptide is at least 85%, at least 90%, at least 95%, at least 96%, at least 97%, at least 98%, or at least 99% identical to SEQ ID NO: 16; and/or the amino acid sequence of the single-domain antibody of the third antigen-binding polypeptide is at least 85%, at least 90%, at least 95%, at least 96%, at least 97%, at least 98%, or at least 99% identical to SEQ ID NO: 16. In some examples, the amino acid sequence of the scFv of the first antigen-binding polypeptide is at least 85%, at least 90%, at least 95%, at least 96%, at least 97%, at least 98%, or at least 99% identical to SEQ ID NO: 15.

In specific examples, the amino acid sequence of the VH domain of the first antigen-binding polypeptide includes or consists of SEQ ID NO: 13; and the amino acid sequence of the VL domain of the first antigen-binding polypeptide includes or consists of SEQ ID NO: 14; the amino acid sequence of the single-domain antibody of the second antigen-binding polypeptide includes or consists of SEQ ID NO: 16; and/or the amino acid sequence of the single-domain antibody of the third antigen-binding polypeptide includes or consists of SEQ ID NO: 16.

In particular non-limiting examples, the amino acid sequence of the scFv of the first antigen-binding polypeptide includes or consists of SEQ ID NO: 15.

In other aspects in which the AbTCR+CSR is targeted to GPC3, the first antigen-binding polypeptide includes a single-domain antibody having the CDR1, CDR2 and CDR3 sequences of HN3; the second antigen-binding polypeptide includes a single-domain antibody and a heavy chain constant region, wherein the single-domain antibody has the CDR1, CDR2 and CDR3 sequences of HN3; and the third antigen-binding polypeptide includes a single-domain antibody and a light chain constant region, wherein the single-domain antibody has the CDR1, CDR2 and CDR3 sequences of HN3.

In some examples, the HN3 CDR1, CDR2 and CDR3 sequences respectively include residues 31-35, 50-65 and 96-105 of SEQ ID NO: 16, or residues 26-33, 51-57 and 96-105 of SEQ ID NO: 16.

In some examples, the amino acid sequence of the single-domain antibody of the first antigen-binding polypeptide is at least 85%, at least 90%, at least 95%, at least 96%, at least 97%, at least 98%, or at least 99% identical to SEQ ID NO: 16; the amino acid sequence of the single-domain antibody of the second antigen-binding polypeptide is at least 85%, at least 90%, at least 95%, at least 96%, at least 97%, at least 98%, or at least 99% identical to SEQ ID NO: 16; and/or the amino acid sequence of the single-domain antibody of the third antigen-binding polypeptide is at least 85%, at least 90%, at least 95%, at least 96%, at least 97%, at least 98%, or at least 99% identical to SEQ ID NO: 16.

In specific examples, the amino acid sequence of the single-domain antibody of the first antigen-binding polypeptide includes or consists of SEQ ID NO: 16; the amino acid sequence of the single-domain antibody of the second antigen-binding polypeptide includes or consists of SEQ ID NO: 16; and/or the amino acid sequence of the single-domain antibody of the third antigen-binding polypeptide includes or consists of SEQ ID NO: 16.

In some aspects of the disclosed immune cells/iPSCs expressing an AbTCR+CSR, the amino acid sequence of the heavy chain constant region is at least 85%, at least 90%, at least 95%, at least 96%, at least 97%, at least 98%, or at least 99% identical to SEQ ID NO: 17 and/or the amino acid sequence of the light chain constant region is at least 85%, at least 90%, at least 95%, at least 96%, at least 97%, at least 98%, or at least 99% identical to SEQ ID NO: 18. In some examples, the amino acid sequence of the heavy chain constant region includes or consists of SEQ ID NO: 17 and/or the amino acid sequence of the light chain constant region includes or consists of SEQ ID NO: 18.

In some aspects, the first chimeric polypeptide chain further includes a protein tag or linker. In some examples, the protein tag is a Myc tag, such as the Myc tag having the amino acid sequence of SEQ ID NO: 23.

In some aspects, the co-stimulatory immune cell signaling domain includes all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

In some aspects, the first TCR chain or the transmembrane domain-containing fragment thereof is derived from a TCR γ chain, and the second TCR chain or the transmembrane domain-containing fragment thereof is derived from a TCR δ chain; or the first TCR chain or the transmembrane domain-containing fragment thereof is derived from a TCR δ chain, and the second TCR chain or the transmembrane domain-containing fragment thereof is derived from a TCR γ chain. In other aspects, the first TCR chain or the transmembrane domain-containing fragment thereof is derived from a TCR α chain, and the second TCR chain or the transmembrane domain-containing fragment thereof is derived from a TCR β chain; or the first TCR chain or the transmembrane domain-containing fragment thereof is derived from a TCR β chain, and the second TCR chain or the transmembrane domain-containing fragment thereof is derived from a TCR α chain.

In some aspects of the disclosed immune cells/iPSCs expressing an AbTCR+CSR, the first, second and/or third signal peptide is a mouse IgG kappa signal peptide, such as the mouse IgG kappa signal peptide having the amino acid sequence of SEQ ID NO: 22.

In some aspects of the disclosed immune cells/iPSCs expressing an AbTCR+CSR, the amino acid sequence of the first chimeric polypeptide chain includes residues 21-556 of SEQ ID NO: 1, the amino acid sequence of the second chimeric polypeptide chain includes residues 604-897 of SEQ ID NO: 1, and the amino acid sequence of the third chimeric polypeptide chain includes residues 944-1223 of SEQ ID NO: 1. In other aspects, the amino acid sequence of the first chimeric polypeptide chain includes residues 21-556 of SEQ ID NO: 2, the amino acid sequence of the second chimeric polypeptide chain includes residues 604-897 of SEQ ID NO: 2, and the amino acid sequence of the third chimeric polypeptide chain includes residues 944-1223 of SEQ ID NO: 2. In other aspects, the amino acid sequence of the first chimeric polypeptide chain includes residues 21-557 of SEQ ID NO: 3, the amino acid sequence of the second chimeric polypeptide chain includes residues 605-892 of SEQ ID NO: 3, and the amino acid sequence of the third chimeric polypeptide chain includes residues 939-1225 of SEQ ID NO: 3. In other aspects, the amino acid sequence of the first chimeric polypeptide chain includes residues 21-421 of SEQ ID NO: 4, the amino acid sequence of the second chimeric polypeptide chain includes residues 469-756 of SEQ ID NO: 4, and the amino acid sequence of the third chimeric polypeptide chain includes residues 803-1089 of SEQ ID NO: 4. In other aspects, the amino acid sequence of the first chimeric polypeptide chain includes residues 21-557 of SEQ ID NO: 5, the amino acid sequence of the second chimeric polypeptide chain includes residues 605-891 of SEQ ID NO: 5, and the amino acid sequence of the third chimeric polypeptide chain includes residues 938-1227 of SEQ ID NO: 5. In other aspects, the amino acid sequence of the first chimeric polypeptide chain includes residues 21-421 of SEQ ID NO: 6, the amino acid sequence of the second chimeric polypeptide chain includes residues 469-755 of SEQ ID NO: 6, and the amino acid sequence of the third chimeric polypeptide chain includes residues 802-1091 of SEQ ID NO: 6.

In particular examples, the isolated immune cell or iPSC expressing an AbTCR and CSR includes a first chimeric polypeptide chain, comprising a first antigen-binding polypeptide, a protein tag or linker, and CD30; a second chimeric polypeptide chain, comprising a second antigen-binding polypeptide, and a TCR delta chain; and a third chimeric polypeptide chain, comprising a third antigen-binding polypeptide, and a TCR gamma chain, wherein the first, second and third antigen-binding polypeptides specifically bind glypican-2 (GPC2), or the first, second and third antigen-binding polypeptides specifically bind glypican-3 (GPC3). In some instances, the first chimeric polypeptide chain further comprises a first signal peptide; the second chimeric polypeptide chain further comprises a second signal peptide; and/or the third chimeric polypeptide chain further comprises a third signal peptide. The first, second and/or third signal peptide is, in some examples, a mouse IgG kappa signal peptide. In some instances, the protein tag or linker is a Myc tag.

In some aspects, the immune cells are T cells (including cytotoxic T cells, T regulatory (Treg) cells, αβT cells, γδT cells, CD3⁺ T cells, CD4⁺ T cells and/or CD8⁺ T cells), NK cells, B cells, MAIT cells, dendritic cells, macrophages or any other suitable immune cell.

Compositions that include a pharmaceutically acceptable carrier and an immune cell or iPSC expressing an AbTCR and are also provided (see section VII below).

Further provided are methods of treating a GPC2-positive or GPC3-positive cancer in a subject, or inhibiting tumor growth and/or metastasis of a GPC2-positive or GPC3-positive cancer in a subject, by administering to the subject a therapeutically effective amount of an isolated immune cell or iPSC expressing an AbTCR, or composition thereof, as disclosed herein (see section VIII below). In some aspects, the GPC2-positive cancer is neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma, small cell lung cancer, or osteosarcoma. In some aspects, the GPC3-positive cancer is a hepatocellular carcinoma (HCC), melanoma, ovarian clear-cell carcinoma, yolk sac tumor (YST), hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma, salivary gland cancer, glioblastoma, choriocarcinoma, rhabdosarcoma, renal clear-cell carcinoma, or embryonal tumor.

### V. Anti-GPC2 and Anti-GPC3 Antibody Sequences

The AbTCRs and CSRs disclosed herein include an antibody or antigen-binding fragment (such as scFv, VH domain, VL domain or single-domain antibody) that specifically binds GPC2 or GPC3. Such antibodies or antigen-binding fragments are referred to as anti-GPC2, anti-GPC3, GPC2-specific, GPC3-specific, GPC2-targeted, or GPC3-targeted antibodies or antigen-binding fragments, and the AbTCRs, CSRs, or CARs comprising such antibodies or antigen-binding fragments are referred to with the corresponding antigen specificity terminology.

In some aspects, the GPC2-specific antibody is CT3, a murine monoclonal antibody, or a humanized version thereof (hCT3-1). The amino acid sequences of CT3 and hCT3-1 are provided below. Tables 1 and 2 list the amino acid positions of the CDR1, CDR2 and CDR3 of the VH domain and VL domain, respectively, as determined using Kabat and IMGT. The CDR boundaries can also be defined using an alternative numbering scheme, such as the Chothia or Paratome numbering scheme.

In some aspects, the GPC3-specific antibody is hYP7, a humanized mouse antibody, or HN3, a human single-domain (VH) monoclonal antibody. The amino acid sequences of hYP7 and HN3 are provided below. Tables 3-5 list the amino acid positions of the CDR1, CDR2 and CDR3 of the VH and VL domains of hYP7, and the CDR1, CDR2 and CDR3 of HN3, as determined using Kabat and IMGT. The CDR boundaries can also be defined using an alternative numbering scheme, such as the Chothia or Paratome numbering scheme.

The scFv (VL-linker-VH orientation) amino acid sequences of the CT3, hCT3 and hYP7 antibodies are also listed below. In each scFv sequence, the VH and VL domains are separated by a linker, which is underlined.

CT3 V_{H} nucleic acid sequence
(SEQ ID NO: 26)
GAGGTCCAGCTGCAACAGTCTGGACCTGAACTGGTGAAGCCTGGG
GCTTCAGTAAAGATGTCCTGCAAGGCTTCTAGATTCACATTCACT
GACTACAACATACACTGGGTGAAGCAGAGCCCTGGAAAGACCCTT
GAATGGATTGGATATATTAACCCTAACAATGGTGATATTTTCTAC
AAACAGAAGTTCAATGGCAAGGCCACATTGACTATAAACAAGTCC
TCCAACACAGCCTACATGGAGCTCCGCAGCCTGACATCGGAGGAT
TCTGCAGTCTATTACTGTGTAAGATCCTCTAATATTCGTTATACT
TTCGACAGGTTCTTCGATGTCTGGGGCACAGGGACCACGGTCACC
GTCTCCTCA
CT3 V_{H} amino acid sequence
(SEQ ID NO: 7)
EVQLQQSGPELVKPGASVKMSCKASRFTFTDYNIHWVKQSPGKTL
EWIGYINPNNGDIFYKQKENGKATLTINKSSNTAYMELRSLTSED
SAVYYCVRSSNIRYTFDRFFDVWGTGTTVTVSS

TABLE 1
Locations of the CDRs in the CT3/hCT3
VH domain (SEQ ID NO: 7/SEQ ID NO: 10)

| Scheme | CDR1 | CDR2 | CDR3 |
|---|---|---|---|
| Kabat | 31-35 | 50-66 | 99-112 |
| IMGT | 26-33 | 51-58 | 97-112 |

CT3 V_{L} nucleic acid sequence
(SEQ ID NO: 27)
GAAAATGTGCTCACCCAGTCTCCAGCAATCATGTCTGCATCTCTA
GGGGAGAAGGTCACCATGAGCTGCAGGGCCAGCTCAAGTGTAAAT
TACATTTACTGGTACCAGCAGAAGTCAGATGCCTCCCCCAAACTA
TGGATTTATTACACATCCAACCTGGCTCCTGGAGTCCCAGCTCGC
TTCAGTGGCAGTGGGTCTGGGAACTCTTATTCTCTCACAATCAGC
AGCATGGAGGGTGAAGATGCTGCCACTTATTACTGCCAGCAGTTT
TCTAGTTCCCCATCCACGTTCGGTACTGGGACCAAGCTGGAGCTG
AAA
CT3 V_{L} amino acid sequence
(SEQ ID NO: 8)
ENVLTQSPAIMSASLGEKVTMSCRASSSVNYIYWYQQKSDASPKL
WIYYTSNLAPGVPARFSGSGSGNSYSLTISSMEGEDAATYYCQQF
SSSPSTFGTGTKLELK

TABLE 2
Locations of the CDRs in the CT3/hCT3
VL domain (SEQ ID NO: 8/SEQ ID NO: 11)

| Scheme | CDR1 | CDR2 | CDR3 |
|---|---|---|---|
| Kabat | 24-33 | 49-55 | 88-96 |
| IMGT | 27-31 | 49-51 | 88-96 |

CT3 (VL-linker-VH) scFv
(SEQ ID NO: 9)
ENVLTQSPAIMSASLGEKVTMSCRASSSVNYIYWYQQKSDASPKL
WIYYTSNLAPGVPARFSGSGSGNSYSLTISSMEGEDAATYYCQQF
SSSPSTFGTGTKLELKRSRGGGGSGGGGSGGGGSLEMAEVQLQQS
GPELVKPGASVKMSCKASRFTFTDYNIHWVKQSPGKTLEWIGYIN
PNNGDIFYKQKENGKATLTINKSSNTAYMELRSLTSEDSAVYYCV
RSSNIRYTFDRFFDVWGTGTTVTVSS
hCT3-1 VH nucleic acid sequence
(SEQ ID NO: 28)
CAAGTACAGCTTGTACAATCCGGAGCGGAAGTAAAGAAGCCCGGG
GCCTCCGTGAAAGTCAGCTGCAAAGCGTCTAGATTCACCTTCACT
GACTATAACATCCACTGGGTGCGGCAAGCGCCTGGTCAGGGCCTC
GAATGGATTGGCTATATAAACCCGAACAACGGGGACATTTTCTAT
AAGCAGAAATTCAATGGCAGAGTGACGCTCACCGCGGACAAAAGT
ACCAGTACTGCTTATATGGAACTGTCTAGTCTTACGAGTGAGGAT
ACCGCTGTGTATTATTGCGTGAGGTCCTCCAATATACGCTATACG
TTTGATAGATTCTTTGATGTATGGGGACAGGGAACCCTCGTCACG
GTCAGCTCA
hCT3-1 VH amino acid sequence
(SEQ ID NO: 10)
QVQLVQSGAEVKKPGASVKVSCKASRFTFTDYNIHWVRQAPGQGL
EWIGYINPNNGDIFYKQKFNGRVTLTADKSTSTAYMELSSLTSED
TAVYYCVRSSNIRYTFDRFFDVWGQGTLVTVSS
hCT3-1 VL nucleic acid sequence
(SEQ ID NO: 29)
GACGTAGTAATGACTCAAAGCCCCCTCTCCTTGCCAGTGACGCCA
GGTGAGCCGGCATCTATTTCTTGCAGGGCTAGTTCTTCAGTGAAC
TACATCTATTGGTATTTGCAGAAGCCAGGACAAAGTCCGCAGCTT
TGGATATATTACACGAGCAACCTTGCGCCAGGTGTTCCAGATCGC
TTCTCTGGCAGTGGATCAGGGACGGACTTCACACTGAAGATCTCT
CGCGTTGAAGCTGAAGACGTCGGCGTGTACTATTGCCAACAATTC
AGTAGTTCTCCGAGCACTTTTGGCCAGGGAACCAAGCTTGAGATT
AAA
hCT3-1 VL amino acid sequence
(SEQ ID NO: 11)
DVVMTQSPLSLPVTPGEPASISCRASSSVNYIYWYLQKPGQSPQL
WIYYTSNLAPGVPDRFSGSGSGTDFTLKISRVEAEDVGVYYCQQF
SSSPSTFGQGTKLEIK
hCT3-1 (VL-linker-VH) scFv
(SEQ ID NO: 12)
DVVMTQSPLSLPVTPGEPASISCRASSSVNYIYWYLQKPGQSPQL
WIYYTSNLAPGVPDRFSGSGSGTDFTLKISRVEAEDVGVYYCQQF
SSSPSTFGQGTKLEIKRSRGGGGSGGGGSGGGGSLEMAQVQLVQS
GAEVKKPGASVKVSCKASRFTFTDYNIHWVRQAPGQGLEWIGYIN
PNNGDIFYKQKFNGRVTLTADKSTSTAYMELSSLTSEDTAVYYCV
RSSNIRYTFDRFFDVWGQGTLVTVSS
hYP7 VH nucleic acid sequence
(SEQ ID NO: 30)
GAGGTGCAGCTTGTTGAGTCTGGTGGAGGATTGGTGCAGCCTGGA
GGGTCATTGAGACTCTCATGTGCAGCCTCTGGATTCACCTTCAAT
AAGAATGCCATGAATTGGGTCCGCCAGGCTCCAGGAAAGGGTTTG
GAATGGGTTGGCCGCATAAGAAATAAAACTAATAATTATGCAACA
TATTATGCCGATTCAGTGAAAGCCAGGTTTACCATCTCCAGAGAT
GATTCAAAGAACTCACTCTATCTGCAAATGAACAGCTTGAAAACC
GAGGACACAGCCGTGTACTATTGTGTGGCTGGTAACTCGTTTGCT
TACTGGGGCCAAGGGACTCTGGTCACTGTCTCTGCA
hYP7 VH Protein Sequence
(SEQ ID NO: 13)
EVQLVESGGGLVQPGGSLRLSCAASGFTFNKNAMNWVRQAPGKGL
EWVGRIRNKTNNYATYYADSVKARFTISRDDSKNSLYLQMNSLKT
EDTAVYYCVAGNSFAYWGQGTLVTVSA

TABLE 3
Locations of the CDRs in the hYP7 VH domain (SEQ ID NO: 13)

| Scheme | CDR1 | CDR2 | CDR3 |
|---|---|---|---|
| Kabat | 31-35 | 50-68 | 101-106 |
| IMGT | 26-33 | 51-60 | 99-106 |

hYP7 VL nucleic acid sequence
(SEQ ID NO: 31)
GACATTGTGATGACCCAGTCTCCAGACTCCCTAGCTGTGTCACTG
GGAGAGAGGGCCACTATCAACTGCAAGTCCAGTCAGAGCCTTTTA
TATAGCAGCAATCAAAAGAACTACTTGGCCTGGTACCAACAGAAA
CCAGGGCAGCCTCCTAAACTGCTGATTTACTGGGCATCCAGTAGG
GAATCTGGGGTCCCTGATCGCTTCAGTGGCAGTGGATCTGGGACA
GATTTCACTCTCACCATCAGCAGTCTGCAGGCTGAAGACGTGGCA
GTTTATTACTGTCAGCAATATTATAACTATCCGCTCACGTTCGGT
CAGGGGACCAAGTTGGAGATCAAA
hYP7 VL Protein Sequence
(SEQ ID NO: 14)
DIVMTQSPDSLAVSLGERATINCKSSQSLLYSSNQKNYLAWYQQK
PGQPPKLLIYWASSRESGVPDRFSGSGSGTDFTLTISSLQAEDVA
VYYCQQYYNYPLTFGQGTKLEIK

TABLE 4
Locations of the CDRs in the hYP7 VL domain (SEQ ID NO: 14)

| Scheme | CDR1 | CDR2 | CDR3 |
|---|---|---|---|
| Kabat | 24-40 | 56-62 | 95-103 |
| IMGT | 27-38 | 56-58 | 95-103 |

hYP7 (VL-linker-VH) scFv
(SEQ ID NO: 15)
DIVMTQSPDSLAVSLGERATINCKSSQSLLYSSNQKNYLAWYQQK
PGQPPKLLIYWASSRESGVPDRFSGSGSGTDFTLTISSLQAEDVA
VYYCQQYYNYPLTFGQGTKLEIKRSRGGGGSGGGGSGGGGSLEMA
EVQLVESGGGLVQPGGSLRLSCAASGFTFNKNAMNWVRQAPGKGL
EWVGRIRNKTNNYATYYADSVKARFTISRDDSKNSLYLQMNSLKT
EDTAVYYCVAGNSFAYWGQGTLVTVSA
HN3 nucleic acid sequence
(SEQ ID NO: 32)
CAGGTGCAGCTGGTGCAGTCTGGGGGAGGCTTGGTACAGCCTGGA
GGGTCCCTGAGACTCTCCTGTGCAGCCTCTTATTTCGATTTCGAT
TCTTATGAAATGAGCTGGGTCCGCCAGGCTCCAGGGAAGGGCCTA
GAGTGGATTGGGAGTATCTATCATAGTGGGAGCACCTACTACAAC
CCGTCCCTCAAGAGTCGAGTCACCATCTCCAGAGACAATTCCAAG
AACACGCTGTATCTGCAAATGAACACCCTGAGAGCCGAGGACACA
GCCACGTATTACTGTGCGAGAGTAAATATGGACCGATTTGACTAC
TGGGGCCAGGGAACCCTGGTCACCGTCTCCTCAAGT
HN3 Protein Sequence
(SEQ ID NO: 16)
QVQLVQSGGGLVQPGGSLRLSCAASYFDFDSYEMSWVRQAPGKGLE
WIGSIYHSGSTYYNPSLKSRVTISRDNSKNTLYLQMNTLRAEDTAT
YYCARVNMDRFDYWGQGTLVTVSS

TABLE 5
Locations of the CDRs in HN3 (SEQ ID NO: 16)

| Scheme | CDR1 | CDR2 | CDR3 |
|---|---|---|---|
| Kabat | 31-35 | 50-65 | 96-105 |
| IMGT | 26-33 | 51-57 | 96-105 |

### VI. AbTCR and CSR Sequences

GPC2- and GPC3-targeted AbTCR+CSRs were generated using the CT3, hCT3-1, hYP7 and HN3 antibodies. Six exemplary AbTCR+CSR amino acid sequences (two GPC2-specific and four GPC-specific) are provided below. Schematics of the six exemplary AbTCR+CSRs are shown in FIGS. 1A-1B. The amino acid sequences of the individual components of the AbTCR+CSRs, as well as exemplary nucleic acid sequences encoding individual components of the AbTCR+CSRs, are also provided.

### A. Amino Acid Sequences

ART-CT3-CT3 AbTCR + CSR
(SEQ ID NO: 1)
METDTLLLWVLLLWVPGSTGENVLTQSPAIMSASLGEKVTMSCRA
SSSVNYIYWYQQKSDASPKLWIYYTSNLAPGVPARFSGSGSGNSY
SLTISSMEGEDAATYYCQQFSSSPSTFGTGTKLELKRSRGGGGSG
GGGSGGGGSLEMAEVQLQQSGPELVKPGASVKMSCKASRFTFTDY
NIHWVKQSPGKTLEWIGYINPNNGDIFYKQKFNGKATLTINKSSN
TAYMELRSLTSEDSAVYYCVRSSNIRYTFDRFFDVWGTGTTVTVS
SEQKLISEEDLAAATGAPPLGTQPDCNPTPENGEAPASTSPTQSL
LVDSQASKTLPIPTSAPVALSSTGKPVLDAGPVLFWVILVLVVVV
GSSAFLLCHRRACRKRIRQKLHLCYPVQTSQPKLELVDSRPRRSS
TQLRSGASVTEPVAEERGLMSQPLMETCHSVGAAYLESLPLQDAS
PAGGPSSPRDLPEPRVSTEHTNNKIEKIYIMKADTVIVGTVKAEL
PEGRGLAGPAEPELEEELEADHTPHYPEQETEPPLGSCSDVMLSV
EEEGKEDPLPTAASGKRAKRSGSGATNFSLLKQAGDVEENPGPME
TDTLLLWVLLLWVPGSTGEVQLQQSGPELVKPGASVKMSCKASRF
TFTDYNIHWVKQSPGKTLEWIGYINPNNGDIFYKQKFNGKATLTI
NKSSNTAYMELRSLTSEDSAVYYCVRSSNIRYTFDRFFDVWGTGT
TVTVSSASTKGPSVFPLAPSSKSTSGGTAALGCLVKDYFPEPVTV
SWNSGALTSGVHTFPAVLQSSGLYSLSSVVTVPSSSLGTQTYICN
VNHKPSNTKVDKRVEPKSCEVKTDSTDHVKPKETENTKQPSKSCH
KPKAIVHTEKVNMMSLTVLGLRMLFAKTVAVNFLLTAKLFFLRAK
RSGSGEGRGSLLTCGDVEENPGPMETDTLLLWVLLLWVPGSTGEN
VLTQSPAIMSASLGEKVTMSCRASSSVNYIYWYQQKSDASPKLWI
YYTSNLAPGVPARFSGSGSGNSYSLTISSMEGEDAATYYCQQFSS
SPSTFGTGTKLELKRTVAAPSVFIFPPSDEQLKSGTASVVCLLNN
FYPREAKVQWKVDNALQSGNSQESVTEQDSKDSTYSLSSTLTLSK
ADYEKHKVYACEVTHQGLSSPVTKSFNRGECPIKTDVITMDPKDN
CSKDANDTLLLQLTNTSAYYMYLLLLLKSVVYFAITCCLLRRTAF
CCNGEKS

| Residues | Feature |
|---|---|
| 1-20 | Mouse IgG kappa signal peptide |
| 21-126 | CT3 VL domain |
| 149-271 | CT3 VH domain |
| 21-271 | CT3 (VL-VH) scFv |
| 272-281 | c-myc tag |
| 282-556 | CD30 (TNFRSF8) fragment |
| 562-583 | P2A peptide |
| 584-603 | Mouse IgG kappa signal peptide |
| 604-726 | CT3 VH domain |
| 727-833 | Human IgG1 CH1 domain |
| 834-897 | TCR delta chain fragment |
| 903-923 | T2A peptide |
| 924-943 | Mouse IgG kappa signal peptide |
| 944-1049 | CT3 VL domain |
| 1050-1156 | IgG kappa constant region |
| 1157-1223 | TCR gamma chain fragment |

ART-hCT3-1-hCT3-1 AbTCR + CSR
(SEQ ID NO: 2)
METDTLLLWVLLLWVPGSTGDVVMTQSPLSLPVTPGEPASISCRA
SSSVNYIYWYLQKPGQSPQLWIYYTSNLAPGVPDRFSGSGSGTDF
TLKISRVEAEDVGVYYCQQFSSSPSTFGQGTKLEIKRSRGGGGSG
GGGSGGGGSLEMAQVQLVQSGAEVKKPGASVKVSCKASRFTFTDY
NIHWVRQAPGQGLEWIGYINPNNGDIFYKQKFNGRVTLTADKSTS
TAYMELSSLTSEDTAVYYCVRSSNIRYTFDRFFDVWGQGTLVTVS
SEQKLISEEDLAAATGAPPLGTQPDCNPTPENGEAPASTSPTQSL
LVDSQASKTLPIPTSAPVALSSTGKPVLDAGPVLFWVILVLVVVV
GSSAFLLCHRRACRKRIRQKLHLCYPVQTSQPKLELVDSRPRRSS
TQLRSGASVTEPVAEERGLMSQPLMETCHSVGAAYLESLPLQDAS
PAGGPSSPRDLPEPRVSTEHTNNKIEKIYIMKADTVIVGTVKAEL
PEGRGLAGPAEPELEEELEADHTPHYPEQETEPPLGSCSDVMLSV
EEEGKEDPLPTAASGKRAKRSGSGATNFSLLKQAGDVEENPGPME
TDTLLLWVLLLWVPGSTGQVQLVQSGAEVKKPGASVKVSCKASRF
TFTDYNIHWVRQAPGQGLEWIGYINPNNGDIFYKQKFNGRVTLTA
DKSTSTAYMELSSLTSEDTAVYYCVRSSNIRYTFDRFFDVWGQGT
LVTVSSASTKGPSVFPLAPSSKSTSGGTAALGCLVKDYFPEPVTV
SWNSGALTSGVHTFPAVLQSSGLYSLSSVVTVPSSSLGTQTYICN
VNHKPSNTKVDKRVEPKSCEVKTDSTDHVKPKETENTKQPSKSCH
KPKAIVHTEKVNMMSLTVLGLRMLFAKTVAVNFLLTAKLFFLRAK
RSGSGEGRGSLLTCGDVEENPGPMETDTLLLWVLLLWVPGSTGDV
VMTQSPLSLPVTPGEPASISCRASSSVNYIYWYLQKPGQSPQLWI
YYTSNLAPGVPDRFSGSGSGTDFTLKISRVEAEDVGVYYCQQFSS
SPSTFGQGTKLEIKRTVAAPSVFIFPPSDEQLKSGTASVVCLLNN
FYPREAKVQWKVDNALQSGNSQESVTEQDSKDSTYSLSSTLTLSK
ADYEKHKVYACEVTHQGLSSPVTKSFNRGECPIKTDVITMDPKDN
CSKDANDTLLLQLTNTSAYYMYLLLLLKSVVYFAIITCCLLRRTA
FCCNGEKS

| Residues | Feature |
|---|---|
| 1-20 | Mouse IgG kappa signal peptide |
| 21-126 | hCT3 VL domain |
| 149-271 | hCT3 VH domain |
| 21-271 | hCT3 (VL-VH) scFv |
| 272-281 | c-myc tag |
| 282-556 | CD30 (TNFRSF8) fragment |
| 562-583 | P2A peptide |
| 584-603 | Mouse IgG kappa signal peptide |
| 604-726 | hCT3 VH domain |
| 727-833 | Human IgG1 CH1 domain |
| 834-897 | TCR delta chain fragment |
| 903-923 | T2A peptide |
| 924-943 | Mouse IgG kappa signal peptide |
| 944-1049 | hCT3 VL domain |
| 1050-1156 | IgG kappa constant region |
| 1157-1223 | TCR gamma chain fragment |

ART-hYP7-hYP7 AbTCR + CSR
(SEQ ID NO: 3)
METDTLLLWVLLLWVPGSTGDIVMTQSPDSLAVSLGERATINCKS
SQSLLYSSNQKNYLAWYQQKPGQPPKLLIYWASSRESGVPDRFSG
SGSGTDFTLTISSLQAEDVAVYYCQQYYNYPLTFGQGTKLEIKRS
RGGGGSGGGGSGGGGSLEMAEVQLVESGGGLVQPGGSLRLSCAAS
GFTFNKNAMNWVRQAPGKGLEWVGRIRNKTNNYATYYADSVKARF
TISRDDSKNSLYLQMNSLKTEDTAVYYCVAGNSFAYWGQGTLVTV
SAEQKLISEEDLAAATGAPPLGTQPDCNPTPENGEAPASTSPTQS
LLVDSQASKTLPIPTSAPVALSSTGKPVLDAGPVLFWVILVLVVV
VGSSAFLLCHRRACRKRIRQKLHLCYPVQTSQPKLELVDSRPRRS
STQLRSGASVTEPVAEERGLMSQPLMETCHSVGAAYLESLPLQDA
SPAGGPSSPRDLPEPRVSTEHTNNKIEKIYIMKADTVIVGTVKAE
LPEGRGLAGPAEPELEEELEADHTPHYPEQETEPPLGSCSDVMLS
VEEEGKEDPLPTAASGKRAKRSGSGATNFSLLKQAGDVEENPGPM
ETDTLLLWVLLLWVPGSTGEVQLVESGGGLVQPGGSLRLSCAASG
FTFNKNAMNWVRQAPGKGLEWVGRIRNKTNNYATYYADSVKARFT
ISRDDSKNSLYLQMNSLKTEDTAVYYCVAGNSFAYWGQGTLVTVS
AASTKGPSVFPLAPSSKSTSGGTAALGCLVKDYFPEPVTVSWNSG
ALTSGVHTFPAVLQSSGLYSLSSVVTVPSSSLGTQTYICNVNHKP
SNTKVDKRVEPKSCEVKTDSTDHVKPKETENTKQPSKSCHKPKAI
VHTEKVNMMSLTVLGLRMLFAKTVAVNFLLTAKLFFLRAKRSGSG
EGRGSLLTCGDVEENPGPMETDTLLLWVLLLWVPGSTGDIVMTQS
PDSLAVSLGERATINCKSSQSLLYSSNQKNYLAWYQQKPGQPPKL
LIYWASSRESGVPDRFSGSGSGTDFTLTISSLQAEDVAVYYCQQY
YNYPLTFGQGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGECPIKTDVITMDPK
DNCSKDANDTLLLQLTNTSAYYMYLLLLLKSVVYFAIITCCLLRR
TAFCCNGEKS

| Residues | Feature |
|---|---|
| 1-20 | Mouse IgG kappa signal peptide |
| 21-133 | hYP7 VL domain |
| 156-272 | hYP7 VH domain |
| 21-272 | hYP7 (VL-VH) scFv |
| 273-282 | c-myc tag |
| 283-557 | CD30 (TNFRSF8) fragment |
| 563-584 | P2A peptide |
| 585-604 | Mouse IgG kappa signal peptide |
| 605-721 | hYP7 VH domain |
| 722-828 | Human IgG1 CH1 domain |
| 829-892 | TCR delta chain fragment |
| 898-918 | T2A peptide |
| 919-938 | Mouse IgG kappa signal peptide |
| 939-1051 | hYP7 VL domain |
| 1052-1158 | IgG kappa constant region |
| 1159-1225 | TCR gamma chain fragment |

ART-hYP7-HN3 AbTCR + CSR
(SEQ ID NO: 4)
METDTLLLWVLLLWVPGSTGQVQLVQSGGGLVQPGGSLRLSCAAS
YFDFDSYEMSWVRQAPGKGLEWIGSIYHSGSTYYNPSLKSRVTIS
RDNSKNTLYLQMNTLRAEDTATYYCARVNMDRFDYWGQGTLVTVS
SEQKLISEEDLAAATGAPPLGTQPDCNPTPENGEAPASTSPTQSL
LVDSQASKTLPIPTSAPVALSSTGKPVLDAGPVLFWVILVLVVVV
GSSAFLLCHRRACRKRIRQKLHLCYPVQTSQPKLELVDSRPRRSS
TQLRSGASVTEPVAEERGLMSQPLMETCHSVGAAYLESLPLQDAS
PAGGPSSPRDLPEPRVSTEHTNNKIEKIYIMKADTVIVGTVKAEL
PEGRGLAGPAEPELEEELEADHTPHYPEQETEPPLGSCSDVMLSV
EEEGKEDPLPTAASGKRAKRSGSGATNFSLLKQAGDVEENPGPME
TDTLLLWVLLLWVPGSTGEVQLVESGGGLVQPGGSLRLSCAASGF
TFNKNAMNWVRQAPGKGLEWVGRIRNKTNNYATYYADSVKARFTI
SRDDSKNSLYLQMNSLKTEDTAVYYCVAGNSFAYWGQGTLVTVSA
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCEVKTDSTDHVKPKETENTKQPSKSCHKPKAIV
HTEKVNMMSLTVLGLRMLFAKTVAVNFLLTAKLFFLRAKRSGSGE
GRGSLLTCGDVEENPGPMETDTLLLWVLLLWVPGSTGDIVMTQSP
DSLAVSLGERATINCKSSQSLLYSSNQKNYLAWYQQKPGQPPKLL
IYWASSRESGVPDRFSGSGSGTDFTLTISSLQAEDVAVYYCQQYY
NYPLTFGQGTKLEIKRTVAAPSVFIFPPSDEQLKSGTASVVCLLN
NFYPREAKVQWKVDNALQSGNSQESVTEQDSKDSTYSLSSTLTLS
KADYEKHKVYACEVTHQGLSSPVTKSFNRGECPIKTDVITMDPKD
NCSKDANDTLLLQLTNTSAYYMYLLLLLKSVVYFAIITCCLLRRT
AFCCNGEKS

| Residues | Feature |
|---|---|
| 1-20 | Mouse IgG kappa signal peptide |
| 21-136 | HN3 (VHH) |
| 137-146 | c-myc tag |
| 147-421 | CD30 (TNFRSF8) fragment |
| 427-448 | P2A peptide |
| 449-468 | Mouse IgG kappa signal peptide |
| 469-585 | hYP7 VH domain |
| 586-692 | Human IgG1 CH1 domain |
| 693-756 | TCR delta chain fragment |
| 762-782 | T2A peptide |
| 783-802 | Mouse IgG kappa signal peptide |
| 803-915 | hYP7 VL domain |
| 916-1022 | IgG kappa constant region |
| 1023-1089 | TCR gamma chain fragment |

ART-HN3-hYP7 AbTCR + CSR
(SEQ ID NO: 5)
METDTLLLWVLLLWVPGSTGDIVMTQSPDSLAVSLGERATINCKS
SQSLLYSSNQKNYLAWYQQKPGQPPKLLIYWASSRESGVPDRFSG
SGSGTDFTLTISSLQAEDVAVYYCQQYYNYPLTFGQGTKLEIKRS
RGGGGSGGGGSGGGGSLEMAEVQLVESGGGLVQPGGSLRLSCAAS
GFTFNKNAMNWVRQAPGKGLEWVGRIRNKTNNYATYYADSVKARF
TISRDDSKNSLYLQMNSLKTEDTAVYYCVAGNSFAYWGQGTLVTV
SAEQKLISEEDLAAATGAPPLGTQPDCNPTPENGEAPASTSPTQS
LLVDSQASKTLPIPTSAPVALSSTGKPVLDAGPVLFWVILVLVVV
VGSSAFLLCHRRACRKRIRQKLHLCYPVQTSQPKLELVDSRPRRS
STQLRSGASVTEPVAEERGLMSQPLMETCHSVGAAYLESLPLQDA
SPAGGPSSPRDLPEPRVSTEHTNNKIEKIYIMKADTVIVGTVKAE
LPEGRGLAGPAEPELEEELEADHTPHYPEQETEPPLGSCSDVMLS
VEEEGKEDPLPTAASGKRAKRSGSGATNFSLLKQAGDVEENPGPM
ETDTLLLWVLLLWVPGSTGQVQLVQSGGGLVQPGGSLRLSCAASY
FDFDSYEMSWVRQAPGKGLEWIGSIYHSGSTYYNPSLKSRVTISR
DNSKNTLYLQMNTLRAEDTATYYCARVNMDRFDYWGQGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCEVKTDSTDHVKPKETENTKQPSKSCHKPKAIV
HTEKVNMMSLTVLGLRMLFAKTVAVNFLLTAKLFFLRAKRSGSGE
GRGSLLTCGDVEENPGPMETDTLLLWVLLLWVPGSTGQVQLVQSG
GGLVQPGGSLRLSCAASYFDFDSYEMSWVRQAPGKGLEWIGSIYH
SGSTYYNPSLKSRVTISRDNSKNTLYLQMNTLRAEDTATYYCARV
NMDRFDYWGQGTLVTVSSRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGECPIKTDVITMD
PKDNCSKDANDTLLLQLTNTSAYYMYLLLLLKSVVYFAIITCCLL
RRTAFCCNGEKS

| Residues | Feature |
|---|---|
| 1-20 | Mouse IgG kappa signal peptide |
| 21-133 | hYP7 VL domain |
| 156-272 | hYP7 VH domain |
| 21-272 | hYP7 (VL-VH) scFv |
| 273-282 | c-myc tag |
| 283-557 | CD30 (TNFRSF8) fragment |
| 563-584 | P2A peptide |
| 585-604 | Mouse IgG kappa signal peptide |
| 605-720 | HN3 (VHH) |
| 721-827 | Human IgG1 CH1 domain |
| 828-891 | TCR delta chain fragment |
| 897-917 | T2A peptide |
| 918-937 | Mouse IgG kappa signal peptide |
| 938-1053 | HN3 (VHH) |
| 1054-1160 | IgG kappa constant region |
| 1161-1227 | TCR gamma chain fragment |

ART-HN3-HN3 AbTCR + CSR
(SEQ ID NO: 6)
METDTLLLWVLLLWVPGSTGQVQLVQSGGGLVQPGGSLRLSCAAS
YFDFDSYEMSWVRQAPGKGLEWIGSIYHSGSTYYNPSLKSRVTIS
RDNSKNTLYLQMNTLRAEDTATYYCARVNMDRFDYWGQGTLVTVS
SEQKLISEEDLAAATGAPPLGTQPDCNPTPENGEAPASTSPTQSL
LVDSQASKTLPIPTSAPVALSSTGKPVLDAGPVLFWVILVLVVVV
GSSAFLLCHRRACRKRIRQKLHLCYPVQTSQPKLELVDSRPRRSS
TQLRSGASVTEPVAEERGLMSQPLMETCHSVGAAYLESLPLQDAS
PAGGPSSPRDLPEPRVSTEHTNNKIEKIYIMKADTVIVGTVKAEL
PEGRGLAGPAEPELEEELEADHTPHYPEQETEPPLGSCSDVMLSV
EEEGKEDPLPTAASGKRAKRSGSGATNFSLLKQAGDVEENPGPME
TDTLLLWVLLLWVPGSTGQVQLVQSGGGLVQPGGSLRLSCAASYF
DFDSYEMSWVRQAPGKGLEWIGSIYHSGSTYYNPSLKSRVTISRD
NSKNTLYLQMNTLRAEDTATYYCARVNMDRFDYWGQGTLVTVSSA
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKRVEPKSCEVKTDSTDHVKPKETENTKQPSKSCHKPKAIVH
TEKVNMMSLTVLGLRMLFAKTVAVNFLLTAKLFFLRAKRSGSGEG
RGSLLTCGDVEENPGPMETDTLLLWVLLLWVPGSTGQVQLVQSGG
GLVQPGGSLRLSCAASYFDFDSYEMSWVRQAPGKGLEWIGSIYHS
GSTYYNPSLKSRVTISRDNSKNTLYLQMNTLRAEDTATYYCARVN
MDRFDYWGQGTLVTVSSRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECPIKTDVITMDP
KDNCSKDANDTLLLQLTNTSAYYMYLLLLLKSVVYFAIITCCLLR
RTAFCCNGEKS

| Residues | Feature |
|---|---|
| 1-20 | Mouse IgG kappa signal peptide |
| 21-136 | HN3 (VHH) |
| 137-146 | c-myc tag |
| 147-421 | CD30 (TNFRSF8) fragment |
| 427-448 | P2A peptide |
| 449-468 | Mouse IgG kappa signal peptide |
| 469-584 | HN3 (VHH) |
| 585-691 | Human IgG1 CH1 domain |
| 692-755 | TCR delta chain fragment |
| 761-781 | T2A peptide |
| 782-801 | Mouse IgG kappa signal peptide |
| 802-917 | HN3 (VHH) |
| 918-1024 | IgG kappa constant region |
| 1025-1091 | TCR gamma chain fragment |

Human IgG1 CH1 domain
(SEQ ID NO: 17)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCEVKT
Human IgG kappa constant domain
(SEQ ID NO: 18)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
TCRδ chain fragment
(SEQ ID NO: 19)
DSTDHVKPKETENTKQPSKSCHKPKAIVHTEKVNMMSLTVLGLRM
LFAKTVAVNFLLTAKLFFL
TCRγ chain fragment
(SEQ ID NO: 20)
PIKTDVITMDPKDNCSKDANDTLLLQLTNTSAYYMYLLLLLKSVV
YFAIITCCLLRRTAFCCNGEKS
TCRα partial constant region, transmembrane
and intracellular domains
(SEQ ID NO: 47)
IPEDTFFPSPESSCDVKLVEKSFETDTNLNFQNLSVIGFRILLLK
VAGFNLLMTLRLWSS
TCRβ partial constant region, transmembrane
and intracellular domains
(SEQ ID NO: 48)
GRADCGFTSVSYQQGVLSATILYEILLGKATLYAVLVSALVLMAM
VKRKDF
Human CD30 fragment
(SEQ ID NO: 21)
AAATGAPPLGTQPDCNPTPENGEAPASTSPTQSLLVDSQASKTLP
IPTSAPVALSSTGKPVLDAGPVLFWVILVLVVVVGSSAFLLCHRR
ACRKRIRQKLHLCYPVQTSQPKLELVDSRPRRSSTQLRSGASVTE
PVAEERGLMSQPLMETCHSVGAAYLESLPLQDASPAGGPSSPRDL
PEPRVSTEHTNNKIEKIYIMKADTVIVGTVKAELPEGRGLAGPAE
PELEEELEADHTPHYPEQETEPPLGSCSDVMLSVEEEGKEDPLPT
AASGK
CD30 intracellular signaling domain
(SEQ ID NO: 49)
CHRRACRKRIRQKLHLCYPVQTSQPKLELVDSRPRRSSTQLRSGA
SVTEPVAEERGLMSQPLMETCHSVGAAYLESLPLQDASPAGGPSS
PRDLPEPRVSTEHTNNKIEKIYIMKADTVIVGTVKAELPEGRGLA
GPAEPELEEELEADHTPHYPEQETEPPLGSCSDVMLSVEEEGKED
PLPTAASGK
CD28 transmembrane and intracellular signaling
domains
(SEQ ID NO: 50)
IEVMYPPPYLDNEKSNGTIIHVKGKHLCPSPLFPGPSKPFWVLVV
VGGVLACYSLLVTVAFIIFWVRSKRSRLLHSDYMNMTPRRPGPTR
KHYQPYAPPRDFAAYRS
CD28 intracellular signaling domain
(SEQ ID NO: 51)
RSKRSRLLHSDYMNMTPRRPGPTRKHYQPYAPPRDFAAYRS
4-1BB transmembrane and intracellular signaling
domains
(SEQ ID NO: 52)
PADLSPGASSVTPPAPAREPGHSPQIISFFLALTSTALLFLLFFL
TLRFSVVKRGRKKLLYIFKQPFMRPVQTTQEEDGCSCRFPEEEEG
GCEL
4-1BB intracellular signaling domain
(SEQ ID NO: 53)
KRGRKKLLYIFKQPFMRPVQTTQEEDGCSCRFPEEEEGGCEL
Mouse IgG kappa signal peptide
(SEQ ID NO: 22)
METDTLLLWVLLLWVPGSTG
c-Myc tag
(SEQ ID NO: 23)
EQKLISEEDL
P2A peptide
(SEQ ID NO: 24)
GSGATNFSLLKQAGDVEENPGP
T2A peptide
(SEQ ID NO: 25)
GSGEGRGSLLTCGDVEENPGP

### B. Nucleic Acid Sequences

The following sequences are exemplary nucleic acid sequences that can be used as coding sequences for one or more components of an AbTCR or CSR.

CD28 transmembrane and intracellular signaling
domains
(SEQ ID NO: 33)
ATTGAAGTTATGTATCCTCCTCCTTACCTAGACAATGAGAAGAGC
AATGGAACCATTATCCATGTGAAAGGGAAACACCTTTGTCCAAGT
CCCCTATTTCCCGGACCTTCTAAGCCCTTTTGGGTGCTGGTGGTG
GTTGGTGGAGTCCTGGCTTGCTATAGCTTGCTAGTAACAGTGGCC
TTTATTATTTTCTGGGTGAGGAGTAAGAGGAGCAGGCTCCTGCAC
AGTGACTACATGAACATGACTCCCCGCCGCCCCGGGCCCACCCGC
AAGCATTACCAGCCCTATGCCCCACCACGCGACTTCGCAGCCTAT
CGCTCC
CD28 intracellular signaling domain
(SEQ ID NO: 34)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC
4-1BB transmembrane and intracellular signaling
domains
(SEQ ID NO: 35)
CCAGCCGACCTCTCTCCGGGAGCATCCTCTGTGACCCCGCCTGCC
CCTGCGAGAGAGCCAGGACACTCTCCGCAGATCATCTCCTTCTTT
CTTGCGCTGACGTCGACTGCGTTGCTCTTCCTGCTGTTCTTCCTC
ACGCTCCGTTTCTCTGTTGTTAAACGGGGCAGAAAGAAACTCCTG
TATATATTCAAACAACCATTTATGAGACCAGTACAAACTACTCAA
GAGGAAGATGGCTGTAGCTGCCGATTTCCAGAAGAAGAAGAAGGA
GGATGTGAACTG
4-1BB intracellular signaling domain
(SEQ ID NO: 36)
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG
CD30 transmembrane and intracellular signaling
domains
(SEQ ID NO: 37)
GCGCCACCCCTGGGGACCCAGCCGGACTGCAACCCCACCCCAGAG
AATGGCGAGGCGCCTGCCAGCACCAGCCCCACTCAGAGCTTGCTG
GTGGACTCCCAGGCCAGTAAGACGCTGCCCATCCCAACCAGCGCT
CCCGTCGCTCTCTCCTCCACGGGGAAGCCCGTTCTGGATGCAGGG
CCAGTGCTCTTCTGGGTGATCCTGGTGTTGGTTGTGGTGGTCGGC
TCCAGCGCCTTCCTCCTGTGCCACCGGAGGGCCTGCAGGAAGCGA
ATTCGGCAGAAGCTCCACCTGTGCTACCCGGTCCAGACCTCCCAG
CCCAAGCTAGAGCTTGTGGATTCCAGACCCAGGAGGAGCTCAACG
CAGCTGAGGAGTGGTGCGTCGGTGACAGAACCCGTCGCGGAAGAG
CGAGGGTTAATGAGCCAGCCACTGATGGAGACCTGCCACAGCGTG
GGGGCAGCCTACCTGGAGAGCCTGCCGCTGCAGGATGCCAGCCCG
GCCGGGGGCCCCTCGTCCCCCAGGGACCTTCCTGAGCCCCGGGTG
TCCACGGAGCACACCAATAACAAGATTGAGAAAATCTACATCATG
AAGGCTGACACCGTGATCGTGGGGACCGTGAAGGCTGAGCTGCCG
GAGGGCCGGGGCCTGGCGGGGCCAGCAGAGCCCGAGTTGGAGGAG
GAGCTGGAGGCGGACCATACCCCCCACTACCCCGAGCAGGAGACA
GAACCGCCTCTGGGCAGCTGCAGCGATGTCATGCTCTCAGTGGAA
GAGGAAGGGAAAGAAGACCCCTTGCCCACAGCTGCCTCTGGAAAG
CD30 intracellular signaling domain
(SEQ ID NO: 38)
CACCGGAGGGCCTGCAGGAAGCGAATTCGGCAGAAGCTCCACCTG
TGCTACCCGGTCCAGACCTCCCAGCCCAAGCTAGAGCTTGTGGAT
TCCAGACCCAGGAGGAGCTCAACGCAGCTGAGGAGTGGTGCGTCG
GTGACAGAACCCGTCGCGGAAGAGCGAGGGTTAATGAGCCAGCCA
CTGATGGAGACCTGCCACAGCGTGGGGGCAGCCTACCTGGAGAGC
CTGCCGCTGCAGGATGCCAGCCCGGCCGGGGGCCCCTCGTCCCCC
AGGGACCTTCCTGAGCCCCGGGTGTCCACGGAGCACACCAATAAC
AAGATTGAGAAAATCTACATCATGAAGGCTGACACCGTGATCGTG
GGGACCGTGAAGGCTGAGCTGCCGGAGGGCCGGGGCCTGGCGGGG
CCAGCAGAGCCCGAGTTGGAGGAGGAGCTGGAGGCGGACCATACC
CCCCACTACCCCGAGCAGGAGACAGAACCGCCTCTGGGCAGCTGC
AGCGATGTCATGCTCTCAGTGGAAGAGGAAGGGAAAGAAGACCCC
TTGCCCACAGCTGCCTCTGGAAAG
TCRα partial constant region, transmembrane
and intracellular domains
(SEQ ID NO: 39)
ATTCCAGAAGACACCTTCTTCCCCAGCCCAGAAAGTTCCTGTGAT
GTCAAGCTGGTCGAGAAAAGCTTTGAAACAGATACGAACCTAAAC
TTTCAAAACCTGTCAGTGATTGGGTTCCGAATCCTCCTCCTGAAA
GTGGCCGGGTTTAATCTGCTCATGACGCTGCGGCTGTGGTCCAGC
TCRβ B partial constant region, transmembrane
and intracellular domains
(SEQ ID NO: 40)
GGTAGAGCAGACTGTGGCTTTACCTCGGTGTCCTACCAACAAGGG
GTCCTGTCTGCCACCATCCTCTATGAGATCCTACTAGGGAAGGCC
ACCCTGTATGCTGTGCTGGTCAGCGCCCTTGTGTTGATGGCCATG
GTCAAGAGAAAGGATTTC
TCRδ partial constant region, transmembrane
and intracellular domains
(SEQ ID NO: 41)
GATTCTACAGATCACGTAAAACCAAAGGAAACTGAAAACACAAAG
CAACCTTCAAAGAGCTGCCATAAACCCAAAGCCATAGTTCATACC
GAGAAGGTGAACATGATGTCCCTCACAGTGCTTGGGCTACGAATG
CTGTTTGCAAAGACTGTTGCCGTCAATTTTCTCTTGACTGCCAAG
TTATTTTTCTTG
TCRδ transmembrane domain
(SEQ ID NO: 42)
GTGCTTGGGCTACGAATGCTGTTTGCAAAGACTGTTGCCGTCAAT
TTTCTCTTGACTGCCAAGTTATTTTTC
TCRγ partial constant region, transmembrane
and intracellular domains
(SEQ ID NO: 43)
CCCATAAAAACCGATGTCATCACAATGGATCCCAAAGACAATTGG
TCAAAAGATGCAAATGATACACTACTGCTGCAGCTCACAAACACC
TCTGCATATTACATGTACCTCCTCCTGCTCCTCAAGAGTGTGGTC
TATTTTGCCATCATCACCTGCTGTCTGCTTGGAAGAACGGCTTTC
TGCTGCAATGGAGAGAAATCA
TCRγ transmembrane domain
(SEQ ID NO: 44)
TATTACATGTACCTCCTCCTGCTCCTCAAGAGTGTGGTCTATTTT
GCCATCATCACCTGCTGTCTGCTTG
IgG1 CH1 constant region
(SEQ ID NO: 45)
GCCTCCACCAAGGGCCCATCGGTCTTCCCCCTGGCACCCTCCTCC
AAGAGCACCTCTGGGGGCACAGCGGCCCTGGGCTGCCTGGTCAAG
GACTACTTCCCCGAACCGGTGACGGTGTCGTGGAACTCAGGCGCC
CTGACCAGCGGCGTGCACACCTTCCCGGCTGTCCTACAGTCCTCA
GGACTCTACTCCCTCAGCAGCGTGGTGACCGTGCCCTCCAGCAGC
TTGGGCACCCAGACCTACATCTGCAACGTGAATCACAAGCCCAGC
AACACCAAGGTGGACAAAAGAGTTGAACCCAAAAGTTGTGAAGTT
AAAACT
IgGκ light chain constant region
(SEQ ID NO: 46)
AGGACTGTTGCTGCTCCATCTGTTTTTATTTTTCCACCATCTGAT
GAACAACTTAAATCTGGAACTGCTTCTGTTGTTTGTCTTCTTAAT
AATTTTTATCCAAGAGAAGCTAAAGTTCAATGGAAAGTTGATAAT
GCTCTTCAATCTGGAAATTCTCAAGAATCTGTTACTGAACAAGAT
TCTAAAGATTCTACTTATTCTCTTTCTTCTACTCTTACTCTTTCT
AAAGCTGATTATGAAAAACATAAAGTTTATGCTTGTGAAGTTACT
CATCAAGGACTTTCTTCTCCAGTTACTAAATCTTTTAATAGAGGA
GAATGT

### VII. AbTCR and CSR-Expressing Cell Compositions and Administration Thereof

Compositions are provided that include AbTCR and CSR-expressing cells in combination with one or more pharmaceutically or physiologically acceptable carriers, diluents or excipients. The AbTCR+CSR-expressing cells can be iPSCs, T cells (including cytotoxic T cells, T regulatory (Treg) cells, αβT cells, γδT cells, CD3⁺ T cells, CD4⁺ T cells and/or CD8⁺ T cells), NK cells, B cells, mucosal-associated invariant T (MAIT) cells, dendritic cells, macrophages or any other suitable immune cell. Such compositions may include buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. In some examples, the cell-containing composition includes a cryopreservative, such as DMSO or glycerol. In some examples the cell-containing composition includes a culture media, such as DMEM or RPMI, and may further include serum, such as FBS. In some examples, the cell-containing composition is frozen or in a liquid form. The cells can be autologous to the recipient. However, the cells can also be heterologous (allogeneic).

With regard to the cells, a variety of aqueous carriers can be used, for example, buffered saline and the like, for introducing the cells. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject's needs.

The precise amount of the composition to be administered can be determined by a physician with consideration of individual differences in age, weight, tumor size/burden, extent of metastasis, and condition of the patient (subject). It can generally be stated that a pharmaceutical composition that includes the AbTCR+CSR-expressing immune cells or iPSCs described herein may be administered at a dosage of 10⁴ to 10⁹ cells/kg body weight, such as 10⁵ to 10⁶ cells/kg body weight, including all integer values within those ranges. Exemplary doses are 10⁶ cells/kg to about 10⁸ cells/kg, such as from about 5×10⁶ cells/kg to about 7.5×10⁷ cells/kg, such as at about 2.5×10⁷ cells/kg, or at about 5.0×10⁷ cells/kg.

A composition can be administered once or multiple times, such as 2, 3, 4, 5, 6, 7, 8, 9 or 10 times at these dosages. The composition can be administered using known immunotherapy infusion techniques (see, e.g., Rosenberg et al., New Eng. J. of Med. 319:1676, 1988). The compositions can be administered daily, weekly, bimonthly or monthly. In some non-limiting examples, the composition is formulated for intravenous administration and is administered multiple times. The quantity and frequency of administration can be determined by such factors as the condition of the subject, and the type and severity of the subject's disease, although appropriate dosages may be determined by clinical trials.

In some aspects, the AbTCR+CSR-encoding nucleic acid molecule is introduced into cells, such as immune cells or iPSCs, and the subject receives an initial administration of cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In one aspect, more than one administration of the AbTCR+CSR-expressing cells are administered to the subject per week, e.g., 2, 3, or 4 administrations of the AbTCR+CSR-expressing cells of the disclosure are administered per week. In one aspect, the subject receives more than one administration of the AbTCR+CSR-expressing cells per week (e.g., 2, 3 or 4 administrations per week) (also referred to as a cycle), followed by a week of no AbTCR+CSR-expressing cell administrations, and then one or more additional administration of the AbTCR+CSR-expressing cells (e.g., more than one administration of the AbTCR+CSR-expressing cells per week) is administered to the subject. In another aspect, the subject (e.g., a human subject) receives more than one cycle of AbTCR+CSR-expressing cells, and the time between each cycle is less than 10, 9, 8, 7, 6, 5, 4, or 3 days. In one aspect, the AbTCR+CSR-expressing cells are administered every other day for 3 administrations per week. In another aspect, the AbTCR+CSR-expressing cells are administered for at least two, three, four, five, six, seven, eight or more weeks. The dosage of the above treatments to be administered to a patient will vary with the precise nature of the condition being treated and the recipient of the treatment. The scaling of dosages for human administration can be performed according to accepted practices.

In some aspects, AbTCR+CSR-expressing cells are able to replicate in vivo resulting in long-term persistence that can lead to sustained tumor control. In various aspects, the immune cells or iPSCs administered to the subject, or the progeny of these cells, persist in the subject for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, or for years after administration of the cells to the subject. In other aspects, the cells and their progeny are present for less than six months, five months, four months, three months, two months, or one month, e.g., three weeks, two weeks, one week, after administration of the AbTCR+CSR-expressing cells to the subject.

The administration of the disclosed compositions may be carried out in any convenient manner, including by injection, ingestion, transfusion, implantation or transplantation. The disclosed compositions can be administered to a patient trans-arterially, subcutaneously, intradermally, intratumorally, intranodally, intramedullary, intracerebrally, intraventricularly, intracranially, intramuscularly, intra-arterially (including into the hepatic artery (such as HAI) or the femoral artery), by intravenous (i.v.) injection, intraprostatically (e.g., for a prostate cancer), intraosseously, intravitreally, or intraperitoneally. In some aspects, the compositions are administered to a patient by intradermal or subcutaneous injection. In other aspects, the compositions of the disclosure are administered by i.v. injection. In other aspects, the compositions of the disclosure are administered by intra-arterial injection. The compositions can also be injected directly into a tumor or lymph node. In one example, administration is intraosseous and the cancer treated is a cancer of the bone (e.g., osteosarcoma). In one example, administration is intracerebral, intraventricular, or intracranial and the cancer treated is a cancer of the brain (e.g., neuroblastoma or medulloblastoma). In one example, administration is intravitreal and the cancer treated is a cancer of the eye (e.g., retinoblastoma).

In some aspects, subjects can undergo leukapheresis, wherein leukocytes are collected, enriched, or depleted ex vivo to select and/or isolate the cells of interest, e.g., T cells, macrophages, NK cells and/or immune cells. These cell isolates may be expanded by known methods and treated such that one or more AbTCR+CSR constructs can be introduced, thereby creating an autologous cell that expresses the AbTCR+CSR.

In some aspects, immune cells (such as T cells, NK cells and/or macrophages) are isolated from peripheral blood by lysing the red blood cells and in some instances depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells, see U.S. Published Application No. US20140271635. In a non-limiting example, the time period is about 30 minutes. In other non-limiting examples, the time period ranges from 30 minutes to 36 hours or longer and all integer values there between. In further non-limiting examples, the time period is at least 1, 2, 3, 4, 5, or 6 hours, 10 to 24 hours, 24 hours or longer. Longer incubation times can be used to isolate T cells in any situation where there are few T cells as compared to other cell types, such as in isolation from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. Multiple rounds of selection can also be used.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ T cells by negative selection, a monoclonal antibody cocktail typically includes antibodies to CD14, CD20, CD11b, CD16, HLA-DR, and CD8. A T cell population can be selected that expresses one or more cytokines. Methods for screening for cell expression are disclosed in PCT Publication No. WO 2013/126712.

For isolation of a desired population of cells by positive or negative selection, the concentration of cells and surface (e.g., particles such as beads) can be varied to ensure maximum contact of cells and beads. In some aspects, a concentration of 1 billion cells/ml is used. In further aspects, greater than 100 million cells/ml is used. In other aspects, a concentration of cells of 10, 15, 20, 25, 30, 35, 40, 45, 50, 55, 60, 65, 70, 75, 80, 85, 90, 95, or 100 million cells/ml is used. Without being bound by theory, using high concentrations can result in increased cell yield, cell activation, and cell expansion. Lower concentrations of cells can also be used. Without being bound by theory, by significantly diluting the mixture of T cells and surface (e.g., particles such as beads), interactions between the particles and cells can be minimized. This selects for cells that express high amounts of desired antigens to be bound to the particles. For example, CD4+ T cells express higher levels of CD28 and are more efficiently captured than CD8⁺ T cells in dilute concentrations. In some aspects, the concentration of cells used is 5×10⁶/ml. In other aspects, the concentration used can be from about 1×10⁵/ml to 1×10⁶/ml, and any integer value in between.

### VIII. Methods of Treatment

Provided herein are methods of treating GPC2-positive or GPC3-positive cancer in a subject by administering to the subject a therapeutically effective amount of a GPC2-targeted or GPC3-targeted AbTCR+CSR-expressing immune cell (such as, but not limited to, T cell, NK cell or macrophage) or AbTCR+CSR-expressing iPSC as disclosed herein. Also provided herein is a method of inhibiting tumor growth or metastasis in a subject by administering to the subject a therapeutically effective amount of a GPC2-targeted or GPC3-targeted AbTCR+CSR-expressing cell disclosed herein. Thus, in some examples, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment. In some examples, the methods increase the survival time of a subject with a GPC2-positive cancer or a GPC3-positive cancer by at least 3 months, at least 6 months, at least 9 months, at least 12 months, at least 18 months, at least 24 months, at last 36 months, at least 48 months, or at least 60 months, for example relative to the survival time in an absence of the treatment provided herein. In some examples, combinations of these effects are achieved.

Specifically provided is a method of treating a GPC2-positive or GPC3-positive cancer in a subject. In some aspects, the method includes administering to the subject a therapeutically effective amount of an isolated immune cell or iPSC that includes a nucleic acid molecule encoding a GPC2-targeted AbTCR+CSR or a GPC3-targeted AbTCR+CSR, or administering a therapeutically effective amount of an isolated immune cell or iPSC expressing a GPC2-targeted or GPC3-targeted AbTCR+CSR. In some aspects, the GPC2-positive cancer is a solid tumor. In specific examples, the GPC2-positive cancer is neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma, small cell lung cancer, or osteosarcoma. In other specific examples, the GPC3-positive cancer is hepatocellular carcinoma (HCC), melanoma, ovarian clear-cell carcinoma, yolk sac tumor (YST), hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma, salivary gland cancer, glioblastoma, choriocarcinoma, rhabdosarcoma, renal clear-cell carcinoma, or embryonal tumor.

In some aspects of the methods disclosed herein, the isolated immune cells are T lymphocytes. In some examples, the T lymphocytes are autologous T lymphocytes. In other aspects, the isolated host cells are NK cells, NK T cells, MAIT cells, DCs, macrophages or B cells.

A therapeutically effective amount of an AbTCR+CSR-expressing immune cell or iPSC can depend upon the severity of the disease, the type of disease, and the general state of the patient's health. A therapeutically effective amount of AbTCR+CSR-expressing cells and compositions thereof is that which provides either subjective relief of a symptom(s) or an objectively identifiable improvement as noted by the clinician or other qualified observer (such as a decrease in tumor volume or metastasis).

Administration of the AbTCR+CSR-expressing cells and compositions disclosed herein can also be accompanied by administration of other anti-cancer agents or therapeutic treatments (such as surgical resection of a tumor). Any suitable anti-cancer agent can be administered in combination with the compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-CLTA4, anti-EGFR, or anti-VEGF). In one example, a cancer is treated by administering a GPC2-targeted or GPC3-targeted AbTCR+CSR-expressing immune cell or iPSC disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), or CLTA-4 antibody (e.g., ipilimumab or tremelimumab). In one example, a cancer is treated by administering a GPC2-targeted or GPC3-targeted AbTCR+CSR-expressing cell disclosed herein and one or more mAbs, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

In one example, a cancer is treated by administering a GPC2-targeted or GPC3-targeted AbTCR+CSR-expressing cell (such as immune cell or iPSC) disclosed herein and one or more alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). In one example, a cancer is treated by administering a GPC2-targeted or GPC3-targeted AbTCR+CSR-expressing cell (such as immune cell or iPSC) disclosed herein and cyclophosphamide.

In one example, a cancer is treated by administering a GPC2-targeted or GPC3-targeted AbTCR+CSR-expressing cell (such as immune cell or iPSC) disclosed herein and one or more antimetabolites, such as folic acid analogs (such as methotrexate), pyrimidine analogs (such as 5-FU or cytarabine), and purine analogs, such as mercaptopurine or thioguanine.

In one example, a cancer is treated by administering a GPC2-targeted or GPC3-targeted AbTCR+CSR-expressing cell (such as immune cell or iPSC) disclosed herein and one or more natural products, such as include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

In one example, a cancer is treated by administering a GPC2-targeted or GPC3-targeted AbTCR+CSR-expressing cell (such as immune cell or iPSC) disclosed herein and one or more platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

In one example, a cancer is treated by administering a GPC2-targeted or GPC3-targeted AbTCR+CSR-expressing cell (such as immune cell or iPSC) disclosed herein and one or more hormones or antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone).

In one example, a cancer is treated by administering a GPC2-targeted or GPC3-targeted AbTCR+CSR-expressing cell (such as immune cell or iPSC) disclosed herein and one or more chemotherapy drugs, such as Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. In one example, a cancer is treated by administering a GPC2-targeted or GPC3-targeted AbTCR+CSR-expressing cell (such as immune cell or iPSC) disclosed herein, cyclophosphamide and fludarabine. In one example, a cancer is treated by administering a GPC2-targeted or GPC3-targeted AbTCR+CSR-expressing cell (such as immune cell or iPSC) disclosed herein and one or more immunomodulators, such as AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor). Another treatment that can be used in combination with those provided herein is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

### EXAMPLES

The following examples are provided to illustrate particular features of certain aspects of the disclosure, but the scope of the claims should not be limited to those features exemplified.

### Example 1: GPC2-Specific AbTCR+CSR T Cells Kill GPC2-Expressing Neuroblastoma Cells In Vitro

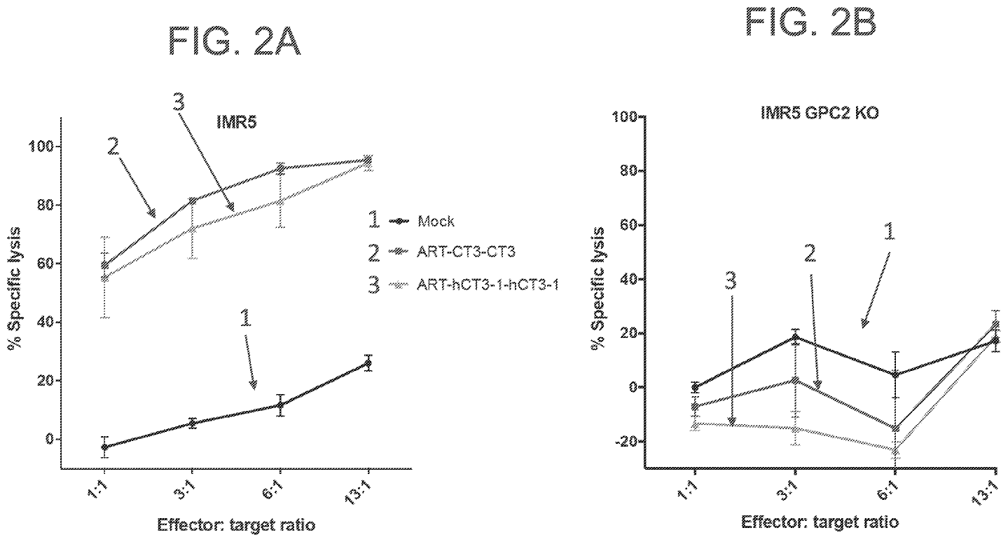

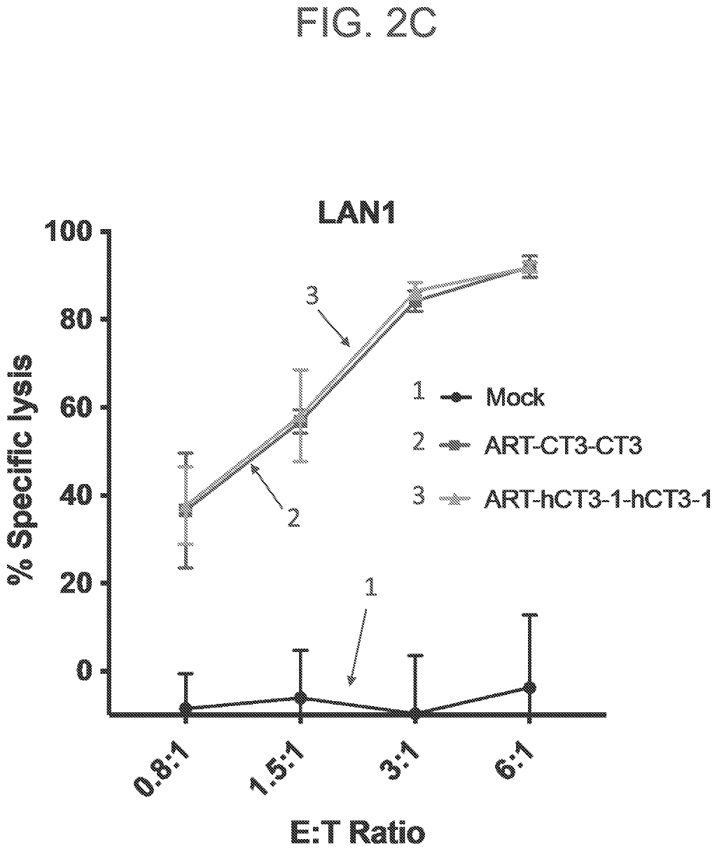

In vitro cell killing of T cells expressing CT3 or hCT3-1 AbTCR+CSRs (FIG. 1A) was evaluated. GPC2-targeted AbTCR+CSR T cells were co-cultured with GPC2-positive neuroblastoma IMR5 cells, GPC2-knockout (KO) IMR5 cells, or GPC2-positive LAN1 cells for 20 hours. Both CT3 and hCT3-1 AbTCR+CSR T cells potently lysed GPC2-positive IMR5 (FIG. 2A) and LAN1 (FIG. 2C) cells at all tested effector to target (E:T) ratios. Neither of the GPC2-targeted AbTCR+CSR T cells killed GPC2 KO IMR5 cells (FIG. 2B), demonstrating the antigen specificity of the AbTCR+CSR T cells.

### Example 2: GPC2-Targeted AbTCR+CSR T Cells Regress Established Tumors in a Metastatic Neuroblastoma Mouse Model

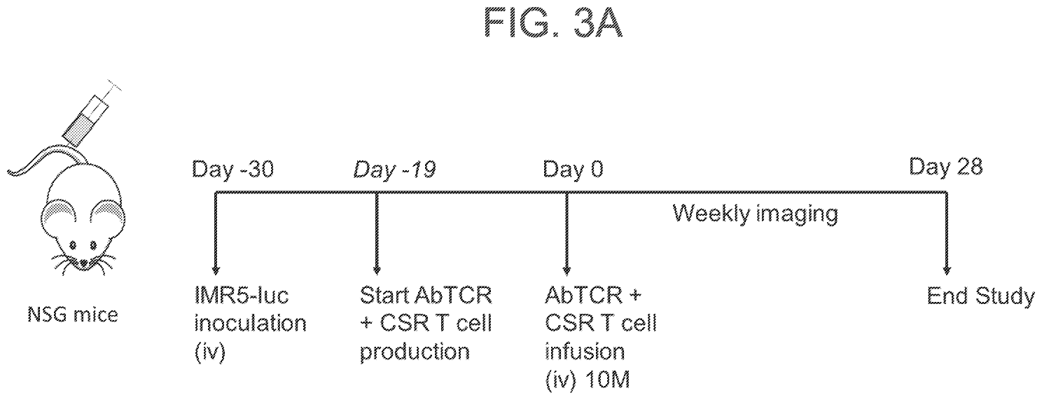

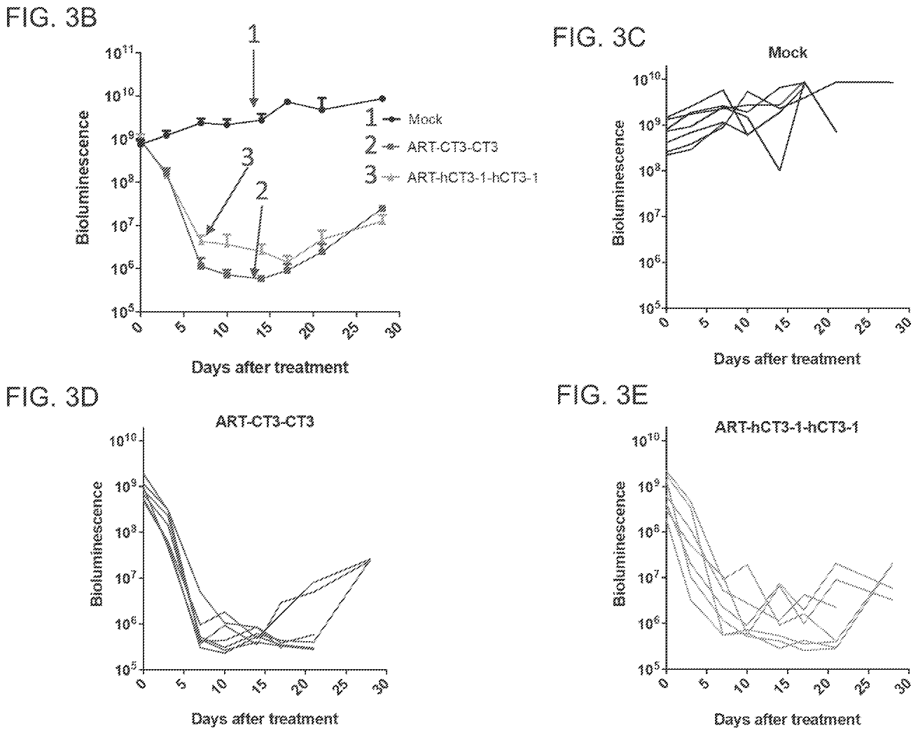

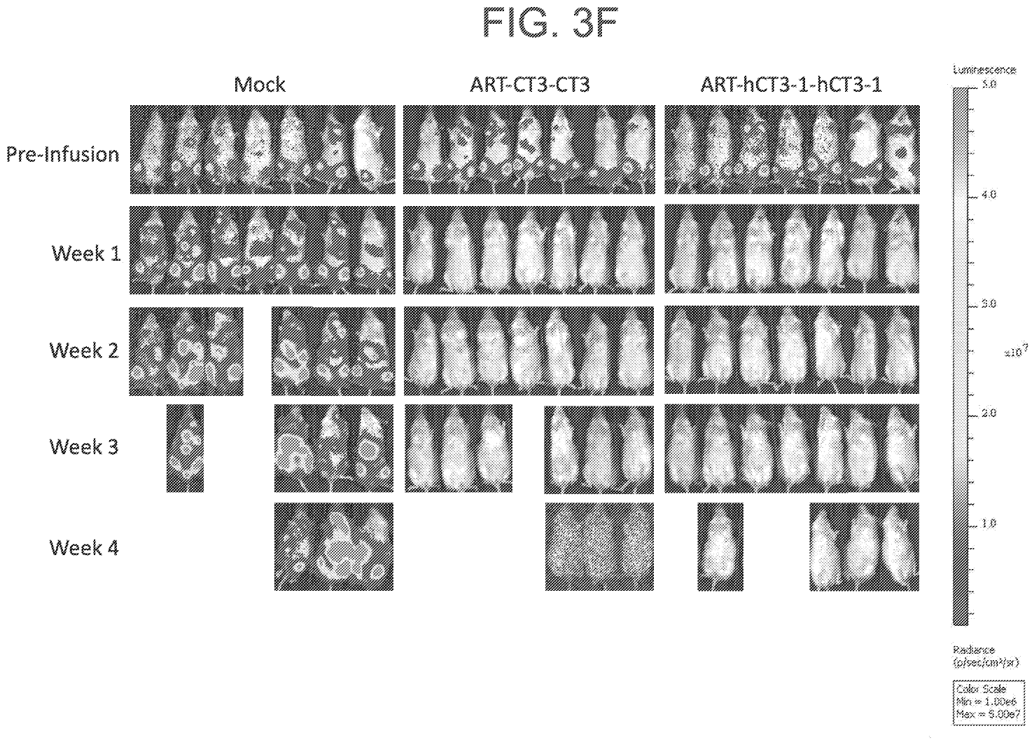

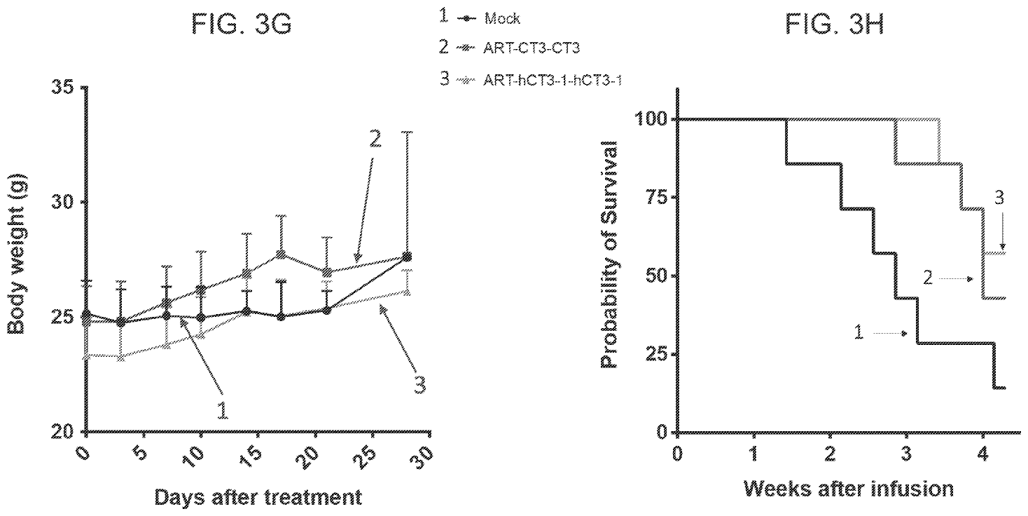

IMR5-Luc cells were injected into the tail vein of study animals thirty days prior to infusion of 10 million AbTCR+CSR T cells (FIG. 3A). Animals were imaged weekly for four weeks. FIG. 3B shows the average bioluminescence of mock-treated mice and mice treated with GPC2-targeted AbTCR+CSR T cells (ART-CT3-CT3 or ART-hCT3-1-hCT3-1). Both CT3 and hCT3-1 AbTCR+CSR T cells rapidly induced regression of established neuroblastoma tumors within the first week post-treatment. Bioluminescence levels of individual mock-treated animals or animals treated with CT3 AbTCR+CSR T cells or hCT3-1 AbTCR+CSR T cells are shown in FIGS. 3C-3E. Treatment with either CT3 or hCT3-1 AbTCR+CSR T cells completely regressed established neuroblastoma tumors in mice, whereas all mock-treated mice exhibited large tumors (FIG. 3F). Body weights of mock-treated mice and mice treated with CT3 or hCT3 AbTCR+CSR T cells were similar among the three groups (FIG. 3G). Probability of survival of mock-treated mice and mice treated with CT3 or hCT3 AbTCR+CSR T cells is shown in FIG. 3H. Treatment with either CT3 or hCT3 AbTCR+CSR T cells improved the probability of survival compared mock-treated animals.

### Example 3: Binding of GPC2-Targeted CARs and AbTCRs to GPC2

Flow cytometry was performed to demonstrate binding of the CT3 and hCT3 based CARs and AbTCRs to GPC2. Shown in FIG. 12 is a flow cytometric analysis of Jurkat T cells expressing CT3 and hCT3 CARs (CT3-CD28HTM and hCT3-1 CD28HTM, respectively) and AbTCRs+CSRs (ART-CT3-CT3 and ART-hCT3-1-hCT3-1, respectively). T cells expressing each of the constructs were capable of binding to GPC2 antigen. Mock T cells not expressing a CAR or AbTCR+CSR were included as a negative control.

### Example 4: Potency of GPC2-Targeted CAR and AbTCR+CSR T Cells in an IMR-5 Mouse Xenograft Model

IMR-5 xenograft mice were i.v. infused with 5 million CAR (CT3-CD28HTM and hCT3-CD28HTM), AbTCR+CSR (ART-CT3-CT3 and ART-hCT3-hCT3) or mock T cells 28 days after tumor inoculation with IMR-5-luc cells (FIG. 13A). Representative bioluminescence images of IMR-5 tumor growth and tumor bioluminescence as photons per second in CAR and AbTCR+CSR T cell treated mice are shown in FIGS. 13B-13C. A Kaplan-Meier survival curve of mice after infusion is shown in FIG. 13D. Treatment with 5 million AbTCR+CSR (ART-CT3-CT3 and ART-hCT3-hCT3) T cells showed better anti-tumor activity, and increased survival, compared to treatment with CAR (CT3-CD28HTM and hCT3-CD28HTM) T cells. ART-hCT3-hCT3 was the most potent in this study.

A tumor re-challenge study was also performed in which luciferase-expressing IMR-5 tumor cells were inoculated i.v. into three mice after 32 days of treatment with ART-hCT3-hCT3. Representative bioluminescence images of the IMR-5 tumor rechallenge are shown in FIG. 13E. These mice remained tumor-free 5 weeks after tumor rechallenge while all of the untreated mice showed growth of IMR-5 tumors (FIG. 13F).

### Example 5: GPC3 Binding of hYP7 and HN3 AbTCR+CSRs

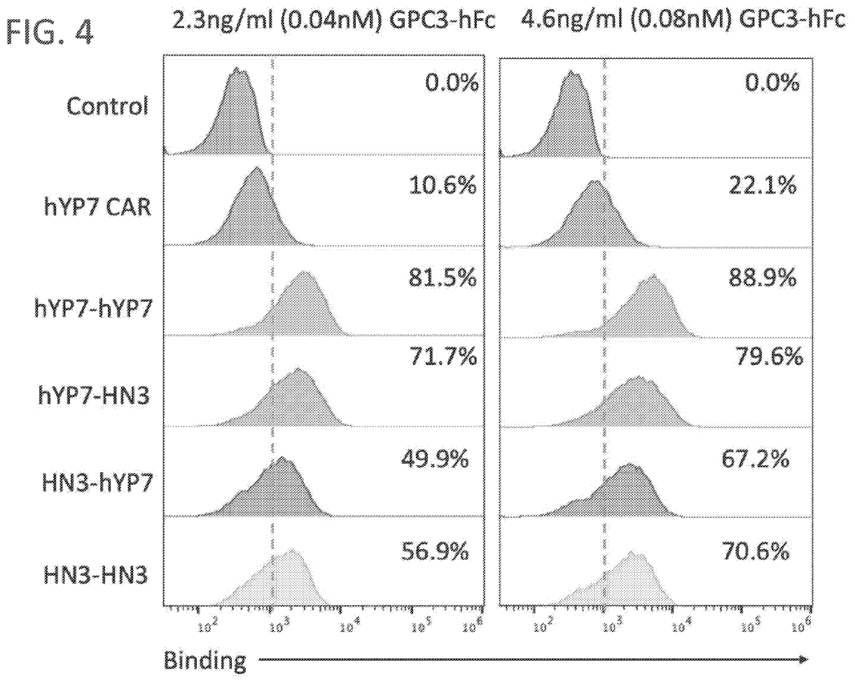

Binding of Jurkat T cells expressing hYP7 CAR or one of four different GPC3-targeted AbTCR+CSRs (hYP7-hYP7, hYP7-HN3, HN3-HN3, and HN3-hYP7; see FIG. 1B) was evaluated by flow cytometric analysis. Binding affinity was tested using GPC3 protein concentrations of 0.04 nM and 0.08 nM. As shown in FIG. 4, T cells expressing any one of the GPC3-targeted AbTCR+CSRs exhibited higher levels of binding than hYP7 CAR T cells.

### Example 6: GPC3-Targeted AbTCR+CSR T Cells Induce Cytotoxicity Against the Hep3B Liver Cancer Cell Line

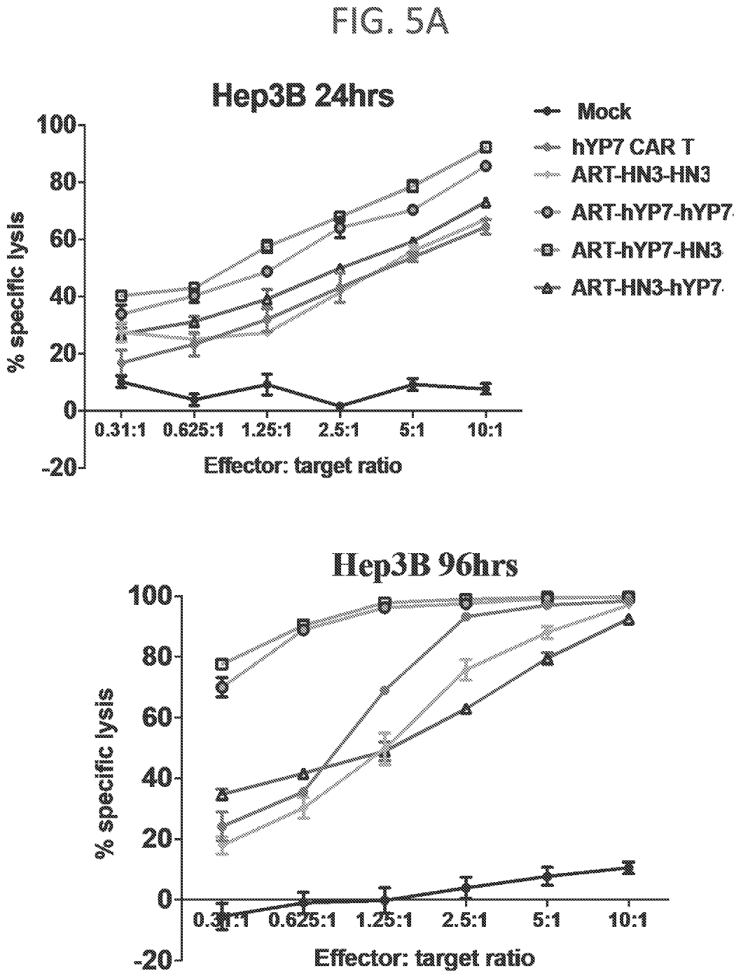

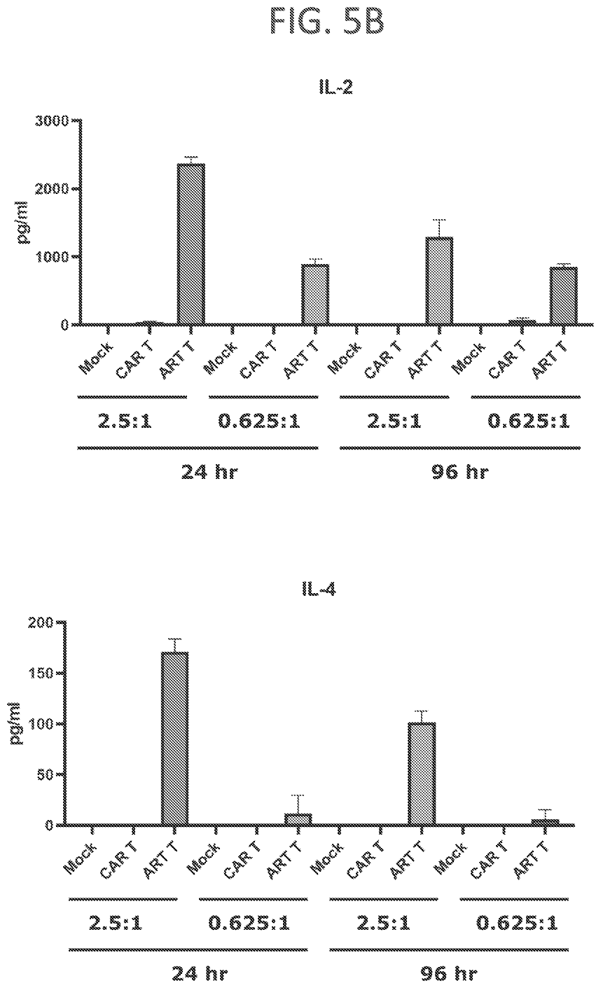

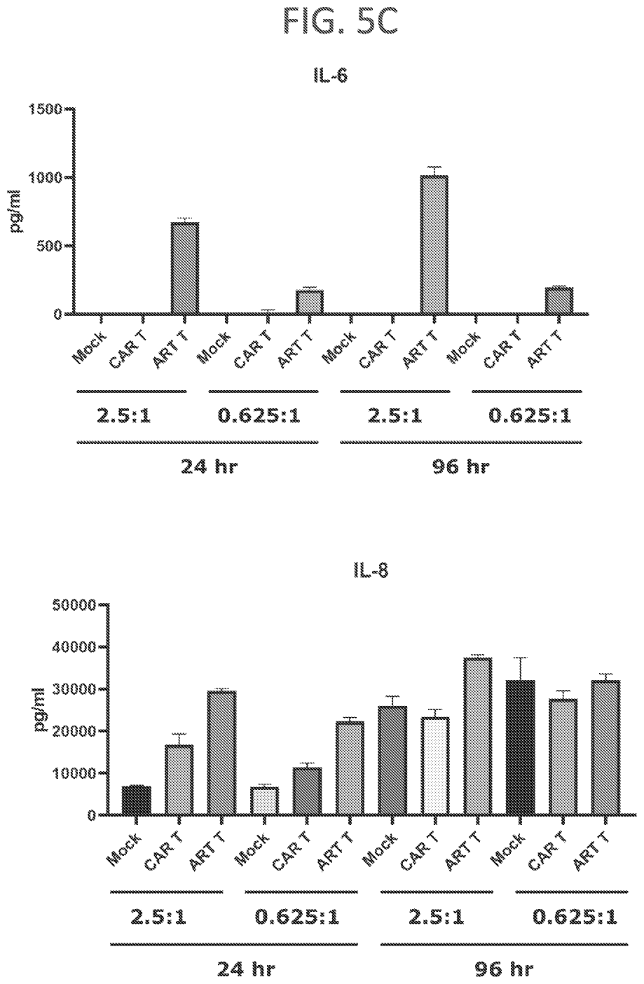

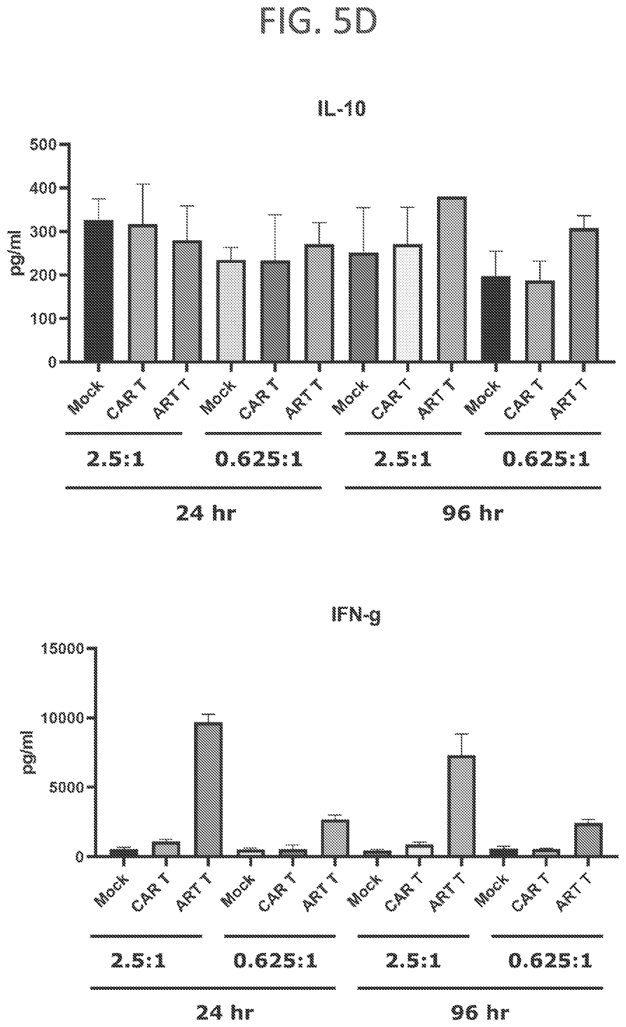

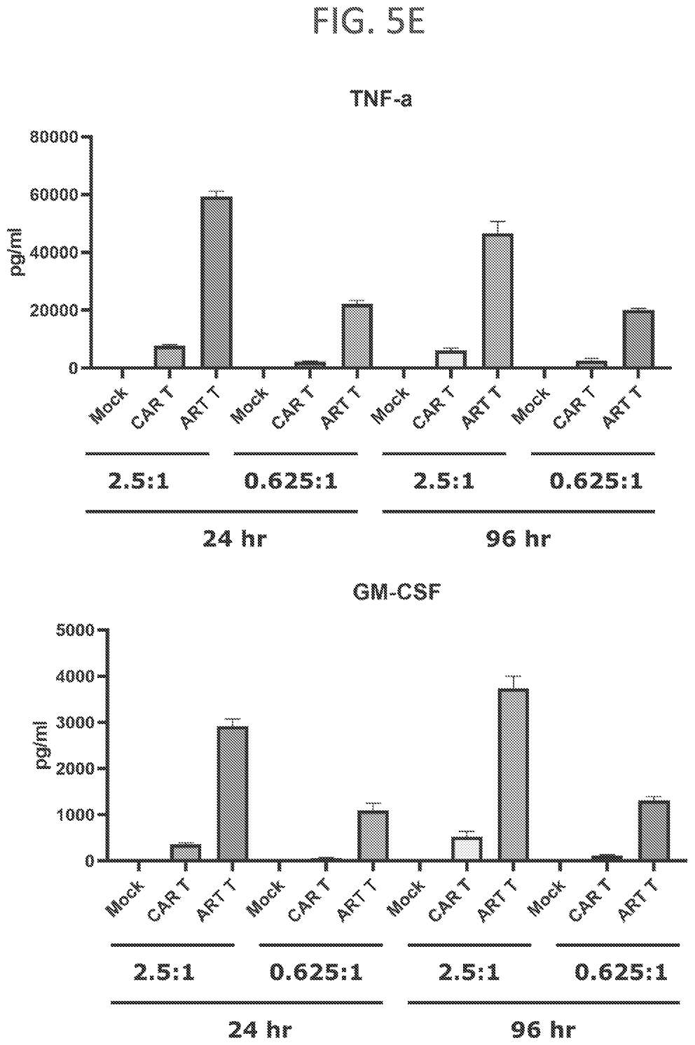

Cytolytic activity of GPC3-targeted CAR (hYP7) T cells and AbTCR+CSR T cells after 24 or 96 hours of incubation with Hep3B cells is shown in FIG. 5A. hYP7-hYP7 and hYP7-HN3 AbTCR+CSR T cells were the most potent. The concentration of cytokines IL-2, IL-4, IL6-, IL-8, IL-10, TNF-α, IFN-γ, and GM-CSF in the supernatants of CAR (hYP7) and AbTCR+CSR (hYP7-hYP7) cytotoxicity assays at an E:T ratio of 5:1 (as in FIG. 5A) was measured by ELISA (FIGS. 5B-5E). For the majority of cytokines measured, hYP7-hYP7 AbTCR+CSR T cells induced higher levels of cytokine production relative to hYP7 CAR T cells.

### Example 7: Inhibition of Wnt/β-Catenin Signaling and Activation of NFAT Signaling by GPC3-Targeted CAR and AbTCR+CSRs T Cells

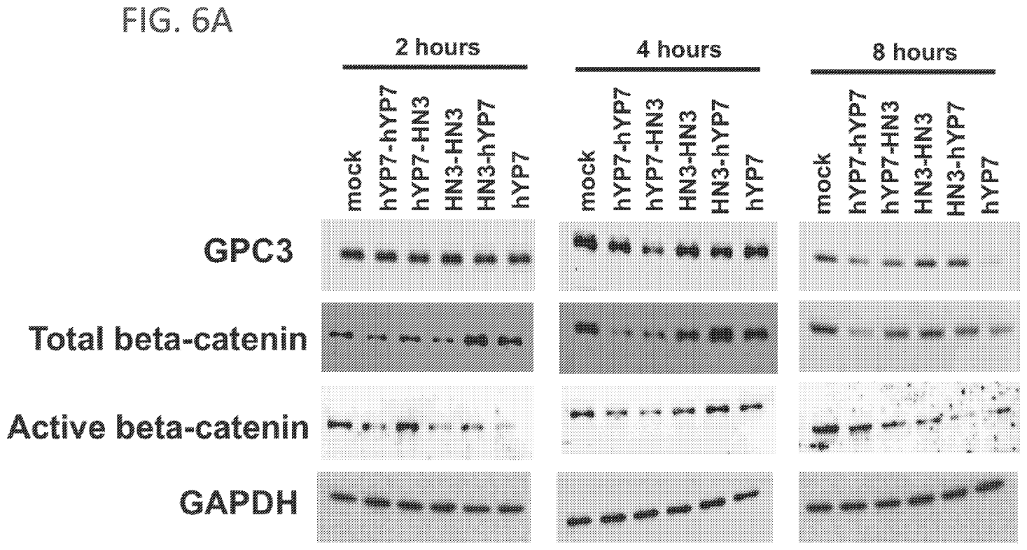

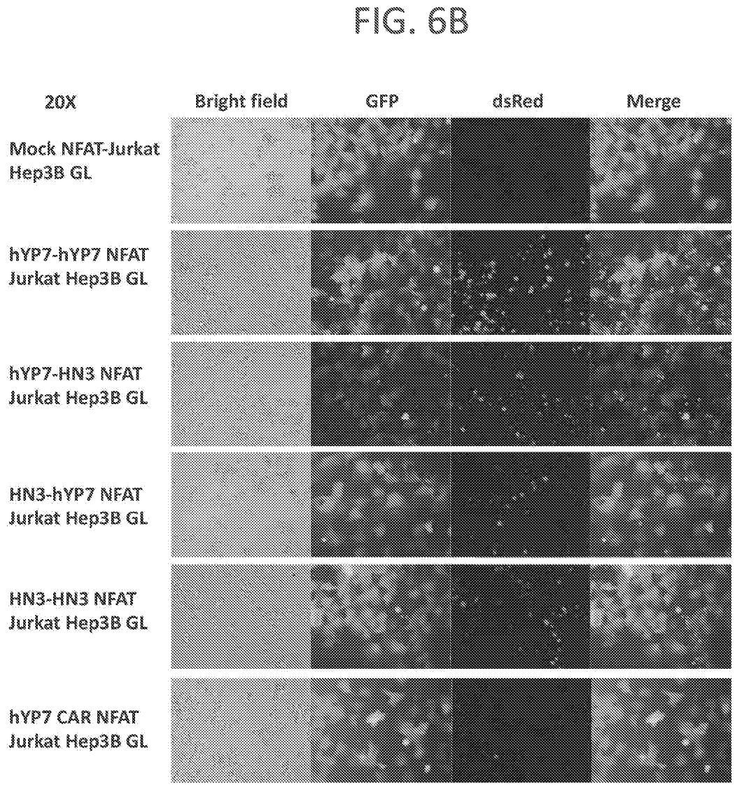

Expression of β-catenin in Hep3B cells cultured with CAR (hYP7) T cells or AbTCR+CSR T cells was measured by Western blot (FIG. 6A). CAR (hYP7) T cells and AbTCR+CSR T cells suppressed the expression of β-catenin in Hep3B cells after 2, 4, and 8 hours. Fluorescence microscopy images of Hep3B co-cultured with NFAT-Jurkat-tdTomato reporter cells transfected with hYP7 CAR and AbTCR+CSR constructs are shown in FIG. 6B. AbTCR+CSR (hYP7-hYP7) NFAT-Jurkat cells showed the greatest NFAT signal, followed by AbTCR+CSR (hYP7-HN3) NFAT-Jurkat, AbTCR+CSR (HN3-hYP7) NFAT-Jurkat, AbTCR+CSR (HN3-HN3) NFAT-Jurkat, and CAR (hYP7) NFAT-Jurkat (FIG. 6B).

### Example 8: Potency of GPC3-Targeted CAR and AbTCR+CSR T Cells in a Hep3B Mouse Xenograft Model

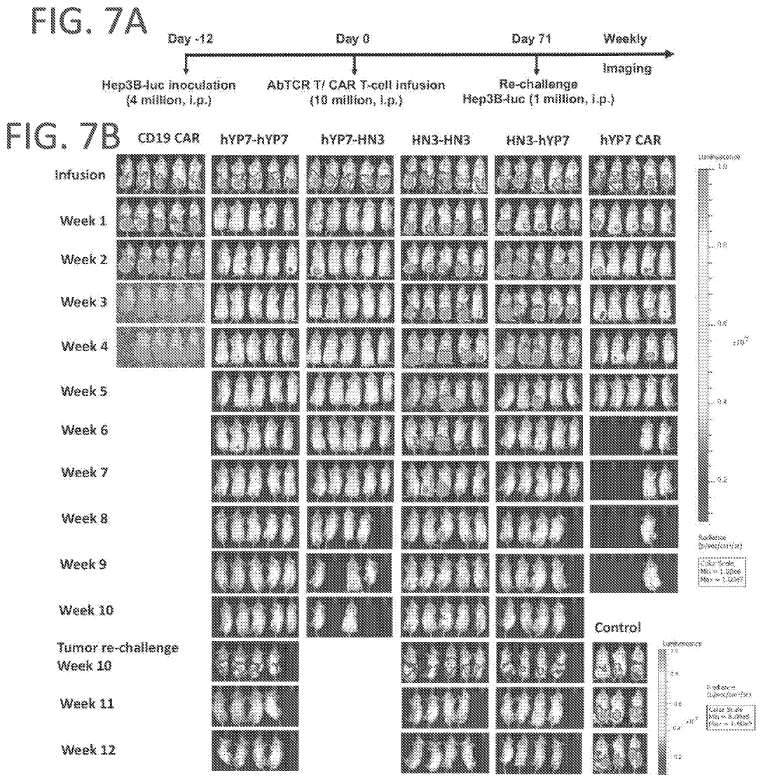

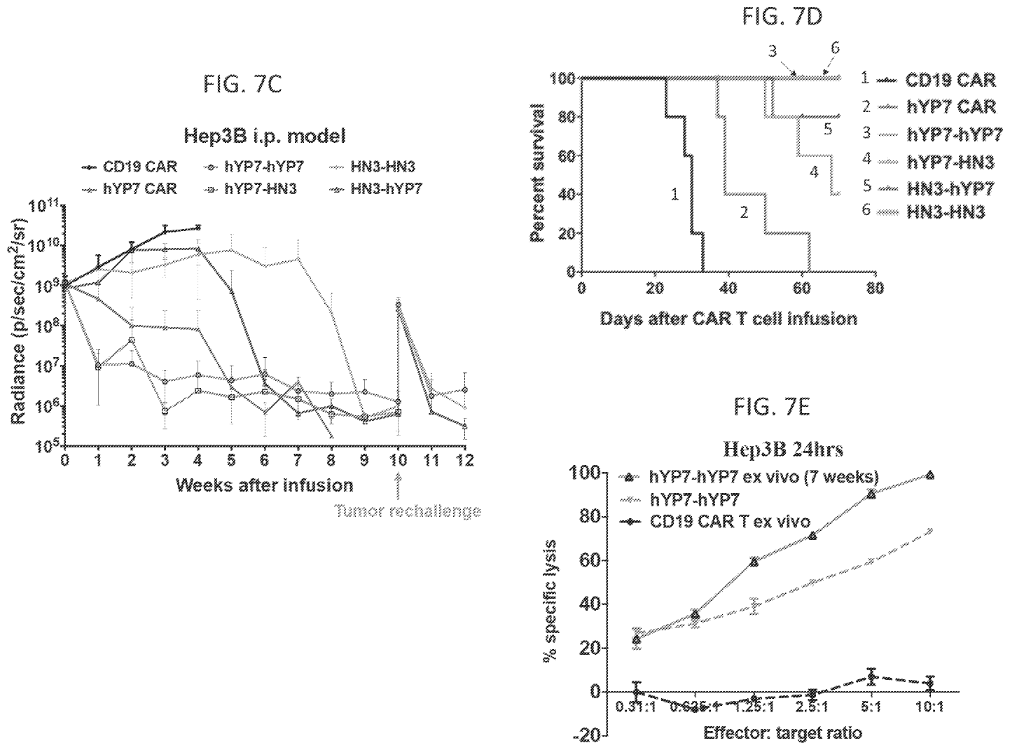

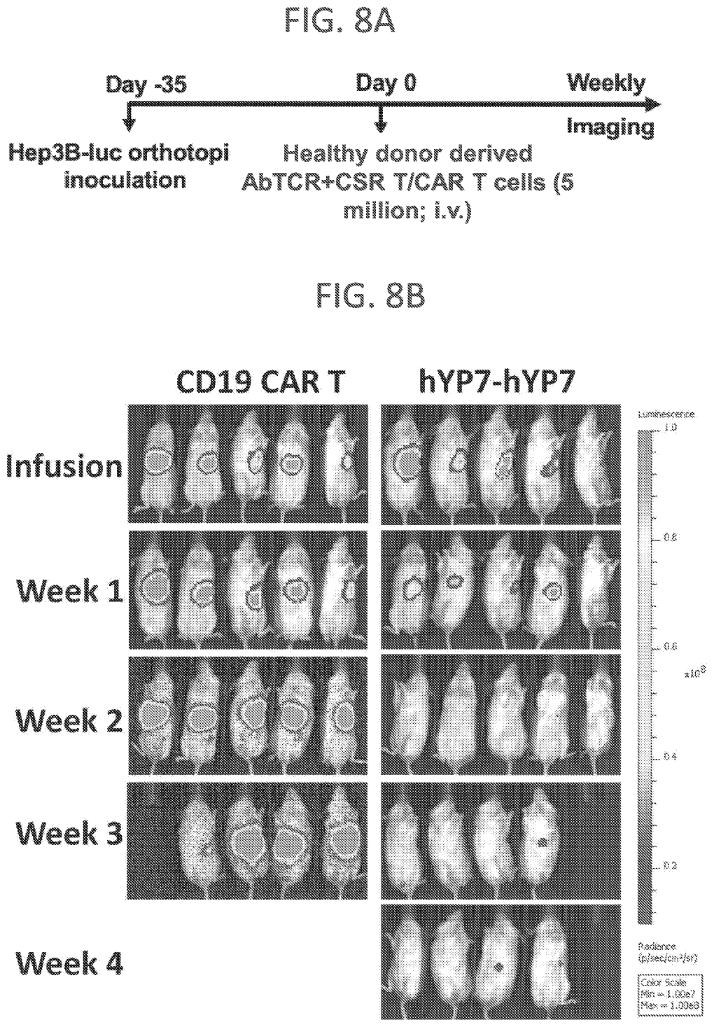

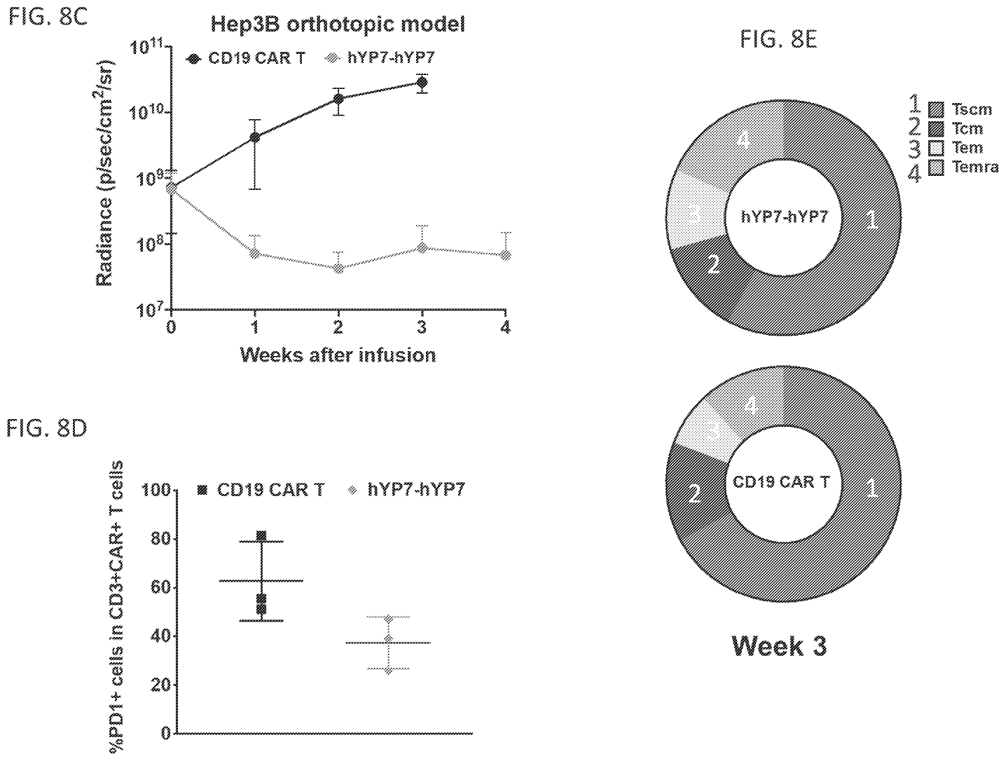

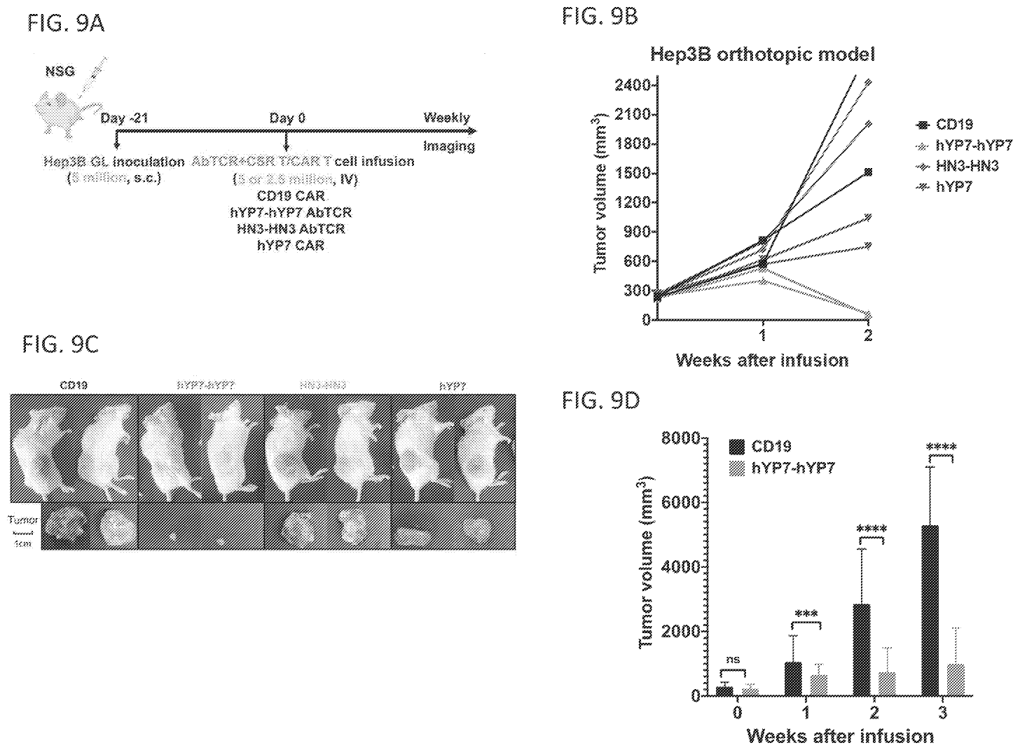

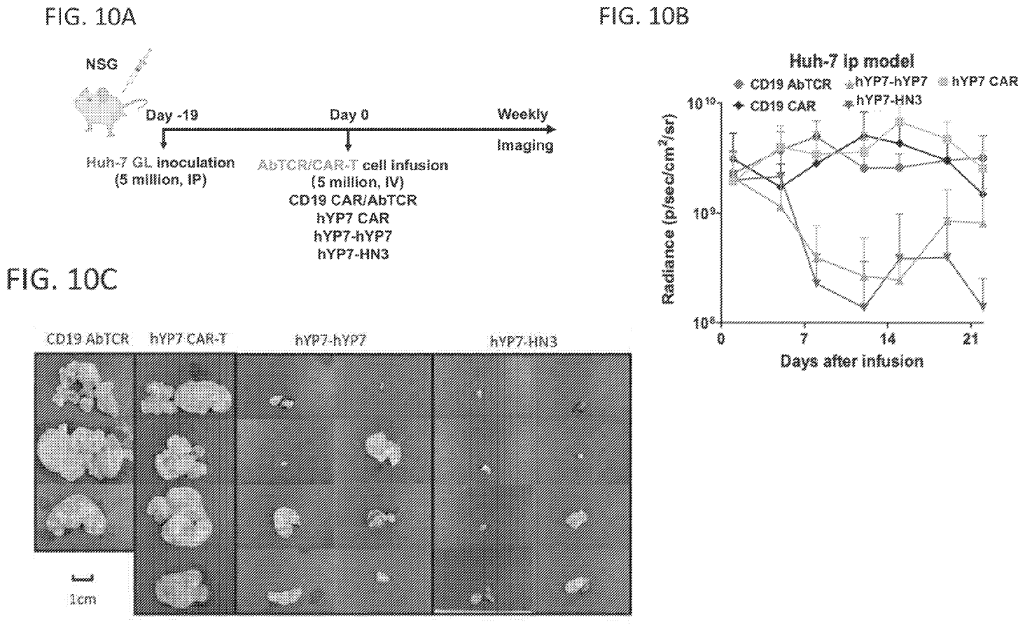

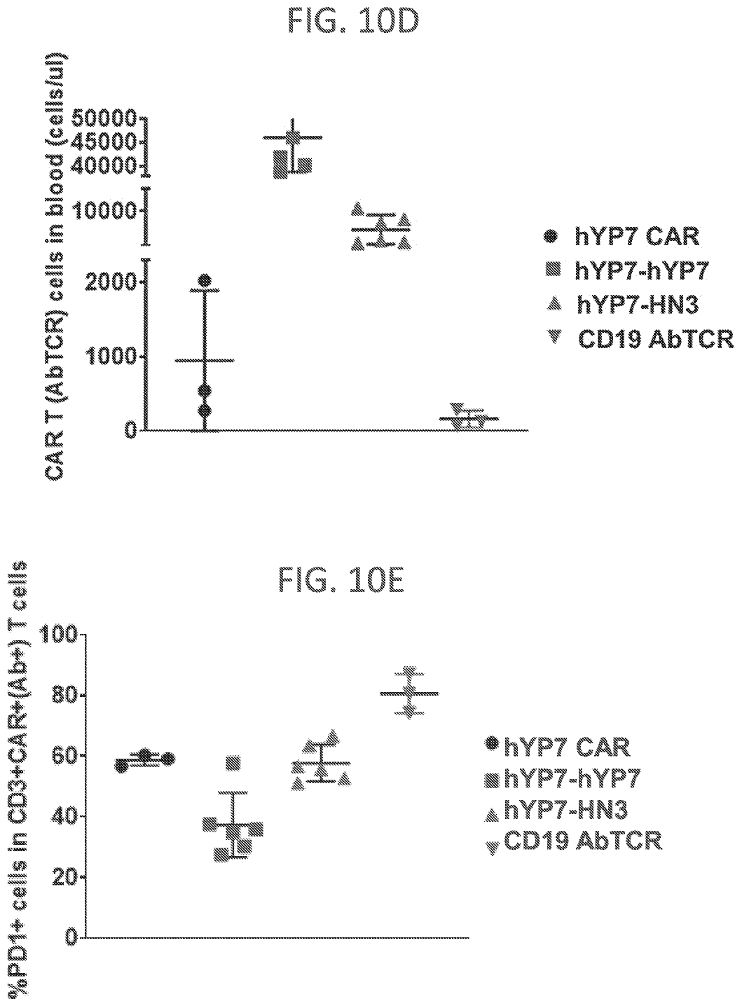

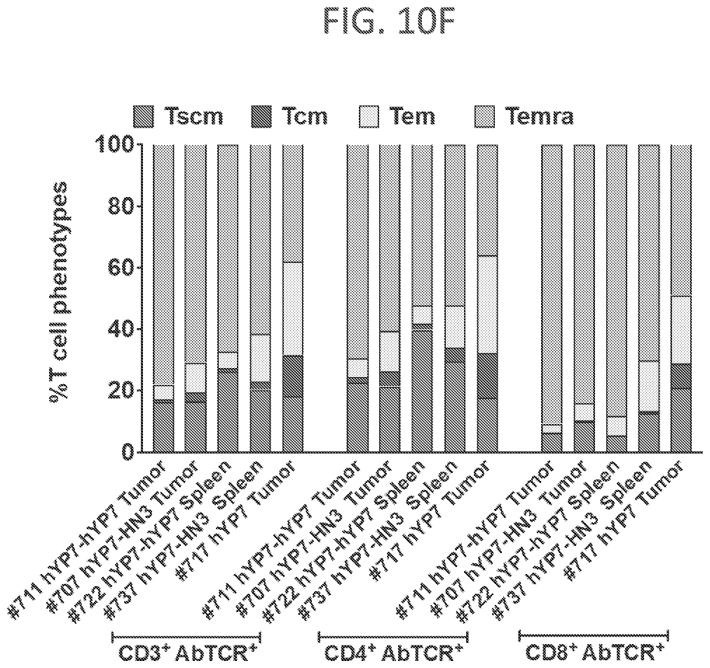

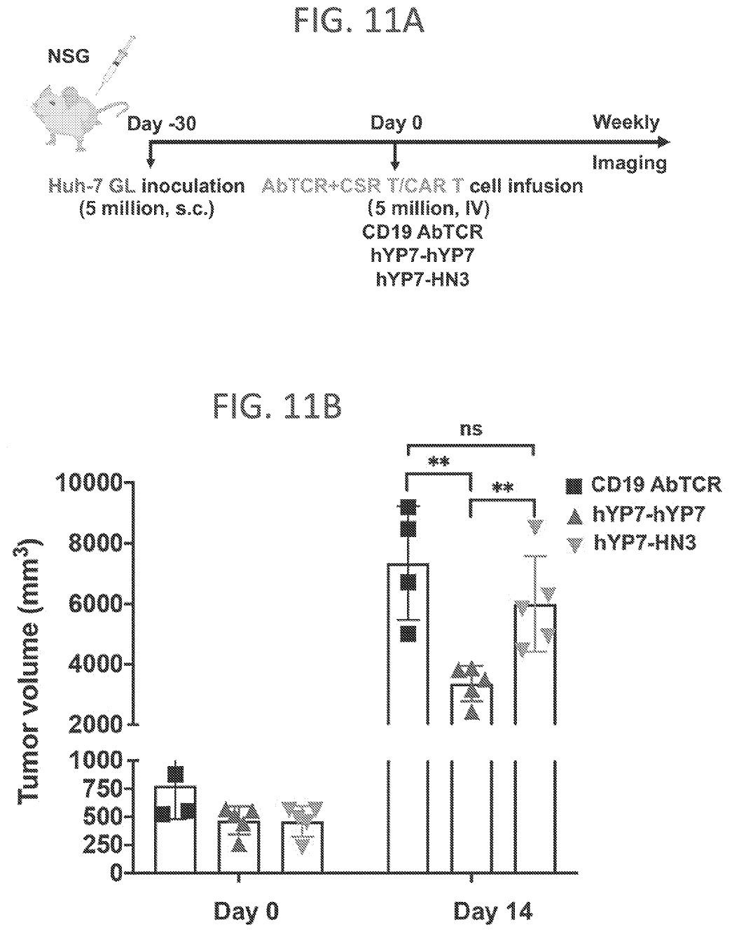

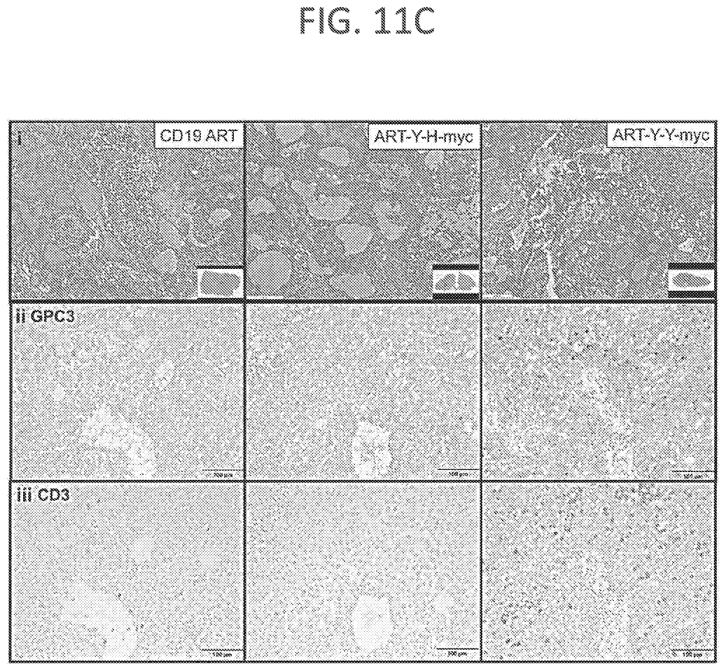

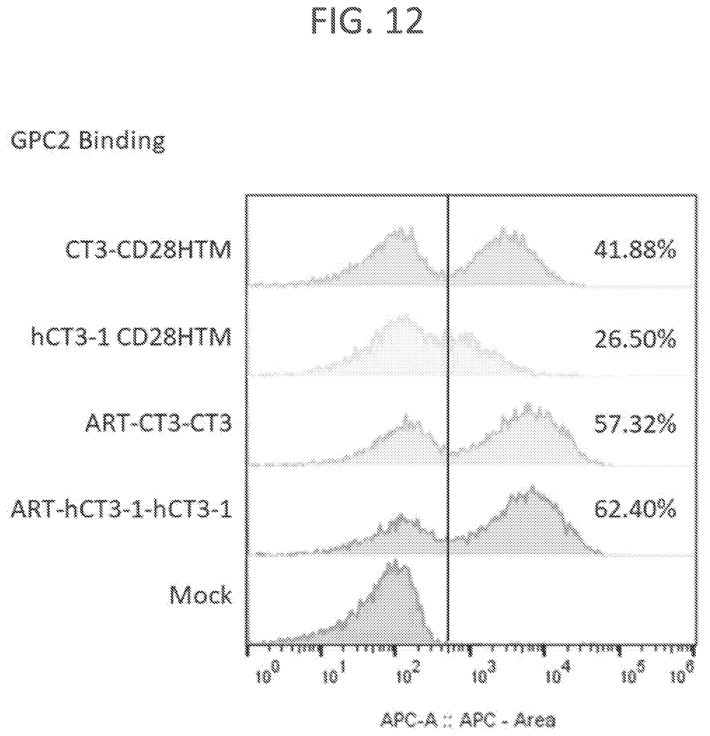

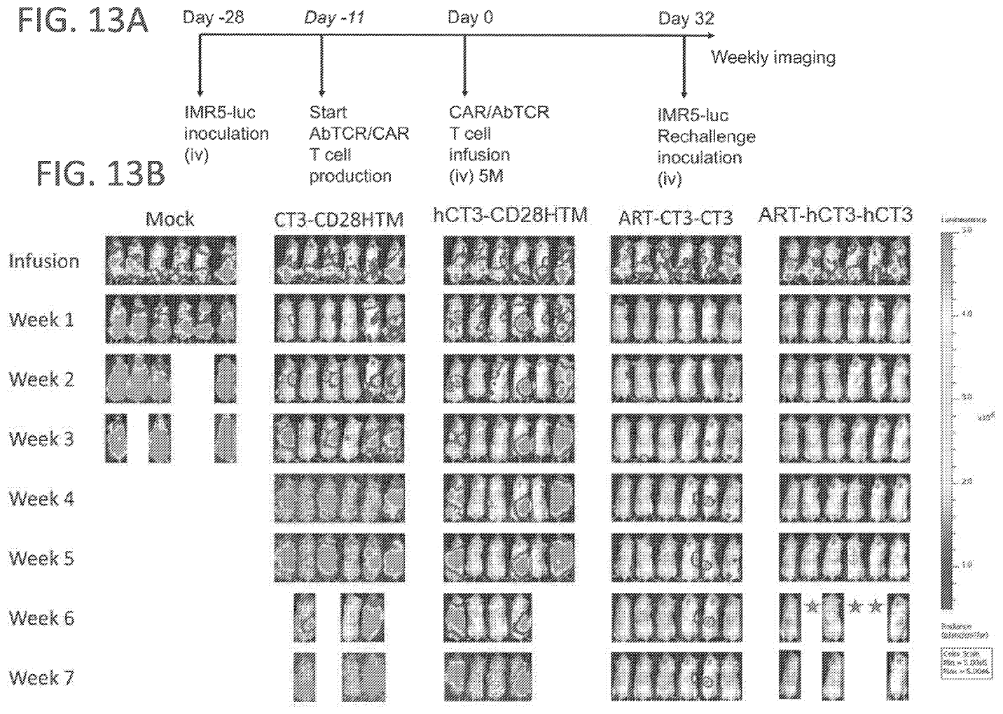

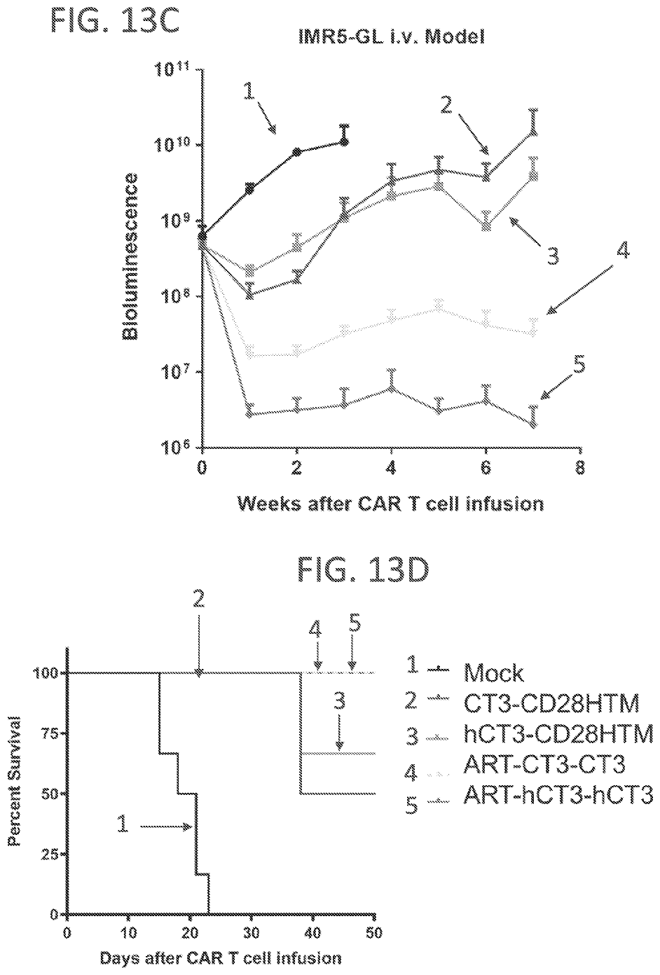

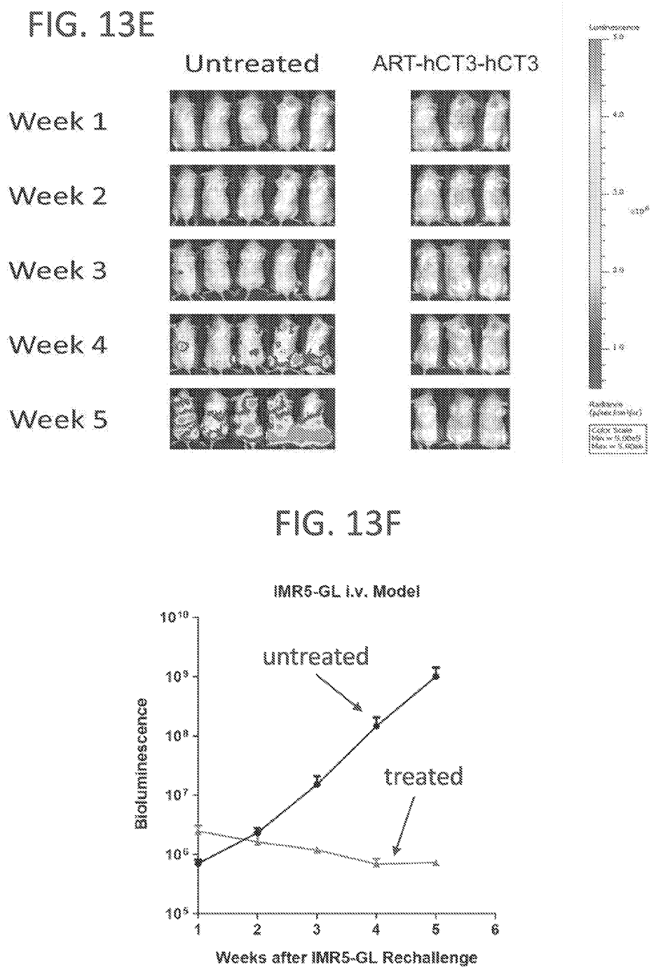

Hep3B xenograft mice were i.p. infused with 10 million CAR (hYP7), AbTCR+CSR (hYP7-hYP7), AbTCR+CSR (hYP7-HN3), AbTCR+CSR (HN3-HN3), or AbTCR+CSR (HN3-hYP7) T cells, or irrelevant CAR (CD19) T cells 12 days after tumor inoculation with 4 million Hep3B-luc cells (FIG. 7A). Representative bioluminescence images of Hep3B tumor growth up to 12 weeks post-infusion are shown in FIG. 7B. Tumor bioluminescence was measured as photons per second in CAR and AbTCR+CSR T cell treated mice and the results are shown in FIG. 7C. Mice treated with 10 million AbTCR+CSR (hYP7-hYP7) T cells and AbTCR+CSR (hYP7-HN3) T cells showed better anti-tumor activity than CAR (hYP7), AbTCR+CSR (HN3-HN3), AbTCR+CSR (HN3-hYP7), and CAR (CD19) T cell injection. FIG. 7D shows a Kaplan-Meier survival curve of mice after infusion. All GPC3-targeted AbTCR+CSRs led to greater survival compared to CAR (hYP7) T cells. Cytotoxicity of AbTCR+CSR (hYP7-hYP7) T cells was compared to cytotoxicity of AbTCR+CSR (hYP7-hYP7) T cells recovered from mouse spleen 7 weeks after injection. The AbTCR+CSR T cells were co-cultured with Hep3B cells and cytolytic activity was measured 24 hours after co-culture. CD19-specific CAR T cells were used a negative control. As shown in FIG. 7E, AbTCR+CSR (hYP7-hYP7) T cells recovered from mouse spleen 7 weeks after injection were more potent than AbTCR+CSR (hYP7-hYP7) T cells prepared in vitro.

### Example 9: AbTCR+CSR (hYP7-hYP7) T Cells Exhibit an Anti-Tumor Effect in an Orthotopic Hep3B Xenograft Model

Hep3B orthotopic xenograft mice were i.v. infused with 5 million AbTCR+CSR (hYP7-hYP7) T cells or control CAR (CD19) T cells 35 days after tumor inoculation (FIG. 8A). Imaging was performed weekly to evaluate tumor size. Representative bioluminescence images of Hep3B tumor growth up to four weeks post-infusion are shown in FIG. 8B. Tumor bioluminescence was measured as photons per second in CAR (CD19) T and AbTCR+CSR (hYP7-hYP7) T cell treated mice and the results are shown in FIG. 8C. Mice treated with 5 million AbTCR+CSR (hYP7-hYP7) T cells exhibited a significant reduction in tumor size compared to control mice treated with CAR (CD19) T cells. AbTCR+CSR (hYP7-hYP7) T cells isolated from mouse blood expressed lower levels of PD-1 than control CAR (CD19) T cells at week 3 after infusion (FIG. 8D). In mouse blood, AbTCR+CSR (hYP7-hYP7) T cells showed a higher proportion of effector memory T cells (Tₑₘ) and terminally differentiated effector memory T cells (Tₑₘᵣₐ) than CAR (CD19) T cells (FIG. 8E).

### Example 10: AbTCR+CSR (hYP7-hYP7) T Cells Potently Reduce Hep3B Subcutaneous (s.c.) Tumors

Hep3B GL cells (5 million) were subcutaneously injected into NSG mice. Hep3B xenograft mice were i.v. infused with 5 or 2.5 million AbTCR+CSR (hYP7-hYP7), AbTCR+CSR (HN3-HN3), CAR (hYP7), or control CAR (CD19) T cells 21 days after tumor inoculation (FIG. 9A). Tumor volume was measured one and two weeks after infusion of 5 million T cells (FIG. 9B). Mice (N=2) treated with 5 million AbTCR+CSR (hYP7-hYP7) T cells demonstrated tumor regression at week 2. CAR (hYP7) T cells inhibited tumor growth relative to control CD19-targeted CAR T cells, but treatment with AbTCR+CSR (HN3-HN3) T cells was ineffective. FIG. 9C shows tumor size in treated mice at the end of the study. Mice (N=5) treated with 2.5 million AbTCR+CSR (hYP7-hYP7) T cells exhibited significantly reduced tumor growth relative to mice treated with CD19 CAR T cells (FIG. 9D).

### Example 11: AbTCR+CSR (hYP7-hYP7) and AbTCR+CSR (hYP7-HN3) T Cells are More Potent than CAR (hYP7) T Cells for Treating Huh-7 s.c. Tumors

Huh-7 GL cells (low GPC3 antigen density) were intraperitoneally (i.p.) injected into NSG mice. Huh-7 xenograft mice were i.v. infused with 5 million control CAR (CD19), control AbTCR (CD19), AbTCR+CSR (hYP7-hYP7), AbTCR+CSR (hYP7-HN3) or CAR (hYP7) T cells 19 days after tumor inoculation (FIG. 10A). Treated animals were imaged weekly to assess tumor size. Huh7 tumor size measured 7, 14 and 21 days after treatment is shown in FIG. 10B. Mice treated with 5 million AbTCR+CSR (hYP7-hYP7) or AbTCR+CSR (hYP7-HN3) T cells exhibited tumor regression by week 2. Treatment with CAR (hYP7) T cells did not reduce tumor size compared to control animals treated with CD19 CAR or CD19 AbTCR T cells. FIG. 10C shows tumor size in treated mice at the end of the study. The absolute number of CAR-T or AbTCR+CSR-T cells was measured in mouse blood at week 3 of treatment (FIG. 10D). Higher T cell proliferation was elicited by treatment with GPC3 AbTCR+CSR T cells compared to treatment with GPC3 CAR T cells. AbTCR+CSR (hYP7-hYP7) T cells were more abundant than AbTCR+CSR (hYP7-HN3) T cells in treated mice. In addition, a lower level of PD-1 expression was found in ex vivo AbTCR+CSR (hYP7-hYP7) T cells compared to other treatment groups (FIG. 10E). GPC3 AbTCR+CSR T cells also showed a higher proportion of Tₑₘᵣₐ than hYP7 CAR T cells (FIG. 10F).

### Example 12: AbTCR+CSR (hYP7-hYP7 and hYP7-HN3) T Cells Slowed Down the Growth of Large Huh-7 Tumors

Huh-7 GL cells (5 million) were subcutaneously (s.c.) injected into NSG mice. Huh-7 xenograft mice were i.v. infused with 5 million AbTCR+CSR (hYP7-hYP7), AbTCR+CSR (hYP7-HN3), or control AbTCR (CD19 AbTCR) T cells 30 days after tumor inoculation (FIG. 11A). Huh7 tumor volume was measured at Day 0 and Day 14 after T cell infusion (FIG. 11B). Huh-7 tumor size on Day 0 was large (average volume of approximately 500 mm³). On Day 14, the tumor size in mice treated with irrelevant AbTCR T cells (CD19 AbTCR) increased to 7,344 mm³ per mouse on average. The hYP7-hYP7 AbTCR+CSR T cells significantly slowed down tumor growth (average tumor size of 3,358 mm³), while the hYP7-HN3 AbTCR+CSR T cells moderately slowed down tumor growth (average tumor size around 5,996 mm³). Huh-7 tumors isolated from the Huh-7 s.c. mouse model were subjected to H&E staining and IHC to detect GPC3 and CD3. After 10 days of AbTCR+CSR-T cell treatment, tumor-infiltrated hYP7-hYP7 (ART-Y-Y-myc) T cells were found in Huh-7 tumors (FIG. 11C).

It will be apparent that the precise details of the methods or compositions described may be varied or modified without departing from the spirit of the described aspects of the disclosure. We claim all such modifications and variations that fall within the scope and spirit of the claims below.

1. A nucleic acid molecule or a set of nucleic acid molecules, comprising:
(i) a first module, comprising a nucleic acid sequence encoding a first antigen-binding polypeptide, and a nucleic acid sequence encoding a co-stimulatory immune cell signaling domain;
(ii) a second module, comprising a nucleic acid sequence encoding a second antigen-binding polypeptide, and a nucleic acid sequence encoding a first T cell receptor (TCR) chain or a transmembrane domain-containing fragment thereof; and
(iii) a third module, comprising a nucleic acid sequence encoding a third antigen-binding polypeptide, and a nucleic acid sequence encoding a second TCR chain or a transmembrane domain-containing fragment thereof,
wherein the first, second and third antigen-binding polypeptides specifically bind glypican-2 (GPC2), or the first, second and third antigen-binding polypeptides specifically bind glypican-3 (GPC3).

2. The nucleic acid molecule or set of nucleic acid molecules of claim 1, wherein the first, second and third antigen-binding polypeptides specifically bind GPC2, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 10 and the VL domain comprises the CDR1, CDR2 and CDR3 sequences of SEQ ID NO: 11;
the second antigen-binding polypeptide comprises a VH domain and a heavy chain constant region, wherein the VH domain comprises the CDR1, CDR2 and CDR3 sequences of SEQ ID NO: 10; and
the third antigen-binding polypeptide comprises a VL domain and a light chain constant region, wherein the VL domain comprises the CDR1, CDR2 and CDR3 sequences of SEQ ID NO: 11.

3. The nucleic acid molecule or set of nucleic acid molecules of claim 2, wherein:
the amino acid sequence of the VH domain comprises or consists of SEQ ID NO: 10 and the amino acid sequence of the VL domain comprises or consists of SEQ ID NO: 11; or
the amino acid sequence of the VH domain comprises or consists of SEQ ID NO: 7 and the amino acid sequence of the VL domain comprises or consists of SEQ ID NO: 8.

4. (canceled)

5. The nucleic acid molecule or set of nucleic acid molecules of claim 1, wherein the first, second and third antigen-binding polypeptides specifically bind GPC3, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 13 and the VL domain comprises the CDR1, CDR2 and CDR3 sequences of SEQ ID NO: 14;
the second antigen-binding polypeptide comprises a VH domain and a heavy chain constant region, wherein the VH domain comprises the CDR1, CDR2 and CDR3 sequences of SEQ ID NO: 13; and
the third antigen-binding polypeptide comprises a VL domain and a light chain constant region, wherein the VL domain comprises the CDR1, CDR2 and CDR3 sequences of SEQ ID NO: 14.

6. The nucleic acid molecule or set of nucleic acid molecules of claim 5, wherein:
the amino acid sequence of the VH domain of the first antigen-binding polypeptide comprises or consists of SEQ ID NO: 13 and the amino acid sequence of the VL domain of the first antigen-binding polypeptide comprises or consists of SEQ ID NO: 14;
the amino acid sequence of the VH domain of the second antigen-binding polypeptide comprises or consists of SEQ ID NO: 13; and
the amino acid sequence of the VL domain of the third antigen-binding polypeptide comprises or consists of SEQ ID NO: 14.

7. (canceled)

8. The nucleic acid molecule or set of nucleic acid molecules of claim 1, wherein the first, second and third antigen-binding polypeptides specifically bind GPC3, wherein:
the first antigen-binding polypeptide comprises a single-domain antibody comprising the CDR1, CDR2 and CDR3 sequences of SEQ ID NO: 16;
the second antigen-binding polypeptide comprises a VH domain and a heavy chain constant region, wherein the VH domain comprises the CDR1, CDR2 and CDR3 sequences of SEQ ID NO: 13; and
the third antigen-binding polypeptide comprises a VL domain and a light chain constant region, wherein the VL domain comprises the CDR1, CDR2 and CDR3 sequences of SEQ ID NO: 14.

9. The nucleic acid molecule or set of nucleic acid molecules of claim 8, wherein:
the amino acid sequence of the single-domain antibody of the first antigen-binding polypeptide comprises or consists of SEQ ID NO: 16;
the amino acid sequence of the VH domain of the second antigen-binding polypeptide comprises or consists of SEQ ID NO: 13; and
the amino acid sequence of the VL domain of the third antigen-binding polypeptide comprises or consists of SEQ ID NO: 14.

10. The nucleic acid molecule or set of nucleic acid molecules of claim 1, wherein the first, second and third antigen-binding polypeptides specifically bind GPC3, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 13 and the VL domain comprises the CDR1, CDR2 and CDR3 sequences of SEQ ID NO: 14;
the second antigen-binding polypeptide comprises a single-domain antibody and a heavy chain constant region, wherein the single-domain antibody comprises the CDR1, CDR2 and CDR3 sequences of SEQ ID NO: 16; and
the third antigen-binding polypeptide comprises a single-domain antibody and a light chain constant region, wherein the single-domain antibody comprises the CDR1, CDR2 and CDR3 sequences of SEQ ID NO: 16.

11. The nucleic acid molecule or set of nucleic acid molecules of claim 10, wherein:
the amino acid sequence of the VH domain of the first antigen-binding polypeptide comprises or consists of SEQ ID NO: 13; and the amino acid sequence of the VL domain of the first antigen-binding polypeptide comprises or consists of SEQ ID NO: 14;
the amino acid sequence of the single-domain antibody of the second antigen-binding polypeptide comprises or consists of SEQ ID NO: 16; and
the amino acid sequence of the single-domain antibody of the third antigen-binding polypeptide comprises or consists of SEQ ID NO: 16.

12. (canceled)

13. The nucleic acid molecule or set of nucleic acid molecules of claim 1, wherein the first, second and third antigen-binding polypeptides specifically bind GPC3, wherein:
the first antigen-binding polypeptide comprises a single-domain antibody comprising the CDR1, CDR2 and CDR3 sequences of SEQ ID NO: 16;
the second antigen-binding polypeptide comprises a single-domain antibody and a heavy chain constant region, wherein the single-domain antibody comprises the CDR1, CDR2 and CDR3 sequences of SEQ ID NO: 16; and
the third antigen-binding polypeptide comprises a single-domain antibody and a light chain constant region, wherein the single-domain antibody comprises the CDR1, CDR2 and CDR3 sequences of SEQ ID NO: 16.

14. The nucleic acid molecule or set of nucleic acid molecules of claim 13, wherein:
the amino acid sequence of the single-domain antibody of the first antigen-binding polypeptide comprises or consists of SEQ ID NO: 16;
the amino acid sequence of the single-domain antibody of the second antigen-binding polypeptide comprises or consists of SEQ ID NO: 16; and
the amino acid sequence of the single-domain antibody of the third antigen-binding polypeptide comprises or consists of SEQ ID NO: 16.

15. The nucleic acid molecule or set of nucleic acid molecules of claim 2, wherein:
the amino acid sequence of the heavy chain constant region comprises or consists of SEQ ID NO: 17; and/or
the amino acid sequence of the light chain constant region comprises or consists of SEQ ID NO: 18.

16-17. (canceled)

18. The nucleic acid molecule or set of nucleic acid molecules of claim 1, wherein the co-stimulatory immune cell signaling domain comprises all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

19. The nucleic acid molecule or set of nucleic acid molecules of claim 1, wherein:
(1) the first TCR chain or the transmembrane domain-containing fragment thereof is derived from a TCR γ chain, and the second TCR chain or the transmembrane domain-containing fragment thereof is derived from a TCR δ chain; or
(2) the first TCR chain or the transmembrane domain-containing fragment thereof is derived from a TCR δ chain, and the second TCR chain or the transmembrane domain-containing fragment thereof is derived from a TCR γ chain.

20. The nucleic acid molecule or set of nucleic acid molecules of claim 1, wherein:
(1) the first TCR chain or the transmembrane domain-containing fragment thereof is derived from a TCR α chain, and the second TCR chain or the transmembrane domain-containing fragment thereof is derived from a TCR β chain; or
(2) the first TCR chain or the transmembrane domain-containing fragment thereof is derived from a TCR β chain, and the second TCR chain or the transmembrane domain-containing fragment thereof is derived from a TCR α chain.

21-22. (canceled)

23. The nucleic acid molecule of claim 1, wherein the first, second and third modules are each separated by a nucleic acid sequence encoding a self-cleaving peptide.

24-25. (canceled)

26. The nucleic acid molecule of claim 1, encoding the amino acid sequence of SEQ ID NO: 2, SEQ ID NO: 1, SEQ ID NO: 3, SEQ ID NO: 4, SEQ ID NO: 5 or SEQ ID NO: 6.

27. (canceled)

28. A vector or set of vectors, comprising the nucleic acid molecule or set of nucleic acid molecules of claim 1.

29. An isolated cell comprising the nucleic acid molecule or set of nucleic acid molecules of claim 1.

30. The isolated cell of claim 29, wherein the cell is an immune cell or an induced pluripotent stem cell (iPSC).

31. (canceled)

32. A composition comprising the isolated cell of claim 30, and a pharmaceutically acceptable carrier.

33. An isolated immune cell or iPSC expressing:
(i) a first chimeric polypeptide chain, comprising a first antigen-binding polypeptide and a co-stimulatory immune cell signaling domain;
(ii) a second chimeric polypeptide chain, comprising a second antigen-binding polypeptide and a first T cell receptor (TCR) chain or a transmembrane domain-containing fragment thereof; and
(iii) a third chimeric polypeptide chain, comprising a third antigen-binding polypeptide and a second TCR chain or a transmembrane domain-containing fragment thereof,
wherein the first, second and third antigen-binding polypeptides specifically bind glypican-2 (GPC2), or the first, second and third antigen-binding polypeptides specifically bind glypican-3 (GPC3).

34. The isolated immune cell or iPSC of claim 33, wherein the first, second and third antigen-binding polypeptides specifically bind GPC2, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 10 and the VL domain comprises the CDR1, CDR2 and CDR3 sequences of SEQ ID NO: 11;
the second antigen-binding polypeptide comprises a VH domain and a heavy chain constant region, wherein the VH domain comprises the CDR1, CDR2 and CDR3 sequences of SEQ ID NO: 10; and
the third antigen-binding polypeptide comprises a VL domain and a light chain constant region, wherein the VL domain comprises the CDR1, CDR2 and CDR3 sequences of SEQ ID NO: 11.

35. The isolated immune cell of iPSC of claim 34, wherein:
the amino acid sequence of the VH domain comprises or consists of SEQ ID NO: 10 and the amino acid sequence of the VL domain comprises or consists of SEQ ID NO: 11; or
the amino acid sequence of the VH domain comprises or consists of SEQ ID NO: 7 and the amino acid sequence of the VL domain comprises or consists of SEQ ID NO: 8

36. (canceled)

37. The isolated immune cell or iPSC of claim 33, wherein the first, second and third antigen-binding polypeptides specifically bind GPC3, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 13 and the VL domain comprises the CDR1, CDR2 and CDR3 sequences of SEQ ID NO: 14;
the second antigen-binding polypeptide comprises a VH domain and a heavy chain constant region, wherein the VH domain comprises the CDR1, CDR2 and CDR3 sequences of SEQ ID NO: 13; and
the third antigen-binding polypeptide comprises a VL domain and a light chain constant region, wherein the VL domain comprises the CDR1, CDR2 and CDR3 sequences of SEQ ID NO: 14.

38. The isolated immune cell or iPSC of claim 37, wherein:
the amino acid sequence of the VH domain of the first antigen-binding polypeptide comprises or consists of SEQ ID NO: 13 and the amino acid sequence of the VL domain of the first-antigen-binding polypeptide comprises or consists of SEQ ID NO: 14;
the amino acid sequence of the VH domain of the second antigen-binding polypeptide comprises or consists of SEQ ID NO: 13; and
the amino acid sequence of the VL domain of the third antigen-binding polypeptide comprises or consists of SEQ ID NO: 14.

39. (canceled)

40. The isolated immune cell or iPSC of claim 33, wherein the first, second and third antigen-binding polypeptides specifically bind GPC3, wherein:
the first antigen-binding polypeptide comprises a single-domain antibody comprising the CDR1, CDR2 and CDR3 sequences of SEQ ID NO: 16;
the second antigen-binding polypeptide comprises a VH domain and a heavy chain constant region, wherein the VH domain comprises the CDR1, CDR2 and CDR3 sequences of SEQ ID NO: 13; and
the third antigen-binding polypeptide comprises a VL domain and a light chain constant region, wherein the VL domain comprises the CDR1, CDR2 and CDR3 sequences of SEQ ID NO: 14.

41. The isolated immune cell or iPSC of claim 40, wherein:
the amino acid sequence of the single-domain antibody of the first antigen-binding polypeptide comprises or consists of SEQ ID NO: 16;
the amino acid sequence of the VH domain of the second antigen-binding polypeptide comprises or consists of SEQ ID NO: 13; and
the amino acid sequence of the VL domain of the third antigen-binding polypeptide comprises or consists of SEQ ID NO: 14.

42. The isolated immune cell or iPSC of claim 33, wherein the first, second and third antigen-binding polypeptides specifically bind GPC3, wherein:
the first antigen-binding polypeptide comprises a single-chain variable fragment (scFv) comprising a variable heavy (VH) domain and a variable light (VL) domain, and the VH domain comprises the CDR1, CDR2 and CDR3 sequences of SEQ ID NO: 13 and the VL domain comprises the CDR1, CDR2 and CDR3 sequences of SEQ ID NO: 14;
the second antigen-binding polypeptide comprises a single-domain antibody and a heavy chain constant region, wherein the single-domain antibody comprises the CDR1, CDR2 and CDR3 sequences of SEQ ID NO: 16; and
the third antigen-binding polypeptide comprises a single-domain antibody and a light chain constant region, wherein the single-domain antibody comprises the CDR1, CDR2 and CDR3 sequences of SEQ ID NO: 16.

43. The isolated immune cell or iPSC of claim 42, wherein:
the amino acid sequence of the VH domain of the first antigen-binding polypeptide comprises or consists of SEQ ID NO: 13; and the amino acid sequence of the VL domain of the first antigen-binding polypeptide comprises or consists of SEQ ID NO: 14;
the amino acid sequence of the single-domain antibody of the second antigen-binding polypeptide comprises or consists of SEQ ID NO: 16; and
the amino acid sequence of the single-domain antibody of the third antigen-binding polypeptide comprises or consists of SEQ ID NO: 16.

44. (canceled)

45. The isolated immune cell or iPSC of claim 33, wherein the first, second and third antigen-binding polypeptides specifically bind GPC3, wherein:
the first antigen-binding polypeptide comprises a single-domain antibody comprising the CDR1, CDR2 and CDR3 sequences of SEQ ID NO: 16;
the second antigen-binding polypeptide comprises a single-domain antibody and a heavy chain constant region, wherein the single-domain antibody comprises the CDR1, CDR2 and CDR3 sequences of SEQ ID NO: 16; and
the third antigen-binding polypeptide comprises a single-domain antibody and a light chain constant region, wherein the single-domain antibody comprises the CDR1, CDR2 and CDR3 sequences of SEQ ID NO: 16.

46. The isolated immune cell or iPSC of claim 45, wherein:
the amino acid sequence of the single-domain antibody of the first antigen-binding polypeptide comprises or consists of SEQ ID NO: 16;
the amino acid sequence of the single-domain antibody of the second antigen-binding polypeptide comprises or consists of SEQ ID NO: 16; and
the amino acid sequence of the single-domain antibody of the third antigen-binding polypeptide comprises or consists of SEQ ID NO: 16.

47. The isolated immune cell or iPSC of claim 34, wherein:
the amino acid sequence of the heavy chain constant region comprises or consists of SEQ ID NO: 17; and/or
the amino acid sequence of the light chain constant region comprises or consists of SEQ ID NO: 18.

48-49. (canceled)

50. The isolated immune cell or iPSC of claim 33, wherein the co-stimulatory immune cell signaling domain comprises all or a portion of the intracellular signaling domain of CD30, CD27, CD28, 4-1BB (CD137), OX40, CD40, ICOS, lymphocyte function-associated antigen-1 (LFA-1), CD2, CD7, LIGHT, NKG2C, B7-H3, or a ligand that specifically binds with CD83.

51. The isolated immune cell or iPSC of claim 33, wherein:
(1) the first TCR chain or the transmembrane domain-containing fragment thereof is derived from a TCR γ chain, and the second TCR chain or the transmembrane domain-containing fragment thereof is derived from a TCR δ chain; or
(2) the first TCR chain or the transmembrane domain-containing fragment thereof is derived from a TCR δ chain, and the second TCR chain or the transmembrane domain-containing fragment thereof is derived from a TCR γ chain.

52. The isolated immune cell or iPSC of claim 33, wherein:
(1) the first TCR chain or the transmembrane domain-containing fragment thereof is derived from a TCR α chain, and the second TCR chain or the transmembrane domain-containing fragment thereof is derived from a TCR β chain; or
(2) the first TCR chain or the transmembrane domain-containing fragment thereof is derived from a TCR β chain, and the second TCR chain or the transmembrane domain-containing fragment thereof is derived from a TCR α chain.

53. The isolated immune cell or iPSC of claim 33, wherein:
the amino acid sequence of the first chimeric polypeptide chain comprises residues 21-556 of SEQ ID NO: 2, the amino acid sequence of the second chimeric polypeptide chain comprises residues 604-897 of SEQ ID NO: 2, and the amino acid sequence of the third chimeric polypeptide chain comprises residues 944-1223 of SEQ ID NO: 2;
the amino acid sequence of the first chimeric polypeptide chain comprises residues 21-556 of SEQ ID NO: 1, the amino acid sequence of the second chimeric polypeptide chain comprises residues 604-897 of SEQ ID NO: 1, and the amino acid sequence of the third chimeric polypeptide chain comprises residues 944-1223 of SEQ ID NO: 1;
the amino acid sequence of the first chimeric polypeptide chain comprises residues 21-557 of SEQ ID NO: 3, the amino acid sequence of the second chimeric polypeptide chain comprises residues 605-892 of SEQ ID NO: 3, and the amino acid sequence of the third chimeric polypeptide chain comprises residues 939-1225 of SEQ ID NO: 3;
the amino acid sequence of the first chimeric polypeptide chain comprises residues 21-421 of SEQ ID NO: 4, the amino acid sequence of the second chimeric polypeptide chain comprises residues 469-756 of SEQ ID NO: 4, and the amino acid sequence of the third chimeric polypeptide chain comprises residues 803-1089 of SEQ ID NO: 4;
the amino acid sequence of the first chimeric polypeptide chain comprises residues 21-557 of SEQ ID NO: 5, the amino acid sequence of the second chimeric polypeptide chain comprises residues 605-891 of SEQ ID NO: 5, and the amino acid sequence of the third chimeric polypeptide chain comprises residues 938-1227 of SEQ ID NO: 5; or
the amino acid sequence of the first chimeric polypeptide chain comprises residues 21-421 of SEQ ID NO: 6, the amino acid sequence of the second chimeric polypeptide chain comprises residues 469-755 of SEQ ID NO: 6, and the amino acid sequence of the third chimeric polypeptide chain comprises residues 802-1091 of SEQ ID NO: 6.

54. (canceled)

55. A composition comprising the isolated immune cell or iPSC of claim 33 and a pharmaceutically acceptable carrier.

56. A method of treating a GPC2-positive or GPC3-positive cancer in a subject, or inhibiting tumor growth and/or metastasis of a GPC2-positive or GPC3-positive cancer in a subject, comprising administering to the subject a therapeutically effective amount of the isolated immune cell or iPSC of claim 30, thereby treating the GPC2-positive or GPC3-positive cancer.

57. (canceled)

58. The method of claim 56, wherein:
the GPC2-positive cancer is neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma, small cell lung cancer, or osteosarcoma; or
the GPC3-positive cancer is a hepatocellular carcinoma (HCC), melanoma, ovarian clear-cell carcinoma, yolk sac tumor (YST), hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma, salivary gland cancer, glioblastoma, choriocarcinoma, rhabdosarcoma, renal clear-cell carcinoma, or embryonal tumor.

59. (canceled)


SEQUENCE LISTING (53 sequences)

SEQ ID NO: 1 - protein, 1223 aa, synthetic construct
  REGION 1..1223: note = Synthetic polypeptide
METDTLLLWVLLLWVPGSTGENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKRSRGGGGSGGGGSGGGGSLEMAEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 2 - protein, 1223 aa, synthetic construct
  REGION 1..1223: note = Synthetic polypeptide
METDTLLLWVLLLWVPGSTGDVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIKRSRGGGGSGGGGSGGGGSLEMAQVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGQVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGDVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 3 - protein, 1225 aa, synthetic construct
  REGION 1..1225: note = Synthetic polypeptide
METDTLLLWVLLLWVPGSTGDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKRSRGGGGSGGGGSGGGGSLEMAEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 4 - protein, 1089 aa, synthetic construct
  REGION 1..1089: note = Synthetic polypeptide
METDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 5 - protein, 1227 aa, synthetic construct
  REGION 1..1227: note = Synthetic polypeptide
METDTLLLWVLLLWVPGSTGDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKRSRGGGGSGGGGSGGGGSLEMAEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 6 - protein, 1091 aa, synthetic construct
  REGION 1..1091: note = Synthetic polypeptide
METDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSEQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKRAKRSGSGATNFSLLKQAGDVEENPGPMETDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFLRAKRSGSGEGRGSLLTCGDVEENPGPMETDTLLLWVLLLWVPGSTGQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPIKTDVITMDPKDNCSKDANDTLLLQLTNTSAYYMYLLLLLKSVVYFAIITCCLLRRTAFCCNGEKS

SEQ ID NO: 7 - protein, 123 aa, synthetic construct
  REGION 1..123: note = Synthetic polypeptide
EVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSS

SEQ ID NO: 8 - protein, 106 aa, synthetic construct
  REGION 1..106: note = Synthetic polypeptide
ENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELK

SEQ ID NO: 9 - protein, 251 aa, synthetic construct
  REGION 1..251: note = Synthetic polypeptide
ENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKRSRGGGGSGGGGSGGGGSLEMAEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSS

SEQ ID NO: 10 - protein, 123 aa, synthetic construct
  REGION 1..123: note = Synthetic polypeptide
QVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSS

SEQ ID NO: 11 - protein, 106 aa, synthetic construct
  REGION 1..106: note = Synthetic polypeptide
DVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIK

SEQ ID NO: 12 - protein, 251 aa, synthetic construct
  REGION 1..251: note = Synthetic polypeptide
DVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIKRSRGGGGSGGGGSGGGGSLEMAQVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSS

SEQ ID NO: 13 - protein, 117 aa, synthetic construct
  REGION 1..117: note = Synthetic polypeptide
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 14 - protein, 113 aa, synthetic construct
  REGION 1..113: note = Synthetic polypeptide
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 15 - protein, 252 aa, synthetic construct
  REGION 1..252: note = Synthetic polypeptide
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKRSRGGGGSGGGGSGGGGSLEMAEVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 16 - protein, 116 aa, synthetic construct
  REGION 1..116: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSS

SEQ ID NO: 17 - protein, 107 aa, synthetic construct
  REGION 1..107: note = Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKT

SEQ ID NO: 18 - protein, 107 aa, synthetic construct [sequence omitted]

SEQ ID NO: 19 - protein, 64 aa, synthetic construct
  REGION 1..64: note = Synthetic polypeptide
DSTDHVKPKETENTKQPSKSCHKPKAIVHTEKVNMMSLTVLGLRMLFAKTVAVNFLLTAKLFFL

SEQ ID NO: 20 - protein, 67 aa, synthetic construct
  REGION 1..67: note = Synthetic polypeptide
PIKTDVITMDPKDNCSKDANDTLLLQLTNTSAYYMYLLLLLKSVVYFAIITCCLLRRTAFCCNGEKS

SEQ ID NO: 21 - protein, 275 aa, synthetic construct
  REGION 1..275: note = Synthetic polypeptide
AAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 22 - protein, 20 aa, synthetic construct
  REGION 1..20: note = Synthetic polypeptide
METDTLLLWVLLLWVPGSTG

SEQ ID NO: 23 - protein, 10 aa, synthetic construct
  REGION 1..10: note = Synthetic polypeptide
EQKLISEEDL

SEQ ID NO: 24 - protein, 22 aa, synthetic construct
  REGION 1..22: note = Synthetic polypeptide
GSGATNFSLLKQAGDVEENPGP

SEQ ID NO: 25 - protein, 21 aa, synthetic construct
  REGION 1..21: note = Synthetic polypeptide
GSGEGRGSLLTCGDVEENPGP

SEQ ID NO: 26 - DNA, 369 bp, synthetic construct
  misc_feature 1..369: note = Synthetic nucleic acid
  source 1..369: mol_type = other DNA
gaggtccagctgcaacagtctggacctgaactggtgaagcctggggcttcagtaaagatgtcctgcaaggcttctagattcacattcactgactacaacatacactgggtgaagcagagccctggaaagacccttgaatggattggatatattaaccctaacaatggtgatattttctacaaacagaagttcaatggcaaggccacattgactataaacaagtcctccaacacagcctacatggagctccgcagcctgacatcggaggattctgcagtctattactgtgtaagatcctctaatattcgttatactttcgacaggttcttcgatgtctggggcacagggaccacggtcaccgtctcctca

SEQ ID NO: 27 - DNA, 318 bp, synthetic construct
  misc_feature 1..318: note = Synthetic nucleic acid
  source 1..318: mol_type = other DNA
gaaaatgtgctcacccagtctccagcaatcatgtctgcatctctaggggagaaggtcaccatgagctgcagggccagctcaagtgtaaattacatttactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagttttctagttccccatccacgttcggtactgggaccaagctggagctgaaa

SEQ ID NO: 28 - DNA, 369 bp, synthetic construct
  misc_feature 1..369: note = Synthetic nucleic acid
  source 1..369: mol_type = other DNA
caagtacagcttgtacaatccggagcggaagtaaagaagcccggggcctccgtgaaagtcagctgcaaagcgtctagattcaccttcactgactataacatccactgggtgcggcaagcgcctggtcagggcctcgaatggattggctatataaacccgaacaacggggacattttctataagcagaaattcaatggcagagtgacgctcaccgcggacaaaagtaccagtactgcttatatggaactgtctagtcttacgagtgaggataccgctgtgtattattgcgtgaggtcctccaatatacgctatacgtttgatagattctttgatgtatggggacagggaaccctcgtcacggtcagctca

SEQ ID NO: 29 - DNA, 318 bp, synthetic construct
  misc_feature 1..318: note = Synthetic nucleic acid
  source 1..318: mol_type = other DNA
gacgtagtaatgactcaaagccccctctccttgccagtgacgccaggtgagccggcatctatttcttgcagggctagttcttcagtgaactacatctattggtatttgcagaagccaggacaaagtccgcagctttggatatattacacgagcaaccttgcgccaggtgttccagatcgcttctctggcagtggatcagggacggacttcacactgaagatctctcgcgttgaagctgaagacgtcggcgtgtactattgccaacaattcagtagttctccgagcacttttggccagggaaccaagcttgagattaaa

SEQ ID NO: 30 - DNA, 351 bp, synthetic construct
  misc_feature 1..351: note = Synthetic nucleic acid
  source 1..351: mol_type = other DNA
gaggtgcagcttgttgagtctggtggaggattggtgcagcctggagggtcattgagactctcatgtgcagcctctggattcaccttcaataagaatgccatgaattgggtccgccaggctccaggaaagggtttggaatgggttggccgcataagaaataaaactaataattatgcaacatattatgccgattcagtgaaagccaggtttaccatctccagagatgattcaaagaactcactctatctgcaaatgaacagcttgaaaaccgaggacacagccgtgtactattgtgtggctggtaactcgtttgcttactggggccaagggactctggtcactgtctctgca

SEQ ID NO: 31 - DNA, 339 bp, synthetic construct
  misc_feature 1..339: note = Synthetic nucleic acid
  source 1..339: mol_type = other DNA
gacattgtgatgacccagtctccagactccctagctgtgtcactgggagagagggccactatcaactgcaagtccagtcagagccttttatatagcagcaatcaaaagaactacttggcctggtaccaacagaaaccagggcagcctcctaaactgctgatttactgggcatccagtagggaatctggggtccctgatcgcttcagtggcagtggatctgggacagatttcactctcaccatcagcagtctgcaggctgaagacgtggcagtttattactgtcagcaatattataactatccgctcacgttcggtcaggggaccaagttggagatcaaa

SEQ ID NO: 32 - DNA, 351 bp, synthetic construct
  misc_feature 1..351: note = Synthetic nucleic acid
  source 1..351: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaagt

SEQ ID NO: 33 - DNA, 321 bp, synthetic construct
  misc_feature 1..321: note = Synthetic nucleic acid
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 34 - DNA, 123 bp, synthetic construct
  misc_feature 1..123: note = Synthetic nucleic acid
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 35 - DNA, 282 bp, synthetic construct
  misc_feature 1..282: note = Synthetic nucleic acid
  source 1..282: mol_type = other DNA
ccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 36 - DNA, 126 bp, synthetic construct
  misc_feature 1..126: note = Synthetic nucleic acid
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 37 - DNA, 810 bp, synthetic construct
  misc_feature 1..810: note = Synthetic nucleic acid
  source 1..810: mol_type = other DNA
gcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 38 - DNA, 564 bp, synthetic construct
  misc_feature 1..564: note = Synthetic nucleic acid
  source 1..564: mol_type = other DNA
caccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaag

SEQ ID NO: 39 - DNA, 180 bp, synthetic construct
  misc_feature 1..180: note = Synthetic nucleic acid
  source 1..180: mol_type = other DNA
attccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 40 - DNA, 153 bp, synthetic construct
  misc_feature 1..153: note = Synthetic nucleic acid
  source 1..153: mol_type = other DNA
ggtagagcagactgtggctttacctcggtgtcctaccaacaaggggtcctgtctgccaccatcctctatgagatcctactagggaaggccaccctgtatgctgtgctggtcagcgcccttgtgttgatggccatggtcaagagaaaggatttc

SEQ ID NO: 41 - DNA, 192 bp, synthetic construct
  misc_feature 1..192: note = Synthetic nucleic acid
  source 1..192: mol_type = other DNA
gattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 42 - DNA, 72 bp, synthetic construct
  misc_feature 1..72: note = Synthetic nucleic acid
  source 1..72: mol_type = other DNA
gtgcttgggctacgaatgctgtttgcaaagactgttgccgtcaattttctcttgactgccaagttatttttc

SEQ ID NO: 43 - DNA, 201 bp, synthetic construct
  misc_feature 1..201: note = Synthetic nucleic acid
  source 1..201: mol_type = other DNA
cccataaaaaccgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatca

SEQ ID NO: 44 - DNA, 71 bp, synthetic construct
  misc_feature 1..71: note = Synthetic nucleic acid
  source 1..71: mol_type = other DNA
ttattacatgtacctcctcctgctcctcaagagtgtggtctattttgccatcatcacctgctgtctgcttg

SEQ ID NO: 45 - DNA, 321 bp, synthetic construct
  misc_feature 1..321: note = Synthetic nucleic acid
  source 1..321: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaaaagagttgaacccaaaagttgtgaagttaaaact

SEQ ID NO: 46 - DNA, 321 bp, synthetic construct
  misc_feature 1..321: note = Synthetic nucleic acid
  source 1..321: mol_type = other DNA
aggactgttgctgctccatctgtttttatttttccaccatctgatgaacaacttaaatctggaactgcttctgttgtttgtcttcttaataatttttatccaagagaagctaaagttcaatggaaagttgataatgctcttcaatctggaaattctcaagaatctgttactgaacaagattctaaagattctacttattctctttcttctactcttactctttctaaagctgattatgaaaaacataaagtttatgcttgtgaagttactcatcaaggactttcttctccagttactaaatcttttaatagaggagaatgt

SEQ ID NO: 47 - protein, 61 aa, synthetic construct
  REGION 1..61: note = Synthetic protein
IIPEDTFFPSPESSCDVKLVEKSFETDTNLNFQNLSVIGFRILLLKVAGFNLLMTLRLWSS

SEQ ID NO: 48 - protein, 51 aa, synthetic construct
  REGION 1..51: note = Synthetic protein
GRADCGFTSVSYQQGVLSATILYEILLGKATLYAVLVSALVLMAMVKRKDF

SEQ ID NO: 49 - protein, 189 aa, synthetic construct
  REGION 1..189: note = Synthetic protein
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 50 - protein, 107 aa, synthetic construct
  REGION 1..107: note = Synthetic protein
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 51 - protein, 41 aa, synthetic construct
  REGION 1..41: note = Synthetic protein
RSKRSRLLHSDYMNMTPRRPGPTRKHYQPYAPPRDFAAYRS

SEQ ID NO: 52 - protein, 94 aa, synthetic construct
  REGION 1..94: note = Synthetic protein
PADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 53 - protein, 42 aa, synthetic construct
  REGION 1..42: note = Synthetic protein
KRGRKKLLYIFKQPFMRPVQTTQEEDGCSCRFPEEEEGGCEL